(19) United States
(12) Patent Application Publication
(10) Pub. No.: US 2002/0132845 A1
(43) Pub. Date: Sep. 19, 2002

(54) Compositions and methods for the prevention and treatment of tissue ischemia

(76) Inventors: Guy Michael Miller, San Jose, CA (US); Lesley A. Brown, San Jose, CA (US); Ughetta Del Balzo, Morgan Hill, CA (US); Stephen Flaim, San Diego, CA (US); Sekhar Boddupalli, San Jose, CA (US); Bing Wang, Cupertino, CA (US)

Correspondence Address: Gladys H. Monroy, Morrison & Foerster LLP, 755 Page Mill Road, Palo Alto, CA, 94304-1018, US

(21) Appl. No.: 10/017,717

(22) Filed: Dec. 14, 2001

Related U.S. Application Data
(60) Provisional application No. 60/256,269, filed on Dec. 15, 2000.
(60) Provisional application No. 60/296,581, filed on Jun. 6, 2001.
(60) Provisional application No. 60/296,580, filed on Jun. 6, 2001.
(60) Provisional application No. 60/343,575, filed on Oct. 19, 2001.

(51) Int. Cl. A61K 31/355

(52) U.S. Cl. 514/458

(57) ABSTRACT

The present invention provides compositions and methods for the treatment of tissue ischemia, and in particular, cerebral ischemia. In particular, the present invention provides gamma-, beta-, or delta-tocopherol enriched tocopherol compositions and gamma-, beta-, or delta-tocopherol metabolite enriched compositions and/or flavonoid enriched and/or a flavonoid derivative enriched compositions and methods for their use in preventing or treating a tissue ischemic condition or a cerebral ischemic condition. The present invention also provides pharmaceutical compositions comprising gamma-, beta-, or delta-tocopherol enriched tocopherol composition, a gamma-, beta-, or delta-tocopherol metabolite enriched compositions or flavonoid enriched compositions or flavonoid derivative enriched compositions.

### CROSS-REFERENCE TO RELATED APPLICATIONS

This application claims the benefit of U.S. Provisional Application Serial No. 06/256,269 filed Dec. 15, 2000; U.S. Provisional Application No. 60/296,581, filed Jun. 6, 2001; U.S. Provisional 60/296,580, filed Jun. 6, 2001; and U.S. Provisional Application No. (to be assigned), filed Oct. 19, 2001, attorney reference number 346393001500 entitled “Compositions and Methods for the Prevention and Treatment of Tissue Ischemia”, all hereby incorporated herein in their entirety.

### TECHNICAL FIELD

This invention generally relates to compositions and methods comprising gamma-tocopherol and/or a metabolite and/or a derivative thereof; beta-tocopherol and/or a metabolite and/or a derivative thereof; delta-tocopherol and/or a metabolite and/or derivative thereof; and/or a flavonoid and/or a derivative thereof, for treating and/or ameliorating the symptoms of tissue ischemia in a mammalian subject. The invention also relates to methods of making such compositions.

### BACKGROUND ART

Ischemia may be defined as the loss of blood flow to a tissue. Cerebral ischemia, also known as stroke, is the interruption or reduction of blood flow in the arteries feeding the brain. Loss of blood flow to a particular vascular region is known as focal ischemia; loss of blood flow to the entire brain, global ischemia. When deprived of blood, and thus, oxygen and glucose, brain tissue may undergo ischemic necrosis or infarction. The metabolic events thought to underlie such cell degeneration and death include: energy failure through ATP depletion; cellular acidosis; glutamate release; calcium ion influx; stimulation of membrane phospholipid degradation and subsequent free-fatty-acid accumulation; and free radical generation.

Myocardial ischemia occurs when the heart muscle does not receive an adequate blood supply and is thus deprived of necessary levels of oxygen and nutrients. A common cause of myocardial ischemia is atherosclerosis, which causes blockages in the blood vessels (coronary arteries) that provide blood flow to the heart muscle. Congestive heart failure (CHF) can also result in myocardial infarction.

Ischemic events affecting the intestines play a major role of the mortality and morbidity or numerous patients. As described in U.S. Pat. No. 6,191,109, ischemic injury to the small intestine leads to mucosol destruction, bacterial translocation and perforation.

Spinal cord injury is the most serious complication of spinal column trauma and also of operations on the aorta for treatment of thoracic and thoracoabdominal aneurysms (Kouchoukos, J. Thorac. Cardiovasc. Surg. 99:659-664, (1990)). As described in U.S. Pat. No. 5,648,331, the spinal cord is the organ most sensitive to ischemia during cross-clamping of the aorta, where the resultant injury may produce paraparesis or paraplegia. Spinal cord ischemia and paraplegia develop in approximately eleven percent (11%) of patients undergoing elective descending thoracic and thoracoabdominal aneurysm repair and nearly forty percent (40%) undergoing emergent repairs (Crawford, J. Vas. Surg. 3:389-402, (1986)).

Age-related macular degeneration (AMD) is the leading cause of visual impairment and blindness in the United States and elsewhere among people 65 years or older. Oxidative damage to the retina may be involved in the pathogenesis of AMD. As reported by Archives of Ophthalmology, vol. 119, 2001 in AREDS Report No. 8, at present, there is no proven treatment that slows or prevents the development of advanced AMD. U.S. Pat. No. 6,218,436 and WO 01/19383 A1 relate to ocular health.

Reactive oxygen species (ROS), also designated free radicals, include among other compounds singlet oxygen, the superoxide anion (O₂.⁻), nitric oxide (NO.), and hydroxyl radicals. Mitochondria are particularly susceptible to damage included by ROS, as these are generated continuously by the mitochondrial respiratory chain. Production of ROS increases when cells experience a variety of stresses, including organ ischemia and reperfusion, ultraviolet light exposure and other forms of radiation. Reiter et al. (1998) Ann. N.Y. Acad. Sci. 854:410-424; Saini et al. (1998) Res. Comm. Mol. Pathol. Pharmacol. 101:259-268; Gebicki et al. (1999) Biochem. J. 338:629-636. ROS are also produced in response to cerebral ischemia, including that caused by stroke, traumatic head injury and spinal injury. In addition, when metabolism increases or a body is subjected to extreme exercise, the endogenous antioxidant systems are overwhelmed, and free radical damage can take place. Free radicals are reported to cause the tissue-damage associated with some toxins and unhealthful conditions, including toxin-induced liver injury. Obata (1997) J. Pharm. Pharmacol. 49:724-730; Brent et al. (1992) J. Toxicol. Clin. Toxicol. 31:173-196; Rizzo et al. (1994) Zentralbl. Veterinarmed. 41:81-90; Lecanu et al. (1998) Neuroreport 9:559-663.

Vitamin E (alpha-tocopherol) and closely related compounds have long been thought to act as antioxidants. Halliwell (1996) Ann. Rev. Nutr. 16:33-50; Diplock et al. (1998) Br. J. Nutr. 80: S77-112. Alpha-tocopherol can prevent peroxidation in vitro, and this function can be replaced by other antioxidants. However, additional functions for vitamin E seem likely, since other antioxidants cannot relieve all the symptoms of vitamin E deficiency. There is increasing evidence that tocopherols are involved in the control of cell proliferation and differentiation. Traber et al. (1995) Am. J. Clin. Nutr. 62: 1501S-1509S. Alpha-tocopherol also functions as a scavenger of active nitrogen species (Halliwell et al. (1992) FEBS Lett. 313:62-66) and a gamma-tocopherol metabolite is an alleged natriuretic (Wechter et al. (1996) Proc. Natl. Acad. Sci. USA 93:6002-6007). See also U.S. Pat. Nos. 6,150,402; 6,083,982; 6,048,891, and 6,242,479. Alpha-tocopherol has been alleged to have an effect on cerebral ischemia. Yamamoto et al., 1983, Stroke, vol. 14:977-982; Hara et al., 1990, Brain Research, vol. 510: 335-338; and Altura, et al., 1996, Neuroscience Letters, vol. 220:207-210. In addition, alpha-tocopherol or its quinone derivatives may be involved in fatty acid desaturation. Infante (1986) Mol. Cell. Biochem. 69:93-108; Infante et al. (1998) FEBS Lett. 431:1-6. An additional major role of dietary alpha-tocopherol may be as a precursor of its D-alpha-tocopherolquinone metabolite whose semiquinone radical is required as an essential enzyme cofactor by carnitine-dependent, channeled mitochondrial fatty acid desaturases. Infante (1999) FEBS Lett. 446:1-5.

Tocopherols, while generally similar in overall chemical structure, may vary in biological function. Alpha-tocopherol is generally considered the most biologically active form of vitamin E; it is also the most abundant in adult human serum. Neuzil et al. (1998) Card. Drugs. Ther. 12:421-423; Strohschein et al. (1998) Anal. Chem. 70:13-18; Gonzalez (1990) Med. Hypothes. 32:107-110.

Among the compounds identified with antioxidant activity are some belonging to a group of naturally occurring phenylchromones known as flavonoids. Flavonoids, found in fruits, vegetables, grains, bark, roots, stems, flowers, tea and wine, are important to the flavor and color of their sources. For a review, see Croft (1998), p. 435-442, in Towards Prolongation of the Healthy Life Span, ed. Harman et al., Annals of the New York Academy of Sciences, New York. Evidence that diets rich in fruits and vegetables appear to protect against cardiovascular disease and some forms of cancer has lead to an interest in the biological effects of flavonoids.

Flavonoids are polyphenolic substances based on a flavan nucleus, comprising 15 carbon atoms, arranged in three rings as C₆-C₃-C₆. Flavonoids are biosynthetically derived from acetate and shikimate such that the A ring has a characteristic hydroxylation pattern at the 5 and 7 position. The B ring is usually 4′, 3′4′, or 3′4′5′-hydroxylated. Flavonoids have generally been classified into 12 different subclasses by the state of oxidation and the substitution pattern at the C2-C3 unit. These subclasses include flavanones (found in citrus fruits), flavones, flavonols (e.g., quercetin; found in onions, olives, tea, wine and apples), anthocyanidins (found in cherries, strawberries, grapes and colored fruits), chalcones, dihydrochalcones, aurones, flavanols, dihydroflavonols, proanthocyanidins (flavan-3,4-diols), isoflavones and neoflavones.

There remains a need for identification of effective compositions and methods which aid in the survival and recovery of cells during injury associated with a tissue ischemia or for mammalian subjects at risk for injury associated with a tissue ischemia.

The disclosure of all patents and publications cited herein are incorporated by reference in their entirety.

### DISCLOSURE OF THE INVENTION

The present invention relates to compositions and methods for treating and/or ameliorating the symptoms of tissue ischemia in a mammalian subject. The present invention also relates to compositions and methods for treating and/or ameliorating the symptoms of cerebral ischemia in a mammalian subject. Accordingly, the present invention provides methods for treating and/or ameliorating the symptoms of a cerebral ischemic condition in a mammalian subject, comprising administering to the subject an effective amount of a non-alpha tocopherol enriched tocopherol composition, and by said administering, reducing neuronal damage related to said cerebral ischemic condition. In some embodiments, the non-alpha tocopherol enriched tocopherol composition is a gamma-tocopherol enriched tocopherol composition. In additional embodiments, the non-alpha tocopherol is a gamma-tocopherol metabolite enriched composition. In other embodiments, the non-alpha tocopherol enriched tocopherol composition is a beta-tocopherol enriched tocopherol composition. In further embodiments, the non-alpha tocopherol is a beta-tocopherol metaoblite enriched composition. In further embodiments, the non-alpha tocopherol enriched tocopherol composition is a delta-tocopherol enriched tocopherol composition. In yet further embodiments, a non-alpha tocopherol is a delta-tocopherol metabolite enriched composition. In additional embodiments, the cerebral ischemic condition is secondary to an occlusion of the cerebral vasculature. In further embodiments, the occlusion is due to a thromboembolis. In additional embodiments, the occlusion is due to a spasm of the coronary vasculature. In further embodiments, the cerebral ischemic condition is secondary to a cessation of cardiac function. In further embodiments, the cerebral ischemic condition is secondary to a cardiopulmonary bypass procedure. In additional embodiments, the cerebral ischemic condition is secondary to a hemorrhagic event in the cerebral vasculature. In some embodiments, a non-alpha tocopherol composition comprises a non-alpha tocopherol in a range of about 1 to about 1000 mg per kg body weight of said mammalian subject. In some embodiments, a non-alpha tocopherol composition comprises a non-alpha tocopherol in a range of about 1 to about 50 mg per kg body weight of said mammalian subject. In other aspects the present invention provides gamma-tocopherol enriched tocopherol compositions comprising gamma tocopherol in an amount effective to reduce neuronal damage related to a cerebral ischemic condition. In further embodiments, the present invention provides beta-tocopherol enriched tocopherol compositions comprising beta tocopherol in an amount effective to reduce neuronal damage related to a cerebral ischemic condition. In yet further embodiments, the present invention provides delta-tocopherol enriched tocopherol compositions comprising delta tocopherol in an amount effective to reduce neuronal damage related to a cerebral ischemic condition.

The present invention provides a method for treating and/or ameliorating the symptoms of a tissue ischemic condition in a mammalian subject, comprising administering to the subject an effective amount of a beta-tocopherol enriched tocopherol composition, and by said administering, reducing tissue damage related to said tissue ischemic condition. The present invention also provides a method for treating and/or ameliorating the symptoms of a tissue ischemic condition in a mammalian subject, comprising administering to the subject an effective amount of a beta-tocopherol metabolite enriched composition, and by said administering, reducing tissue damage related to said tissue ischemic condition. In some embodiments, the tissue ischemic condition is selected from the group consisting of cerebral ischemia; intestinal ischemia; spinal cord ischemia; cardiovascular ischemia; myocardial ischemia associated with myocardial infarction; mycardial ischemia associated with CHF, ischemia associated with age-related macular degeneration (AME); liver ischemia; kidney ischemia; dermal ischemia; vasoconstriction-induced tissue ischemia; penile ischemia as a consequence of priapism; ischemia associated with thromboembolytic disease; ischemia associated with microvascular disease; and ischemia associated with diabetic ulcers, gangrenous conditions, post-trauma syndrome, cardiac arrest resuscitation, peripheral nerve damage or neuropathies. In some embodiments, the tissue ischemic condition is cerebral ischemia. In further embodiments, a composition comprises a beta-tocopherol in a range of about 1 to about 1000 mg per kg body weight of said mammalian subject. In additional embodiments, a composition comprises a beta-tocopherol in a range of about 1 to about 50 mg per kg body weight of said mammalian subject. In yet additional embodiments, a composition comprises a beta-tocopherol in a range of about 10 to about 100 mg per kg body weight of said mammalian subject.

The present invention also provides a method for treating and/or ameliorating the symptoms of a tissue ischemic condition in a mammalian subject, comprising administering to the subject an effective amount of a delta-tocopherol enriched tocopherol composition, and by said administering, reducing tissue damage related to said tissue ischemic condition. The present invention also provides a method for treating and/or ameliorating the symptoms of a tissue ischemic condition in a mammalian subject, comprising administering to the subject an effective amount of a delta-tocopherol metabolite enriched composition, and by said administering, reducing tissue damage related to said tissue ischemic condition. In some embodiments, the tissue ischemic condition is selected from the group consisting of cerebral ischemia; intestinal ischemia; spinal cord ischemia; cardiovascular ischemia; myocardial ischemia associated with myocardial infarction; mycardial ischemia associated with CHF, ischemia associated with age-related macular degeneration (AMD); liver ischemia; kidney ischemia; dermal ischemia; vasoconstriction-induced tissue ischemia; penile ischemia as a consequence of priapism; ischemia associated with thromboembolytic disease; ischemia associated with microvascular disease; and ischemia associated with diabetic ulcers, gangrenous conditions, post-trauma syndrome, cardiac arrest resuscitation, peripheral nerve damage or neuropathies. In further embodiments, the tissue ischemic condition is cerebral ischemia. In further embodiments, a composition comprises a delta-tocopherol in a range of about 1 to about 1000 mg per kg body weight of said mammalian subject. In additional embodiments, a composition comprises a delta-tocopherol in a range of about 1 to about 50 mg per kg body weight of said mammalian subject. In yet further embodiments, a composition comprises a delta-tocopherol in a range of about 10 to about 100 mg per kg body weight of said mammalian subject.

The present invention also provides a method for treating and/or ameliorating the symptoms of a non-cardiovascular tissue ischemic condition in a mammalian subject, comprising administering to the subject an effective amount of a gamma-tocopherol enriched tocopherol composition, and by said administering, reducing tissue damage related to said non-cardiovascular tissue ischemic condition. The present invention also provides a method for treating and/or ameliorating the symptoms of a non-cardiovascular tissue ischemic condition in a mammalian subject, comprising administering to the subject an effective amount of a gamma-tocopherol metabolite enriched composition, and by said administering, reducing tissue damage related to said non-cardiovascular tissue ischemic condition. In some embodiments, the tissue ischemic condition is selected from the group consisting of intestinal ischemia; spinal cord ischemia; ischemia associated with age-related macular degeneration (AMD); liver ischemia; kidney ischemia; dermal ischemia; vasoconstriction-induced tissue ischemia; penile ischemia as a consequence of priapism; ischemia associated with thromboembolytic disease; ischemia associated with microvascular disease; and ischemia associated with diabetic ulcers, gangrenous conditions, post-trauma syndrome, peripheral nerve damage or neuropathies. In some embodiments, a composition comprises gamma-tocopherol in a range of about 1 to about 1000 mg per kg body weight of said mammalian subject. In further embodiments, a composition comprises gamma-tocopherol in a range of about 1 to about 50 mg per kg body weight of said mammalian subject. In yet additional embodiments, a composition comprises gamma-tocopherol in a range of about 10 to about 100 mg per kg body weight of said mammalian subject.

The present invention also provides a method for treating and/or ameliorating the symptoms of a tissue ischemic condition in a mammalian subject, comprising administering to the subject an effective amount of a composition comprising a flavonoid and/or a flavonoid derivative, and by said administering, reducing tissue damage related to said tissue ischemic condition, wherein said flavonoid specifically excludes diosmin and hesperidin. In some embodiments, the tissue ischemic condition is selected from the group consisting of intestinal ischemia; spinal cord ischemia; ischemia associated with age-related macular degeneration (AMD); liver ischemia; kidney ischemia; dermal ischemia; vasoconstriction-induced tissue ischemia; penile ischemia as a consequence of priapism; ischemia associated with thromboembolytic disease; ischemia associated with microvascular disease; and ischemia associated with diabetic ulcers, gangrenous conditions, post-trauma syndrome, peripheral nerve damage or neuropathies. In further embodiments, the flavonoid is chelated to a metal. In other embodiments, the metal is Fe. In yet additional embodiments, the flavonoid is selected from the group consisting of chrysin, daidzein, hesperetin, luteolin, quercetin, bromoquercetin, rutin, and biochanin. In further embodiments, the composition comprises two flavonoids. In yet additional embodiments, the flavonoid is quercetin, hesperetin, or bromoquercetin. In further embodiments, the composition comprises quercetin and hesperetin. In some embodiments, composition comprises a flavonoid in a range of about 1 to about 1000 mg per kg body weight of said mammalian subject. In additional embodiments, a composition comprises a flavonoid in a range of about 1 to about 25 mg per kg body weight of said mammalian subject. In further embodiments, a composition comprises a flavonoid in a range of about 10 to about 100 mg per kg body weight of said mammalian subject.

The present invention also provides a method for treating and/or ameliorating the symptoms of a tissue ischemic condition in a mammalian subject, comprising administering to the subject an effective amount of a composition comprising a non-alpha tocopherol and a flavonoid, wherein said flavonoid is selected from the group consisting of chrysin, daidzein, diosmin, hesperetin, hesperidin, luteolin, quercetin, bromoquercetin, rutin and biochanin, and by said administering, reducing tissue damage related to said tissue ischemic condition. In some embodiments, the tissue ischemic condition is cerebral ischemia. In other embodiments, the tissue ischemic condition is cardiovascular ischemia. In further embodiments, the non-alpha tocopherol is selected from the group consisting of gamma-tocopherol, beta-tocopherol, delta-tocopherol, a gamma-tocopherol metabolite, a beta-tocopherol metabolite, and a delta-tocopherol metabolite. In further embodiments, the non-alpha tocopherol is gamma-tocopherol. In yet additional embodiments, the non-alpha-tocopherol is a gamma-tocopherol metabolite. In further embodiments, the composition comprises gamma-tocopherol and two flavonoids. In yet further embodiments, the composition comprises gamma-tocopherol, quercetin and hesperetin. In yet additional embodiments, the flavonoid is metal chelated. In some embodiments, the composition comprises gamma-tocopherol in the range of about 1 to about 1000 mg/kg body weight of mammalian subject, hesperetin in the range of about 1 to about 1000 mg/kg body weight of mammalian subject and quercetin in a range of about 1 to about 1000 mg/kg body weight of mammalian subject. In yet additional embodiments, the composition comprises gamma-tocopherol in the range of about 1 to about 50 mg/kg body weight of mammalian subject, hesperetin in the range of about 1 to about 25 mg/kg body weight of mammalian subject and quercetin in a range of about 1 to about 25 mg/kg body weight of mammalian subject. In further embodiments, the composition comprises gamma-tocopherol in the range of about 10 to about 100 mg/kg body weight of mammalian subject, hesperetin in the range of about 10 to about 100 mg/kg body weight of mammalian subject and quercetin in a range of about 10 to about 100 mg/kg body weight of mammalian subject.

In some aspects of the present invention, a gamma tocopherol enriched tocopherol composition comprises at least 50%, at least 60%, at least 70%, at least 80%, at least 90%, at least 95% gamma-tocopherol, or at least 98% gamma-tocopherol. In other aspects, a beta-tocopherol enriched tocopherol composition comprises at least 50%, at least 60%, at least 70%, at least 80%, at least 90%, at least 95% beta-tocopherol, or at least 98% beta-tocopherol. In yet other aspects, a delta-tocopherol enriched tocopherol composition comprises at least 50%, at least 60%, at least 70%, at least 80%, at least 90%, at least 95% delta-tocopherol, or at least 98% delta-tocopherol. In further aspects, a gamma tocopherol metabolite enriched composition comprises at least 50%, at least 60%, at least 70%, at least 80%, at least 90%, at least 95%, or at least 98% gamma-tocopherol metabolite. In some embodiments, the gamma-tocopherol metabolite is gamma-CEHC. In other aspects, a beta-tocopherol metabolite enriched composition comprises at least 50%, at least 60%, at least 70%, at least 80%, at least 90%, at least 95%, or at least 98% beta-tocopherol metabolite. In yet other aspects, a delta-tocopherol metabolite enriched composition comprises at least 50%, at least 60%, at least 70%, at least 80%, at least 90%, at least 95%, or at least 98% delta-tocopherol metabolite.

The present invention also provides compositions comprising a non-alpha tocopherol and a flavonoid in amounts effective to reduce neuronal damage related to a cerebral ischemic condition. In some embodiments, the composition comprises hesperetin or quercetin. In other embodiments, the composition comprises gamma-tocopherol, hesperetin or quercetin. In yet further embodiments, the composition comprises diosmin and gamma-tocopherol. In yet further embodiments, the composition comprises delta-tocopherol and hesperetin. In yet further embodiments, the composition comprises gamma-tocopherol and biochanin A.

The present invention also encompasses novel compositions and methods for making such compositions. In some embodiments of the present invention, a composition is a nutritional composition. In other embodiments, a composition is a pharmaceutical composition. In some embodiments, the administering is via an enteral route. In other embodiments, the administering is via an oral route. In yet further embodiments, the administering is via a parenteral route.

### BRIEF DESCRIPTION OF THE DRAWINGS

FIG. 1 shows the effect of gamma-tocopherol and its metabolite, gamma-carboxy ethyl hydroxy chroman (gamma-CEHC), on the volumetric comparison of total infarct with administration of gamma-tocopherol and gamma-CEHC at the time of Middle Cerebral Artery Occlusion (MCAO) as described in Example 2.

FIG. 2 shows the effect of gamma-tocopherol and its metabolite, gamma-CEHC, on the volumetric comparison of total infarct with administration of gamma-tocopherol and gamma-CEHC at reperfusion as described in Example 2.

FIG. 3 illustrates 5 general formulas of tocopherol metabolites.

FIG. 4 shows the effect of the flavonoid, bromo-quercetin on infarct size in a myocardial ischemia model.

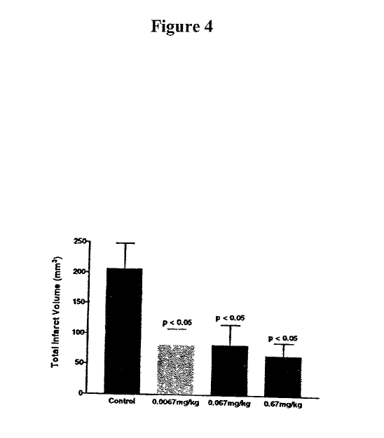

FIG. 5 shows the effect of gamma-tocopherol on stroke protection as measured in a cerebral ischemia MCAO model.

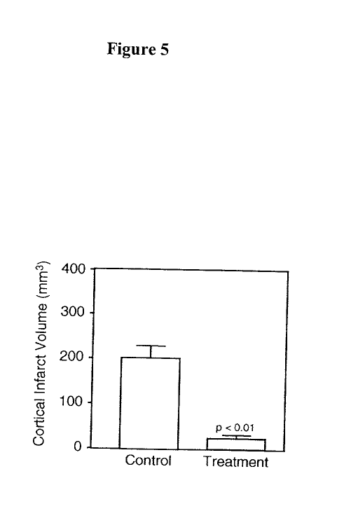

FIG. 6 shows the general structure of a flavonoid and positions of functional groups that can be added to provide increased activity to the molecule.

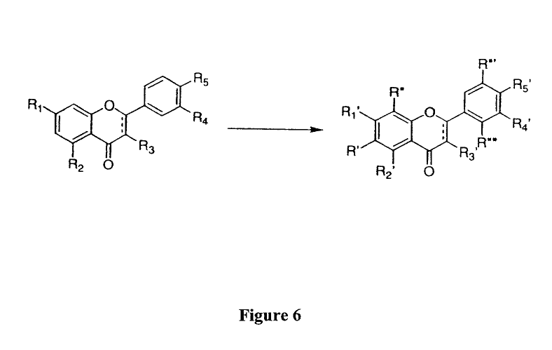

FIG. 7 shows the effects of addition of increasing concentrations of Fe(III) on flavonoid protection of oxidatively stressed, energetically competent cells.

### MODES FOR CARRYING OUT THE INVENTION

The present invention generally relates to naturally-occurring compounds as well as synthetic derivatives of naturally occurring compounds and non-naturally occurring mixtures of naturally-occurring compounds that can be used in nutritional and pharmaceutical compositions that are protective in tissue ischemia, such as cerebral ischemia (stroke) and mycardial injury subsequent to ischemia or hypoxia. The present invention provides compositions and methods for treating and/or ameliorating the symptoms of tissue ischemia, such as for example, cerebral ischemia, cardiovascular ischemia, spinal cord ischemia, intestinal ischemia, liver ischemia, kidney ischemia, dermal ischemia, and other tissue ischemias by for example reducing tissue or cell death and/or reducing tissue edema associated with the ischemic event, or reducing other symptoms or conditions associated with the tissue ischemia, such as, for example, by reducing infarct size associated with myocardial ischemia. The present invention also provides compositions and methods for treating and/or ameliorating the symptoms of cerebral ischemia, such as for example, by reducing neuronal cell death, reducing tissue edema, and/or reducing cognitive dysfunction associated with a cerebral ischemic disorder or reducing other symptoms and/or conditions associated with a cerebral ischemic condition, such as, for example, reducing infarct size, tissue edema or cognitive disorder associated with the presence of micro-emboli or a hypoxic condition.

The present invention provides gamma-tocopherol enriched tocopherol compositions, beta-tocopherol enriched tocopherol compositions and delta-tocopherol enriched compositions and methods for using such compositions. In preferred embodiments, gamma-tocopherol enriched tocopherol compositions of the present invention comprise at least 50% gamma-tocopherol, at least 55% gamma-tocopherol, at least 60% gamma-tocopherol, at least 65% gamma-tocopherol, at least 70% gamma-tocopherol, at least 75% gamma-tocopherol, at least 80% gamma-tocopherol, at least 85% gamma tocopherol, at least 90% gamma-tocopherol and at least 95% gamma-tocopherol. Gamma-tocopherol enriched tocopherol compositions comprise less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 5% alpha-tocopherol. In some embodiments, a gamma-tocopherol “enriched” tocopherol composition comprises gamma-tocopherol as the sole active ingredient. As used herein, an “active ingredient” is one that is able to treat and/or prevent and/or ameliorate the symptoms of a tissue ischemia in a mammalian subject. In preferred embodiments, an active ingredient is able to reduce cell or tissue damage associated with tissue ischemia at least about 30%, at least about 40%, at least about 50%, at least about 60%, at least about 70%, at least about 80%, at least about 80%, and even more preferably at least about 90%, in experimental models such as those described herein. In additional embodiments, a non-alpha tocopherol enriched tocopherol composition comprises a non-alpha tocopherol in an amount effective to reduce cell death, reduce infarct size, reduce tissue edema associated with the ischemic condition, and/or in cerebral ischemia, reduce cognitive dysfunction and may further comprise a gamma-tocopherol metabolite and/or derivative and may further comprise additional tocopherols and/or other ingredients.

In other embodiments, the gamma-tocopherol enriched tocopherol compositions of the present invention comprise additional active ingredients, and/or additional non-tocopherols. In some embodiments of gamma-tocopherol enriched tocopherol compositions, the gamma-tocopherol and additional ingredient(s) provide a synergistic effect. Gamma-tocopherol and an additional ingredient are considered to be synergistic when their combined effect is greater than additive of the individual effects. In some embodiments of the present invention, a synergist is selected from the group flavonoids. In some embodiments, the composition comprises a non-alpha tocopherol and a flavonoid selected from the group hesperetin, quercetin, diosmin and biochanin A. In other embodiments, the composition comprises gamma-tocopherol, hesperetin or quercetin. In yet further embodiments, the composition comprises diosmin and gamma-tocopherol. In yet further embodiments, the composition comprises delta -tocopherol and hesperetin. In yet further embodiments, the composition comprises gamma-tocopherol and biochanin A.

In preferred embodiments, beta-tocopherol enriched tocopherol compositions of the present invention comprise at least 50% beta-tocopherol, at least 55% beta-tocopherol, at least 60% beta-tocopherol, at least 65% beta-tocopherol, at least 70% beta-tocopherol, at least 75% beta-tocopherol, at least 80% beta-tocopherol, at least 85% beta tocopherol, at least 90% beta-tocopherol and at least 95% beta-tocopherol . Beta-tocopherol enriched tocopherol compositions comprise less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 5% alpha-tocopherol. In some embodiments, beta-tocopherol enriched tocopherol compositions comprise beta-tocopherol as the sole active ingredient. In additional preferred embodiments, a beta-tocopherol enriched tocopherol composition comprises beta-tocopherol in an amount effective to reduce cell death, reduce infarct size, reduce tissue edema associated with the ischemic condition, and/or reduce cognitive dysfunction, such as in cerebral ischemia and may further comprise a beta-tocopherol metabolite and/or derivative and may further comprise additional tocopherols and/or other ingredients. In other preferred embodiments, the beta-tocopherol enriched tocopherol compositions of the present invention comprise additional active ingredients, and/or additional non-tocopherols. In some embodiments of beta-tocopherol enriched tocopherol compositions, the beta-tocopherol and additional ingredient(s) provide a synergistic effect. Beta-tocopherol and an additional ingredient are considered to be synergistic when their combined effect is greater than additive of the individual effects.

In preferred embodiments, delta-tocopherol enriched tocopherol compositions of the present invention comprise at least 50% delta-tocopherol, at least 55% delta-tocopherol, at least 60% delta-tocopherol, at least 65% delta-tocopherol, at least 70% delta-tocopherol, at least 75% delta-tocopherol, at least 80% delta-tocopherol, at least 85% delta tocopherol, at least 90% delta-tocopherol and at least 95% delta-tocopherol. Delta-tocopherol enriched tocopherol compositions comprise less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 5% alpha-tocopherol. In some embodiments, delta-tocopherol enriched tocopherol compositions comprise delta-tocopherol as the sole active ingredient. In additional preferred embodiments, a delta-tocopherol enriched tocopherol composition comprises delta-tocopherol in an amount effective to reduce cell death, reduce infarct size, reduce tissue edema associated with the ischemic condition, and/or reduce cognitive dysfunction, such as for cerebral ischemia and may further comprise a delta-tocopherol metabolite and/or derivative and may further comprise additional tocopherols and/or other ingredients. In other preferred embodiments, the delta-tocopherol enriched tocopherol compositions of the present invention comprise additional active ingredients, and/or additional non-tocopherols. In some embodiments of delta-tocopherol enriched tocopherol compositions, the delta-tocopherol and additional ingredient(s) provide a synergistic effect. Delta-tocopherol and an additional ingredient are considered to be synergistic when their combined effect is greater than additive of the individual effects.

Assays for measuring the effect of gamma-tocopherol enriched tocopherol compositions, beta-tocopherol enriched compositions and delta-tocopherol compositions are provided herein and are known to those of skill in the art.

In other aspects, the present invention provides gamma-tocopherol metabolite or derivative enriched compositions, beta-tocopherol metabolite or derivative enriched compositions, delta-tocopherol metabolite or derivative enriched compositions, and flavonoid enriched compositions, and methods for using such compositions. In preferred embodiments, the gamma-tocopherol, beta-tocopherol or delta-tocopherol metabolite enriched compositions of the present invention comprise at least 50% gamma-, beta-, or delta-tocopherol metabolite or derivative, at least 55% gamma-, beta-, or delta-tocopherol metabolite or derivative, at least 60% gamma-, beta-, or delta-tocopherol metabolite or derivative, at least 65% gamma-, beta-, or delta-tocopherol metabolite or derivative, at least 70% gamma-, beta-, or delta-tocopherol metabolite or derivative, at least 75% gamma-, beta-, or delta-tocopherol metabolite or derivative, at least 80% gamma-, beta-, or delta-tocopherol metabolite or derivative, at least 85% gamma-, beta-, or delta-tocopherol metabolite or derivative, at least 90% gamma-, beta-, or delta-tocopherol metabolite or derivative and at least 95% gamma-, beta-, or delta-tocopherol metabolite or derivative. Gamma-tocopherol, beta-tocopherol or delta-tocopherol metabolite enriched compositions comprises less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 5% alpha-tocopherol. In some embodiments, gamma-, beta-, or delta-tocopherol metabolite or derivative enriched compositions comprise gamma-, beta-, or delta-tocopherol metabolite or derivative as the sole active ingredient. In additional preferred embodiments, a gamma-, beta-, or delta-tocopherol metabolite or derivative enriched composition comprises the gamma-, beta-, or delta-tocopherol metabolite or derivative in an amount effective to reduce cell death, reduce infarct size, reduce tissue edema associated with the ischemic condition, and/or reduce cognitive dysfunction, such as in cerebral ischemia. A gamma-, beta-, or delta-tocopherol metabolite or derivative enriched composition may further comprise tocopherol(s). In other preferred embodiments, the gamma-tocopherol, beta-tocopherol or delta-tocopherol metabolite or derivative enriched compositions of the present invention comprise additional active ingredients, and/or additional non-tocopherols. In some embodiments of gamma-, beta-, or delta-tocopherol metabolite or derivative enriched compositions, the gamma-, beta-, or delta-tocopherol metabolite or derivative and additional ingredient(s) provide a synergistic effect. A gamma-, beta-, or delta-tocopherol metabolite or derivative and an additional ingredient are considered to be synergistic when their combined effect is greater than additive of the individual effects. Assays for measuring the effect of gamma-, beta-, or delta-tocopherol metabolite or derivative enriched compositions are provided herein and are known to those of skill in the art.

In illustrative embodiments disclosed herein a gamma-tocopherol enriched tocopherol composition and a gamma-tocopherol metabolite enriched composition are shown to reduce total infarct at the time of MCAO and at the time of reperfusion, as described in Example 2. In further illustrative embodiments shown herein, a beta-tocopherol enriched tocopherol composition is shown to reduce total infarct size at three hours pre-MCAO when administered gavage and a delta-tocopherol enriched composition is shown to reduce total infarct size at the time of MCAO when administered IV.

Definitions

“Tissue Ischemia” or “tissue ischemic” or “a tissue ischemic condition” refer to a medical event which is pathological in origin, or to a surgical intervention which is imposed on a subject, wherein circulation to a region of the tissue is impeded or blocked, either temporarily, as in vasospasm or transient ischemic attach (TIA) in cerebral ischemia or permanently, as in thrombolic occlusion in cerebral ischemia. The affected region is deprived of oxygen and nutrients as a consequence of the ischemic event. This deprivation leads to the injuries of infarction or in the region affected. The present invention encompasses cerebral ischemia; intestinal ischemia; spinal cord ischemia; cardiovascular ischemia; ischemia associated with CHF, liver ischemia; kidney ischemia; dermal ischemia; vasoconstriction-induced tissue ischemia, such as a consequence of Raynaud's disorder; penile ischemia as a consequence of priapism; and ischemia associated with thromboembolytic disease; microvascular disease; such as for example diabetes and vasculitis; diabetic ulcers; gangrenous conditions; post-trauma syndrome; cardiac arrest resuscitation; and peripheral nerve damage and neuropathies; and other ischemias, including ischemia associated with ocular health concerns, such as for example, age-related macular degeneration (AMD). Ischemia occurs in the brain during, for example, a stroke, cardiac arrest, severe blood loss due to injury or internal hemorrhage and other similar conditions that disrupt normal blood flow. Ischemia occurs in myocardial tissue as a result of, for example, atherosclerosis and CHF. It may also occur after a trauma to the tissue since the pressure caused by edema presses against and flattens the arteries and veins inside the tissue, thereby reducing their ability to carry blood through the tissue. Cerebral ischemia may also occur as a result of macro-or micro-emboli, such as may occur subsequent to cardiopulmonary bypass surgery. Age-related macular degeneration may be associated with oxidative damage to the retina as a result of an ischemic condition. As used herein, a “non-cardiovascular” ischemic condition specifically excludes an ischemic condition of the cardio-pulmonary system or circulatory system. As used herein, a “non-cerebral” ischemic condition specifically excludes an ischemic condition of the brain.

“Cerebral Ischemia” or “cerebral ischemic” or “a cerebral ischemic condition” refer to a medical event which is pathological in origin, or to a surgical intervention which is imposed on a subject, wherein circulation to a region of the brain is impeded or blocked, either temporarily, as in vasospasm or transient ischemic attach (TIA) or permanently, as in thrombolic occlusion. The affected region is deprived of oxygen and nutrients as a consequence of the ischemic event. This deprivation leads to the injuries of infarction or in the region affected. Ischemia occurs in the brain during, for example, a thromboembolic stroke, hemorrhagic stroke, cerebral vasospasm, head trauma, cardiac arrest, severe blood loss due to injury or internal hemorrhage and other similar conditions that disrupt normal blood flow. It may also occur after a head trauma, since the pressure caused by edema presses against and flattens the arteries and veins inside the brain, thereby reducing their ability to carry blood through the brain. Cerebral ischemia may also occur as a result of macro-or micro-emboli, such as may occur subsequent to cardiopulmonary bypass surgery.

By “tocopherol” is meant any of a family of molecules (including both tocopherols and tocotrienols and derivatives thereof) which are characterized by a 6-chromanol ring structure and a side chain at the 2 position. A “gamma-tocopherol enriched tocopherol composition”, “beta-tocopherol enriched tocopherol composition” or a “delta-tocopherol enriched tocopherol composition” as used herein refers to the particular tocopherol as being enriched with respect to total tocopherols in the composition. Tocopherols possess a 4′, 8′, 12′-trimethyltridecyl phytol side chain, and the tocotrienols differ by the presence of double bonds at the 3′, 7′ and 11′ positions of the side chain. As used herein, the term “tocopherol” encompasses, but is not limited to:

alpha-tocopherol, [2R-2R*(4R*,8R*)]-3,4-dihydro-2,5,7,8-tetramethyl-2-(4,8,12-trimethyltridecyl)-2H-1-benzopyran-6-ol; 2,5,7,8-tetramethyl-2-(4′,8′,12′-trimethyltridecyl)-6-chromanol; 5,7,8-trimethyltocol, Fernholz (1937) J. Am. Chem. Soc. 59:1154 and 60:700;

beta-tocopherol, 3,4-dihydro-2,5,8-trimethyl-2-(4,8,12-trimethyltridecyl)-2H-1-benzopyran-6-ol; 2,5,8-trimethyl-2-(4,8,12-trimethyltridecyl)-6-chromanol; 5-8-dimethyltocol; cumotocopherol; neotocopherol; p-xylotocopherol;

gamma-tocopherol, 3,4-dihydro-2,7,8-trimethyl-2-(4,8,12-trimethyltridecyl)-2H-1-benzyopyran-6-ol; 2,7,8-trimethyl-2-(4,8,12-trimethyltridecyl)-6-chromanol; 7,8-dimethyltocol; o-xylotocopherol;

delta-tocopherol, [2R-[2R*(4R*,8R*)]]-3,4-dihydro-2,8-dimethyl-2-(4,8,12-trimethyltridecyl)-2H-1-benzo-pyran-6-ol; 8-methyltocol;

epsilon-tocopherol, [R-(E,E)]-3,4-dihydro-2,5,8-trimethyl-2-(4,8,12-trimethyl-3,7,11-tridecatrienyl)-2H-1-benzopyran-6-ol; 2,5,8-trimethyl-2-(4,8,12-trimethyltrideca-3,7,11-trienyl)chroman-6-ol; 5-methyltocol;

zeta₁-tocopherol, 3,4-dihydro-2,5,7,8-tetramethyl-2-(4,8,12-trimethyl-3,7,11-tridecatrienyl)-2H-1-benzopyran-6-ol; 2,5,7,8-tetramethyl-2-(4,8,12-trimethyl-3,7,11-tridecatrienyl)-6-chromanol; 5,7,8-trimethyltocotrien-3′,7′,11′-ol;

zeta₂-tocopherol, 3,4-dihydro-2,5,7-trimethyl-2-(4,8,12-trimethyltridecyl)-2H-1-benzopyran-6-ol; 2,5,7-trimethyl-2-(4,8,12-trimethyltridecyl-6-chromanol; 5,7-dimethyltocol; and

eta-tocopherol, 3,4-dihydro-2,7-dimethyl-2-(4,8,12-trimethyltridecyl)-2H-1-benzopyran-6-ol; 2,7-dimethyl-2-(4,8,12-trimethyltridecyl)-6-chromanol; 7-methyltocol. See The Merck Index (1996), Twelfth Edition, Merck & Co., Whitehouse Station, N.J., pp. 1620-1621 and 1712, and references cited therein. Other tocopherols include xi₁-, xi₂-, and sigma-tocopherols.

Derivatives of these compounds include, but are not limited to, salts, including but not limited to succinate, nicotinate, allophanate, acetate, and phosphate salts of the tocopherols described herein. Salts also include pharmaceutically acceptable salts. Derivatives include halogenated derivatives. Derivatives also include quinone derivatives and prodrug forms of tocopherols, such as those described in U.S. Pat. No. 5,114,957. Additional tocopherols and derivatives thereof are described in, e.g., U.S. Pat. Nos. 5,606,080 and 5,235,073. Preparation of various tocopherols are described in, e.g., U.S. Pat. Nos. 5,504,220, 4,978,617, and 4,977,282. Various tocopherols are available from Sigma Chemical Co., St. Louis, Mo.

The term “γ-CEHC” refers to the 2,7,8-trimethyl-2-(β-carboxy-ethyl)-6-hydroxy chroman, having a molecular weight of 264. This compound is a metabolite of γ-tocopherol and its synthesis and properties are described in U.S. Pat. No. 6,083,982, incorporated herein by reference (where it is also referred to as “LLU-alpha”). γ-CEHC may be in the racemic form or as the S enantiomer. A general discussion of the isolation and characterization of γ-CEHC is provided by Wechter et al. (U.S. Pat. No. 6,150,402).

The term “γ-CEBC” refers to the compound the 2,7,8-trimethyl-2-(β-carboxy-butyl)-6-hydroxy chroman, having a molecular weight of about 278. It may also be present in racemic form or as pure isomer(s).

By “gamma-tocopherol derivative” is meant gamma.-tocopherol metabolites and synthetic chroman derivatives including, but not limited to, γ-CEHC, γ-CEBC, racemic chromans, chroman methyl esters, chroman esters, chroman amides, R.sub.4 chroman esters, oxidized chroman derivatives, racemic 2,5,7,8-tetramethyl-2-(.beta.-carboxyethyl)-6-hydroxy chroman, and the like. Additional derivatives were defined by Wechter (U.S. Pat. No. 6,083,982.

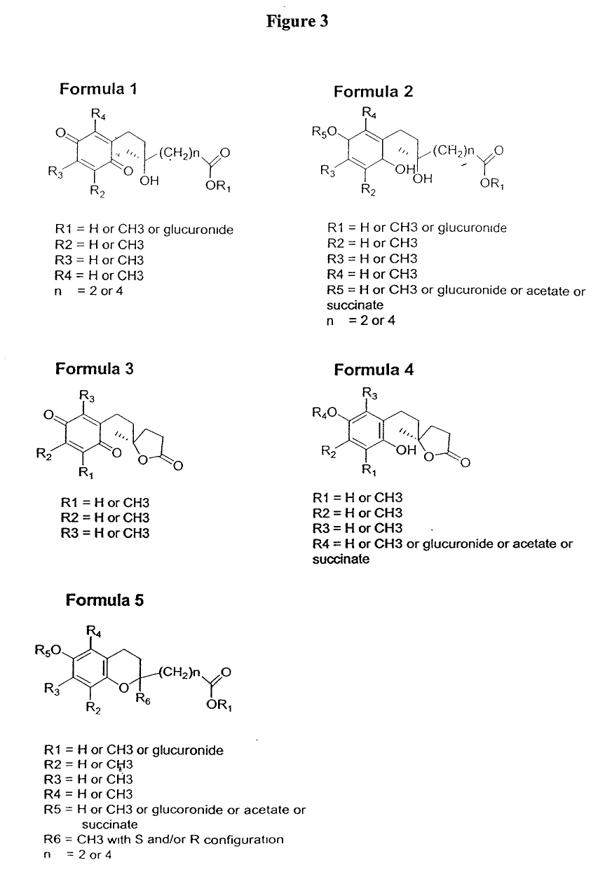

“Tocopherol metabolites and derivatives” include, for example, 2,5,7,8-tetramethyl-2-(.beta.-carboxyethyl)-chroman, 2,7,8-trimethyl-2-(.beta.-carboxyethyl) chroman, racemic 4-methyl-6-(5,6-dimethylbenzohinoyl)-4-hexanolid, 4-Methyl-6-(3,5,6-trimethylbenzochinoyl)4-hexanolid, (S)-4-Methyl-6-(5,6-dimethylbenzochinoyl)-4-hexanolid, 2,7,8-Trimethyl-2-(.beta.-carboxyethyl)-6-acetyl chroman, 2,7,8-Trimethyl-2-(.beta.-carboxyethyl)-6-acetyl chroman methyl ester, and benzodipyran methyl ester, as well as γ-CEHC and γ-CEBC, described above, and compounds shown in FIG. 3 herein. Other gamma.-tocopherol metabolites and synthetic chroman derivatives may be known by those of skill in the art or will be discovered in the future and are encompassed by this definition.

In the body of a subject, gamma-tocopherol, beta-tocopherol and delta-tocopherol break down into metabolites, including for example, the metabolites described in Wechter et al. U.S. Pat. Nos. 6,150,402; 6,083,982; 6,048,891 and 6,242,479, specifically incorporated herein in their entirety. In particular, the present invention encompasses the use of gamma-tocopherol enriched tocopherol compositions that further comprise gamma-tocopherol metabolites such as gamma-CEHC, racemic gamma-CEHC and (S) gamma-CEHC. The general structure of other tocopherol metabolites are shown in FIG. 3. The present invention also encompasses the use of tocopherol metabolite enriched compositions and such compositions that further comprise a tocopherol.

By a “non-alpha” tocopherol enriched tocopherol composition is meant a composition that is enriched in, i.e., that comprises 50% or greater, a tocopherol other than alpha-tocopherol. Examples of non-alpha tocopherol enriched tocopherol compositions include without limitation, beta-tocopherol enriched tocopherol compositions, beta-tocopherol metabolite enriched compositions, delta-tocopherol enriched tocopherol compositions, delta-tocopherol metabolite enriched compositions, gamma-tocopherol enriched tocopherol compositions, gamma-tocopherol metabolite enriched compositions, epsilon-tocopherol enriched tocopherol compositions, zeta-tocopherol enriched tocopherol compositions, etc. By a “non-gamma” tocopherol enriched composition is meant a composition that is enriched in a tocopherol other than gamma-tocopherol. Examples of non-gamma tocopherol enriched tocopherol compositions include without limitation, beta-tocopherol enriched tocopherol compositions, beta-tocopherol metabolite enriched compositions, delta-tocopherol enriched tocopherol compositions, delta-tocopherol metabolite enriched compositions, gamma-tocopherol enriched tocopherol compositions, gamma-tocopherol metabolite enriched compositions, epsilon-tocopherol enriched tocopherol compositions, zeta-tocopherol enriched tocopherol compositions, etc. A non-alpha, non-gamma-tocopherol enriched tocopherol composition refers to a composition that is enriched in a tocopherol other than alpha-tocopherol and gamma-tocopherol. Examples of non-alpha, non-gamma-tocopherol enriched tocopherol compositions include without limitation, beta-tocopherol enriched tocopherol compositions, beta-tocopherol metabolite enriched compositions, delta-tocopherol enriched tocopherol compositions, delta-tocopherol metabolite enriched compositions, gamma-tocopherol enriched tocopherol compositions, gamma-tocopherol metabolite enriched compositions, epsilon-tocopherol enriched tocopherol compositions, zeta-tocopherol enriched tocopherol compositions, etc.

By a “non-tocopherol” is meant any compound which is not a tocopherol, tocopherol metabolite, tocotrienol, or derivative thereof, or the like.

By “flavonoid” is meant any of a class of polyphenolic molecules based on a flavan nucleus, comprising 15 carbon atoms, arranged in three rings as C₆-C₃-C₆. Flavonoids are generally classified into subclasses by the state of oxidation and the substitution pattern at the C2-C3 unit. As used herein, the term “flavonoid” encompasses, but are not limited to, flavanones, flavonols, flavones, anthocyanidins, chalcones, dihydrochalcones, aurones, flavanols, dihydroflavanols, proanthocyanidins (flavan-3,4-diols), isoflavones and neoflavones. Flavonoids also includes the molecule hesperetin. A “metal chelate” of a flavonoid refers to a non-covalent association of metal ion, such as, Fe(III) with a compound.

As used herein, the term “flavonoids” encompasses, but is not limited to:

chrysin, 5,7-dihydroxy-2-phenyl-4H-1-benzopyran-4-one; 5,7-dihydroxyflavone; chrysidenon 1438;

luteolin, 2-(3,4-Dihydroxyphenyl)-5,7-dihydroxy-4H-1-benzopyran-4-one; 3′,4′,5,7-tetrahydroxyflavone; digitoflavone; cyanidenon 1470;

quercetin, 2-(3,4-Dihydroxyphenyl)-3,5,7-trihydroxy-4H-1-benzopyran-4-one; 3,3′,4′,5,7-pentahydroxyflavone; memtin; sophoretin; cyanidenolon 1522;

rutin, 3-[[6-O-(6-Deoxy-α-L-mannopyranosyl)-β-D-glucopyranosyl]oxy]-2-(3,4-dihydroxyphenyl)-5,7-dihydroxy-4H-1-benzopyran-4-one; rutoside; quercetin-3-rutinoside; 3,3′,4′,5,7-pentahydroxyflavone-3-rutinoside; melin; phytomelin; eldrin; ilixathin; sophorin; globularicitrin; paliuroside; osyritrin; osyritin; myrticolorin; violaquercitrin; Birutan; Rutabion; Rutozyd; Tanrutin; see The Merck Index (1989), Eleventh Edition, Merck & Co., Whitehouse Station, N.J., pp. 350, 441, 520, 738, 883, 1278, 1319 and references cited therein.

hesperidin, (S)-7-[[6-O-(6-Deoxy-α-L-mannopyranosyl)-β-D-glucopyranosyl]oxy]-2,3-dihydro-5-hydroxy-2-(3-hydroxy-4-methoxyphenyl)-4H-1-benzopyran-4-one; hesperetin 7-rhamnoglucoside; cirantin; hesperetin-7-rutinoside;

diosmin; by “diosmin” is meant 7-[[6-O-6-Deoxy-α-L-mannopyranosyl)-β-D-glucopyranosyl]oxy]-5-hydroxy-2-(3-hydroxy-4-methoxyphenyl)-4H-1-benzopyran-4-one; 3′,5,7-trihydroxy-4′-methoxyflavone-7-rutinoside; 5-hydroxy-2-(3-hydroxy-4-methoxyphenyl)-7-(O⁶-α-L-rhamnopyranosyl-β-D-glucopyranosyloxy)chromen-4-one; 5-hydroxy-2-(3-hydroxy-4-methoxyphenyl)-7-β-rutinosyloxy-4H-chromen-4-one; diosmetin 7-β-rutinoside; diosmine; barosmin; buchu resin; Daflon; Diosmil; Diovenor; Flebopex; Flebosmil; Flebosten; Flebotropin; Hemerven; Insuven; Tovene; Varinon; Ven-Detrex; Venex; Veno-V; or Venosmine. Derivatives of diosmin are described in, for example, U.S. Pat. Nos. 5,296,469; and 4,894,449. Diosmin is a flavonoid extract; isolation of diosmin from various plant sources is described in, for example, Oesterle et al. (1925) Helv. Chim. Acta 8:519; and Horowitz (1956) J. Org. Chem. 21:1184; Arthur et al. (1956) J. Chem. Soc. 632.

hesperetin; by “hesperetin” is meant the compound (S)-2,3-dihydro-5,7-dihydroxy-2-(3-hydroxy-4-methoxyphenyl)-4H-1benzopyran-4-one; 3′,5,7-trihydroxy-4′-methoxyflavanone; cyanidanon 4′-methyl ether 1626. See The Merck Index (1996), Twelfth Edition, Merck & Co., Whitehouse Station, N.J., p. 798 and references cited therein. Hesperetin can be prepared by extraction from the peel of citrus fruit or by synthesis. Shinoda et al. (1929) C. A. 23:2957; Seka et al. (1936) Monatsh. 69:284. The separation of isomers of hesperetin is described in Arthur et al. (1956) J. Chem. Soc. 632. The structure and configuration of hesperetin are described in Arakawa et al. (1960) Ann. 636:111.

By “daidzein” is meant 7-hydroxy-3-(4-hydroxyphenyl)-4H-1-benzopyran-4-one; or 4′,7-dihydroxyisoflavone. See The Merck Index (1996), Twelfth Edition, Merck & Co., Whitehouse Station, N.J., p. 475 and references cited therein. Isolation of daidzein from various plant products is described in Hosny et al. (1999) J. Nat. Prod. 62: 853-8; Walz (1931) Ann. 489:118; and Wong (1962) J. Sci. Food Agr. 13:304. Synthesis is described in Farkas et al. (1959) Ber. 92:819. Daidzein is an inactive analog of the tyrosine kinase inhibitor genistein. Sargeant et al. (1993) J. Biol. Chem. 268:18151. Daidzein is also a phytoestrogen, recently suggested to play a role in preventing special types of cancer. Sathyamoorthy et al. (1994) Cancer Res. 54:957; Zhou et al. (1999) J. Nutr. 129:1628-35; and Coward et al. (1993) J. Agric. Food Chem. 41: 1961. Daidzein also has anti-estrogen properties. Anderson et al. (1998) Baillieres Clin. Endocrinol. Metab. 12: 543-57. Daidzein also acts as an anti-oxidant, inhibiting lipid peroxidation. Arora et al. (1998) Arch. Biochem. Biophys. 356: 133-41; and Hodgson et al. (1999) Atherosclerosis 145: 167-72. Daidzein is also useful for treating Alzheimer's disease. U.S. Pat. Nos. 5,952,374; and 5,733,926. Daidzein also alters the concentration of cholesterol constituents in human blood. U.S. Pat. No. 5,855,892.

By “biochanin” or “biochanin A” is meant 5,7-dihydroxy-4′-methoxyiso-flavone; or olmelin. See The Merck Index (1996), Twelfth Edition, Merck & Co., Whitehouse Station, N.J., p. 744 and references cited therein. Biochanin A can be isolated from red clover. Pope et al. (1953) Chem. & Ind. (London) 1092; and Wong (1962) J. Sci. Food. Agr. 13:304. Its structure is described by Bose et al. (1950) J. Sci. Ind. Res. 9B: 25. Biochanin A also has some anti-cancer properties. Lyn-Cook et al. (1999) Cancer Lett. 142: 111-9; Hammons et al. (1999) Nutr. Cancer 33: 46-52; Yin et al. (1999) Thyroid 9: 369-76. Biochanin A also has anti-oxidant properties, including the ability to inhibit lipid peroxidation. Toda et al. (1999) Phytother. Res. 13: 163-5.

A “synergist” is defined as an agent or compound which when present results in a greater-than-additive increase, augmentation or enhancement of the effect of an agent or compound. In some cases, it may be difficult to determine which compound in a mixture is of primary importance and which only secondary. Thus, in a synergistic mixture of compounds, any of the active compounds within the mixture can be considered a synergist. A composition comprising “synergistic activity” or a “synergistic mixture” is a combination of compounds wherein the combined effect is greater than additive of the individual effects. For example, a synergistic mixture can comprise two or more tocopherols, or a tocopherol and a non-tocopherol such as hesperetin, quercetin, diosmin or lactoferrin. Synergism may be apparent only at some ranges or concentrations.

“Infarct” or “infarction” relates to a region of a tissue or organ subjected to ischemia and suffering the physiological sequelae of ischemia. Infarction results from a sudden insufficiency of arterial or venous blood supply due to, for example, emboli, thrombi, vascular torsion or pressure that produces a macroscopic area of necrosis. Infarction also relates to a region injured as a result of exposure to a hemorrhage.

By “increasing cerebral blood flow is meant the act of improving clinical outcome by inducing a statistically or physiologically significant increase in cerebral blood flow in a treated subject relative to an untreated subject as determined using techniques which are well known in the art, such as vascular imaging, for example.

By “reducing infarct size” is meant the act of improving clinical outcome by inducing a statistically or physiologically significant reduction in infarct size in a treated subject relative to an untreated subject as determined using techniques which are well known in the art, such as vascular imaging, for example.

By “non-naturally-occurring composition” is meant a composition which is not found in this form in nature. A non-naturally-occurring composition can be derived from a naturally-occurring composition, e.g., as non-limiting examples, via purification, isolation, concentration, chemical modification (e.g., addition or removal of a chemical group), and/or, in the case of mixtures, addition or removal of ingredients or compounds. Alternatively, a non-naturally-occurring composition can comprise or be derived from a non-naturally-occurring combination of naturally-occurring compositions. Thus, a non-naturally-occurring composition can comprise a mixture of purified, isolated, modified and/or concentrated naturally-occurring compositions, and/or can comprise a mixture of naturally-occurring compositions in forms, concentrations, ratios and/or levels of purity not found in nature.

“Agents” or “cytoprotective agents” are defined herein as compounds, mixtures, or formulations of compounds which are capable of preventing or treating a tissue ischemia, such as for example, cerebral ischemia, such as by reducing tissue or cell damage or symptoms thereof, associated with an ischemic condition and/or cell damage due to said ischemia condition. “Amelioration” means the prevention, reduction, palliation, or a counter-acting of the negative aspects of an ischemic condition or ischemic state. Amelioration does not require a complete recovery or complete prevention of an ischemic condition. A compound or agent may provide protective activity prior to, simultaneous with and/or after the tissue ischemia event has occurred.

As used herein, an agent is said to be “cytoprotective” or to have “cytoprotective property” or “cytoprotective activity” if administration of the agent reduces and/or ameliorates symptoms of a tissue ischemic condition and/or injury(ies) suffered by cells, tissues, organs and/or organisms that is induced secondary to the ischemic condition. Cytoprotective activity and injury can be quantified in assays which measure results of injury such as death and inhibition of metabolic activity; these can be measured, for example, using appropriate fluorescent dyes, measuring enzyme activity and/or measuring intact cellular membrane in effected tissues by staining with appropriate indicators. Cytoprotective agents include cytoprotective tocopherols, metabolites thereof and derivatives thereof, and flavonoids, and derivatives thereof.

A “nutritional composition” is a composition that comprises naturally occurring components, preferably found in the food supply, that can be sold over the counter, as supplements, functional foods or food ingredients ie, without a physician's prescription. A pharmaceutical composition is one that includes ethical pharmaceuticals and which requires a physician's prescription for administration. A nutritional composition will comprise a tocopherol to be administered in a range of about 1 to about 50 mg per kg body weight of said mammalian subject and/or a flavonoid in a range of about 1 to about 25 mg per kg body weight of said mammalian subject. A pharmaceutical composition will comprise a tocopherol to be administered in a range of about 1 to about 1000 mg per kg body weight of said mammalian subject and/or a flavonoid in a range of about 1 to about 1000 mg per kg body weight of said mammalian subject. A medicament as used herein encompasses nutritional compositions and pharmaceutical compositions.

By “amounts effective to reduce damage associated with a tissue ischemic conditions and/or symptoms due to tissue ischemia” is meant that the cytoprotective agent or agents (e.g., gamma-tocopherol, and/or beta-tocopherol, and/or delta tocopherol and/or a mixture thereof and/or derivatives and/or metabolites thereof and/or a flavonoid and/or derivatives thereof) is present in a final concentration sufficient for amelioration of injury(ies) associated with a tissue ischemic condition and/or symptoms due to a tissue ischemia. This amount includes, but is not limited to, a concentration which acts as a complete prophylaxis or treatment for a symptom of cellular or tissue damage, such as neuronal damage for cerebral ischemia. An “effective amount” is an amount sufficient to effect beneficial or desired results. An effective amount can be administered in one or more administrations. For purposes of this invention, an effective amount of a cytoprotective composition is an amount that is sufficient to ameliorate, stabilize, reverse, slow or delay the progression of injury(ies) in mammalian subjects i) at risk for an ischemic condition, or ii) associated with, due to and/or symptoms of an ischemic condition. Preferably, amelioration of injury(ies) due to a tissue ischemic condition can be quantified by an assay measuring, for example, reduction in cell death and/or enzyme inactivity and/or reduction of tissue edema and/or reduction in cognitive disorder and/or reducing infarct size, for example. In the case of injuries associated with a tissue ischemic condition, the size and/or severity of an infarct in the tissue, such as in the brain for cerebral ischemia, of the subject may be determined, for example, by various noninvasive radiological procedures and/or by various symptomatic and diagnostic procedures known to those of skill in the art, such as magnetic resonance imaging (MRI), computerized tomography (CT) scan, and the like. Injuries associated with a tissue ischemic condition also include edema and injuries that are associated with such edema. When used in relation to a tissue ischemic condition, the term “effective” when describing a dose size, frequency, or duration, or the concept of dose “effectiveness,” relates to a dosing which results in a reduction in the size and severity of an actual infarct, or to a probability that any such infarct, were it to occur, would be of reduced size and severity. Amelioration is preferably at least about 30%, preferably at least about 50%, more preferably at least about 70%, even more preferably at least about 80%, and even more preferably at least about 90% reduction in tissue damage, such as neuronal damage for cerebral ischemia.

A “mammalian subject” includes, but is not limited to, a human, a farm animal, a sport animal, and a pet.

By “treatment” or “treating” is meant any treatment of a disease or disorder, in a mammal, including: preventing or protecting against the disease or disorder, that is, causing, the clinical symptoms of the disease not to develop; inhibiting the disease, that is, arresting or suppressing the development of clinical symptoms; and/or relieving the disease, that is, causing the regression of clinical symptoms.

“As used herein, the term “comprising” and its cognates are used in their inclusive sense; that is, equivalent to the term “including” and its corresponding cognates.

“Hypoxia,” which is defined broadly as a condition under which a particular cell, organ or tissue receives an insufficient oxygen supply to allow normal function. More specifically, hypoxia can be measured as an average or mean environmental oxygen saturation level of less than 90%. Hypoxia is a direct result of ischemia since whenever blood supply is cut off, oxygen supply is also cut off. However, hypoxia can occur in other conditions, even if blood flow remains unaltered, including, but not limited to, carbon monoxide poisoning, drowning, suffocation and other forms of asphyxia.

The term “energetically-competent” refers to cells, cell lines or organisms which undergo aerobic respiration (oxidative metabolism). “Energetic incompetence” refers to the quality of cells incapable of undergoing aerobic respiration; such cells only perform anaerobic respiration (fermentation).

General Methods

General techniques for chemical manipulations are known in the art and are generally described in, for example, Haugland (1996) Handbook of Fluorescent Probes and Research Chemicals, Sixth Edition, Molecular Probes, Inc.; Carruthers (1986) Some Modern Methods of Organic Synthesis, Third Edition, Cambridge University Press; and Warren (1978) Designing Organic Syntheses, John Wiley & Sons, Ltd. Molecular biology techniques are generally described in, for example, Sambrook et al. (1989), Molecular Cloning: A Laboratory Manual, Second Edition; and Ausubel et al., eds. (1987) Current Protocols In Molecular Biology. Reagents useful in applying these techniques are widely known in the art and commercially available from a number of vendors.

Compositions

Provided herein are non-alpha tocopherol enriched tocopherol compositions comprising a tocopherol and that may further comprise a tocopherol metabolite, and/or tocopherol derivative and/or other tocopherols for use as cytoprotectants against damage, injury(ies) and/or symptoms associated with an ischemic condition. Also provided herein are gamma-tocopherol enriched tocopherol compositions comprising gamma-tocopherol that may further comprise gamma-tocopherol metabolites, and/or derivatives thereof, and/or other tocopherol metabolites, for use as cytoprotectants against damage, injury(ies) and/or symptoms associated with cerebral ischemia.

Also provided herein are gamma-tocopherol metabolite enriched compositions comprising gamma-tocopherol metabolite(s) that may further comprise gamma-tocopherol, and/or derivatives thereof, and/or other tocopherol metabolites, for use as cytoprotectants against damage, injury(ies) and/or symptoms associated with cerebral ischemia.

Provided herein are beta-tocopherol enriched tocopherol compositions comprising beta-tocopherol that may further comprise beta-tocopherol metabolites, and/or beta-tocopherol derivatives, and/or other tocopherols, eg, gamma- and delta-tocopherol for use as cytoprotectants against damage, injury(ies) and/or symptoms associated with tissue ischemia. Also provided herein are beta-tocopherol metabolite enriched compositions comprising beta-tocopherol metabolite(s) that may further comprise beta-tocopherol, and/or derivatives thereof, and/or other tocopherol metabolites, for use as cytoprotectants against damage, injury(ies) and/or symptoms associated with tissue ischemia.

Provided herein are delta-tocopherol enriched tocopherol compositions comprising delta-tocopherol that may further comprise delta-tocopherol metabolites, and/or delta-tocopherol derivatives, and/or other tocopherols, eg gamma- and beta-tocopherol, for use as cytoprotectants against damage, injury(ies) and/or symptoms associated with tissue ischemia. Also provided herein are delta-tocopherol metabolite enriched compositions comprising delta-tocopherol metabolite(s) that may further comprise delta-tocopherol, and/or derivatives thereof, and/or other tocopherol metabolites, for use as cytoprotectants against damage, injury(ies) and/or symptoms associated with tissue ischemia.

Provided herein are flavonoid enriched compositions and flavonoid derivative enriched compositions and compositions comprising gamma-tocopherol and a flavonoid for use as cytoprotectants against damage, injury(ies) and/or symptoms associated with cerebral ischemia.

These compounds are present in the compositions in amounts effective to ameliorate the injury(ies) and/or symptoms associated with a tissue ischemia, such as cerebral ischemia. Preferably gamma-tocopherol enriched compositions comprise at least 50% gamma-tocopherol, at least 55% gamma-tocopherol, at least 60% gamma-tocopherol, at least 65% gamma-tocopherol, at least 70% gamma-tocopherol, at least 75% gamma-tocopherol, at least 80% gamma-tocopherol, at least 85% gamma tocopherol, at least 90% gamma-tocopherol and at least 95% gamma-tocopherol. Gamma-tocopherol enriched tocopherol compositions may also comprise gamma-tocopherol derivative(s) and/or gamma tocopherol metabolite(s), and/or other tocopherol(s) and/or mixtures thereof. Gamma-tocopherol enriched tocopherol compositions comprises less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 5% alpha-tocopherol.

Preferably, gamma-tocopherol metabolite enriched compositions comprise at least 50% gamma-tocopherol metabolite, at least 55% gamma-tocopherol metabolite, at least 60% gamma-tocopherol metabolite, at least 65% gamma-tocopherol metabolite, at least 70% gamma-tocopherol metabolite, at least 75% gamma-tocopherol metabolite, at least 80% gamma-tocopherol metabolite, at least 85% gamma tocopherol metabolite, at least 90% gamma-tocopherol metabolite and at least 95% gamma-tocopherol metabolite. In preferred embodiments, gamma-tocopherol metabolite enriched compositions comprises less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 5% alpha-tocopherol. Gamma-tocopherol metabolite enriched compositions may also comprise gamma-tocopherol and/or a gamma tocopherol derivative(s), and/or other tocopherol(s) and/or mixtures thereof.

Preferably beta-tocopherol enriched compositions comprise at least 50% beta-tocopherol, at least 55% beta-tocopherol, at least 60% beta-tocopherol, at least 65% beta-tocopherol, at least 70% beta-tocopherol, at least 75% beta-tocopherol, at least 80% beta-tocopherol, at least 85% beta tocopherol, at least 90% beta-tocopherol and at least 95% beta-tocopherol. Beta-tocopherol enriched tocopherol compositions may also comprise beta-tocopherol derivative(s) and/or beta tocopherol metabolite(s), and/or other tocopherol(s) and/or mixtures thereof. Beta-tocopherol enriched tocopherol compositions comprises less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 5% alpha-tocopherol.

Preferably, beta-tocopherol metabolite enriched compositions comprise at least 50% beta-tocopherol metabolite, at least 55% beta-tocopherol metabolite, at least 60% beta-tocopherol metabolite, at least 65% beta-tocopherol metabolite, at least 70% beta-tocopherol metabolite, at least 75% beta-tocopherol metabolite, at least 80% beta-tocopherol metabolite, at least 85% beta tocopherol metabolite, at least 90% beta-tocopherol metabolite and at least 95% beta-tocopherol metabolite. In preferred embodiments, beta-tocopherol metabolite enriched compositions comprises less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 5% alpha-tocopherol. Beta-tocopherol metabolite enriched compositions may also comprise beta-tocopherol and/or a beta tocopherol derivative(s), and/or other tocopherol(s) and/or mixtures thereof.

Preferably delta-tocopherol enriched compositions comprise at least 50% delta-tocopherol, at least 55% delta-tocopherol, at least 60% delta-tocopherol, at least 65% delta-tocopherol, at least 70% delta-tocopherol, at least 75% delta-tocopherol, at least 80% delta-tocopherol, at least 85% delta tocopherol, at least 90% delta-tocopherol and at least 95% delta-tocopherol. Delta-tocopherol enriched tocopherol compositions may also comprise delta-tocopherol derivative(s) and/or delta tocopherol metabolite(s), and/or other tocopherol(s) and/or mixtures thereof. Delta-tocopherol enriched tocopherol compositions comprises less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 5% alpha-tocopherol.

Preferably, delta-tocopherol metabolite enriched compositions comprise at least 50% delta-tocopherol metabolite, at least 55% delta-tocopherol metabolite, at least 60% delta-tocopherol metabolite, at least 65% delta-tocopherol metabolite, at least 70% delta-tocopherol metabolite, at least 75% delta-tocopherol metabolite, at least 80% delta-tocopherol metabolite, at least 85% delta tocopherol metabolite, at least 90% delta-tocopherol metabolite and at least 95% delta-tocopherol metabolite. In preferred embodiments, delta-tocopherol metabolite enriched compositions comprises less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 5% alpha-tocopherol. Delta-tocopherol metabolite enriched compositions may also comprise delta-tocopherol and/or a delta tocopherol derivative(s), and/or other tocopherol(s) and/or mixtures thereof.

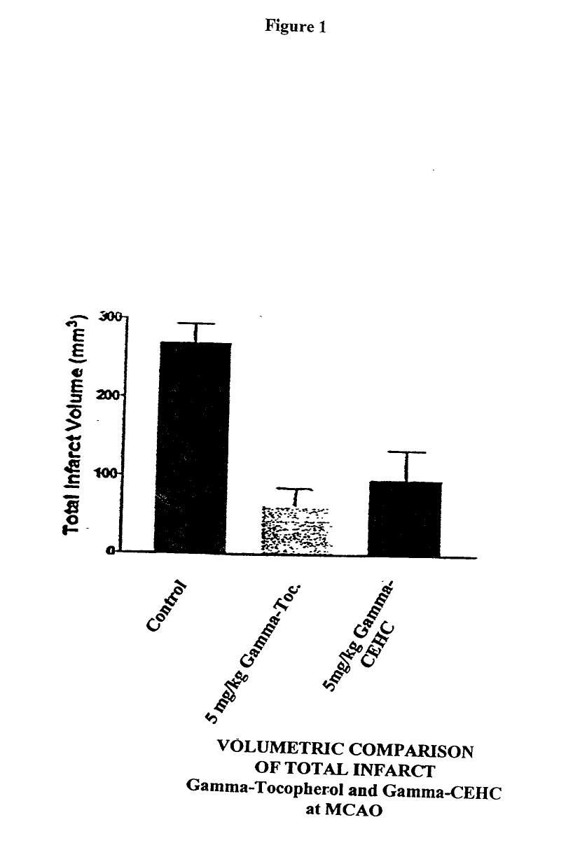

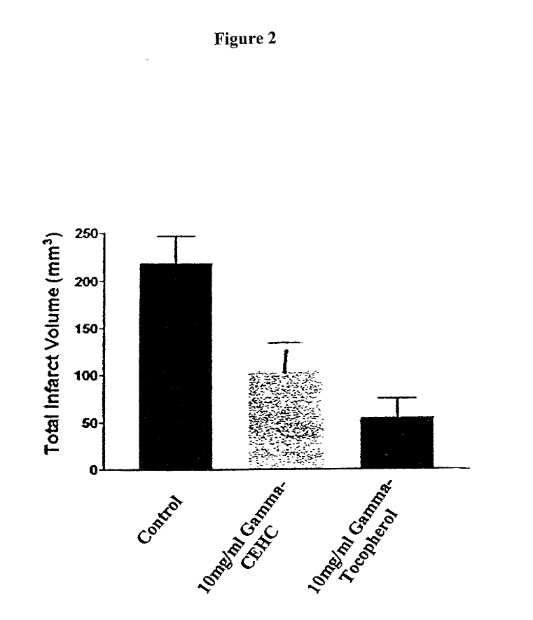

In illustrative examples disclosed herein, a gamma-tocopherol enriched composition (obtained from Sigma and comprising greater than 97% gamma-tocopherol) and a gamma-tocopherol metabolite enriched composition, gamma-CEHC (greater than 98% gamma-CEHC) are able to reduce total infarct size in an animal model of cerebral ischemia, the middle cerebral artery occlusion (MCAO model), when administered at the time of MCAO and when administered at reperfusion (see Example 2 and FIGS. 1 and 2, respectively). In additional experiments, total infarct size was measured for various tocopherol containing compositions at before, during MCAO and at reperfusion as described in Example 2. A gamma-tocopherol enriched composition comprising greater than 97% gamma-tocopherol and administered IV at 0.6 and 6 mg/kg at MCAO reduced infarct size by 81% and 82%, respectively, whereas an alpha-tocopherol containing composition comprising greater than 99% alpha-tocopherol administered at 6 mg/kg at MCAO reduced infarct by 42%. In additional experiments, a delta-tocopherol enriched composition comprising greater than 90% delta tocopherol when administered IV at 0.60 mg/kg was able to reduce infarct size by 52%. It was also shown that gamma-CEHC enriched composition decreased neuronal damage by 50-65% in drug ranges 0.1-5 mg/kg when administered IV at MCAO.

In further experiments in support of the present invention, test compounds were administered at reperfusion, as described in Example 2. Gamma-tocopherol (10 mg/kg) administration resulted in a reduction of infarct volume by 70%, while gamma-CEHC (10 mg/kg) administration resulted in a reduction of infarct volume by 52%.

In additional experiments carried out in support of the present invention, it was shown that when administered 3 hours pre-MCAO, a gamma-tocopherol enriched composition was able to reduce infarct size by 53% and 77%, respectively, when administered by oral gavage at 4 mg/kg and 10 mg/kg, respectively Similarly, a beta-tocopherol containing composition (obtained from Matreys, Inc. Pleasant Gap, Pa. and comprising greater than 90% beta-tocopherol) when administered oral gavage pre-MCAO at 10 mg/kg was reduced infarct size by 76%. A delta-tocopherol enriched composition administered by oral gavage pre-MCAO reduced infarct size by 48%, while alpha-tocopherol reduced infarct size by 43%.

Further experiments were conducted in which compound was administered intraperitoneally in the MCAO animal model described in Example 2. In an exemplary study, gamma-tocopherol (90% gamma-tocopherol, Sigma) administered at 10-20 mg/kg, either at MCAO or 3 hours post-reperfusion resulted in mean reduction of infarct volumes in the range of 20-57%.

In further experiments, tocopherols were tested in combination with flavonoid compounds, as described herein. In an exemplary experiment, when administered by oral gavage 3 hours pre-MCAO, 4 mg/kg gamma-tocopherol produced a 53% reduction in infarct volume, while 6 mg/kg quercetin+hesperetin resulted in 38% reduction of infarct volume. Combination of these compounds (4 mg/kg gamma-tocopherol and 6 mg/kg quercetin+hesperetin) resulted in 73% reduction in infarct volume.

In additional experiments carried out in support of the present invention, the flavonoid derivative, bromo-quercetin, was found to be effective at preventing neuronal cell death in a standard animal model of stroke, the rat middle cerebral artery occlusion (MCAO) described herein at low micromolar concentrations, as evidenced by reductions of infarct volumes of greater than 50%, when compound was administered intraventricularly (i.c.v.) at concentrations ranging from 0.0067 mg/kg to 0.67 mg/kg.

In further experiments in support of the present invention, 37-111 micromolar bromo-quercetin was also effective in preventing or reducing myocardial damage in the isolated heart cell (myocyte) preparation model of myocardial cell ischemia described in Example 4. Further, when given at a dose of 0.67 mg/kg, bromoquercetin was found to reduce infarct size in the animal model of myocardial infarction detailed in Example 5.

Compositions of the present invention were also shown to reduce ischemic damage associated with congestive heart failure (CHF) in experiments using the animal model of CHF detailed in Example 6. In this model, increased heart weight subsequent to permanent ligation of the left coronary artery is used as a measure of cardiac failure; reduction of heart weight after intervention (drug treatment) indicates improvement. In experiments carried out in support of the present invention, intervention with a gamma-tocopherol enriched composition (>65% gamma-tocopherol) resulted in reduction in heart weight with no decrease in left ventricular systolic pressure (LVSP). By way of comparison, captopril (1-[(2S)-3-mercapto-2-methylpropionyl]-L-proline), an ACE-I inhibitor used to treat CHF also reduced heart weight, but this treatment also resulted in reduced LVSP. Clinically, maintenance of LVSP levels is considered a desired outcome in CHF, since reduction in LVSP indicates reduced perfusion pressure.

Tocopherols are chemical entities which, in general, contain a 6-chromanol ring structure and a side chain at the 2-position. Prototypical tocopherols include alpha-, beta-, gamma- and delta-tocopherol. However, as is known in the art, tocopherols and their derivatives can vary by the number and position of alkyl groups, double bonds and other substituents and variations on the ring and side chain. An “alkyl” is a cyclic, branched or straight chain chemical group containing only carbon and hydrogen, such as methyl, butyl and octyl. Alkyl groups can be either unsubstituted or substituted with one or more substituents, e.g., halogen, alkoxy, acyloxy, amino, hydroxyl, mercapto, carboxy, or benzyl. Alkyl groups can be saturated or unsaturated at one or several positions. Typically alkyl groups will comprise 1 to 8 carbons, preferably 1 to 6, and more preferably 1 to 4 carbon atoms. Additional tocopherols can be constructed by conjugation to the ring structure or side chain of various other moieties, such as those containing oxygen, nitrogen, sulfur and/or phosphorus. Tocopherol derivatives can also be made, as known in the art, by modifying the length of the side chain from that found in prototypical tocopherols such as alpha-, beta-, delta- and gamma-tocopherol. Tocopherols can also vary in stereochemistry and saturation of bonds in the ring structure and side chain. Additional tocopherol derivatives, including prodrugs, can be made by conjugation of sugars or other moieties to the side chain or ring structure; these can serve any of a number of functions, including increasing solubility and increasing functional activity of the tocopherol. Thus, as is understood in the art, the invention encompasses the use of tocopherol derivatives in which substitutions, additions and other alterations have been made in the 6-chromanol ring and/or side chain, with the proviso that the derivatives maintain at least one functional activity of a tocopherol, such as antioxidant activity or ability to counteract sterility in animals. The tocopherols have the general formula:

Tocopherols:

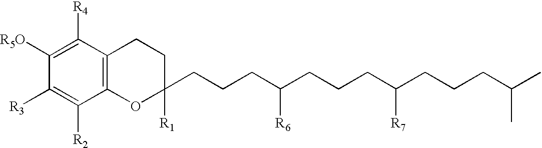

R1 = CH3 with S or R configuration
R6 = CH3 with S or R configuration
R7 = CH3 with S or R configuration
R5 = H or CH3 or acetate or succinate

| | R2 | R3 | R4 |
|---|---|---|---|
| Gamma | CH3 | CH3 | H |
| Beta | CH3 | H | CH3 |
| Delta | CH3 | H | H |

Gamma-tocopherol metabolites and derivatives are disclosed in Wechter et al., U.S. Pat. Nos. 6,150,402; 6,083,982 and 6,048,891. Other tocopherol metabolites or derivatives include tocotrienols, naturally occurring analogs of tocopherols and metabolites having the general formulas as shown below. Other metabolites are shown in FIG. 3.

Tocotrienols:

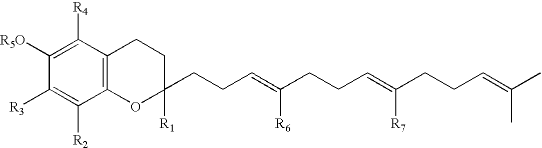

R1 = CH3 with S or R configuration
R6 = CH3 with S or R configuration
R7 = CH3 with S or R configuration
R5 = H or CH3 or acetate or succinate

| | R2 | R3 | R4 |
|---|---|---|---|
| Gamma | CH3 | CH3 | H |
| Beta | CH3 | H | CH3 |
| Delta | CH3 | H | H |

Natural compounds: Some naturally occurring chromene analogs of tocopherols are disclosed in WO00056348A1.

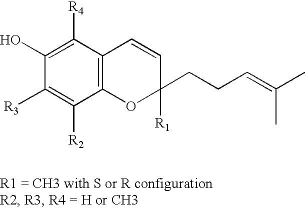

Examples of metabolites and derivatives include those of Formula 1, below (substituted chromans):

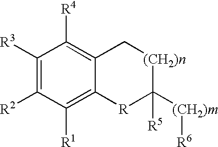

in which:

R is O, S, SO, SO₂, a secondary or tertiary amine group, a phosphate group, a phosphoester group, or an unsubstituted or substituted methylene group;

R¹ and R² independently are H, OH, alkyl, aryl, alkenyl, alkynyl, aromatic, ether, ester, unsubstituted or substituted amine, amide, halogen or unsubstituted or substituted sulfonyl, or jointly complete a 5- or 6-membered aliphatic or aromatic ring;

R³ and R⁴ independently are H, OH, alkyl, aryl, alkenyl, alkynyl, aromatic, ether, ester, unsubstituted or substituted amine, amide, halogen or unsubstituted or substituted sulfonyl, or jointly complete a 5- or 6-membered aliphatic, aromatic or heterocyclic ring;

R⁵ is H, OH, alkyl, aryl, alkenyl, alkynyl, aromatic, ester or unsubstituted or substituted amine;

R⁶ is COOH, COOR⁷, CONH₂, CONHR⁷, CONR⁷R⁸, NH₂, NHR⁷, NR⁷R⁸, or a carboxylate salt;

R⁷ and R⁸ independently are unsubstituted or substituted alkyl, aryl, alkaryl, aralkyl, alkenyl or alkynyl;

n is 0 to 3; and

m is 0 to 5. In preferred embodiments of Formula I, R is O. Also preferably, n=1, m=2, R⁶ is COOH, R³ is H or OH, R⁴ is H or CH₃. In a preferred embodiment, R¹, R² and R⁵ are CH³.

Exemplary compounds include: R is O, R¹, R², and R⁵ are CH₃, R³is OH, R⁴ is H or CH³, R⁶ is COOH, n=1 and m=2. (where R⁴ is H, this is gamma-CEHC).

Other exemplary compounds of Formula 1 includes those in which R is O, R¹, R², and R⁵ are CH₃, R³ is H, R⁴ is H or CH³, R⁶ is COOH, n=1 and m=2. (where R⁴ is H, this is 2,7,8-trimethyl-2-(β-carboxyethyl)-chroman).

In a preferred embodiment, R⁷ is a C₁₋₆ alkyl group, in particular CH₃. In another preferred embodiment, R³ is OH.

Specific examples of gamma-tocopherol metabolites include for example, gamma-CEHC: synonyms: 6-hydroxy-2,7,8-trimethylchroman-2-propanoic acid and 2,7,8-trimethyl-2-(β-carboxyethyl)-6-hydroxy chroman, and having formula:

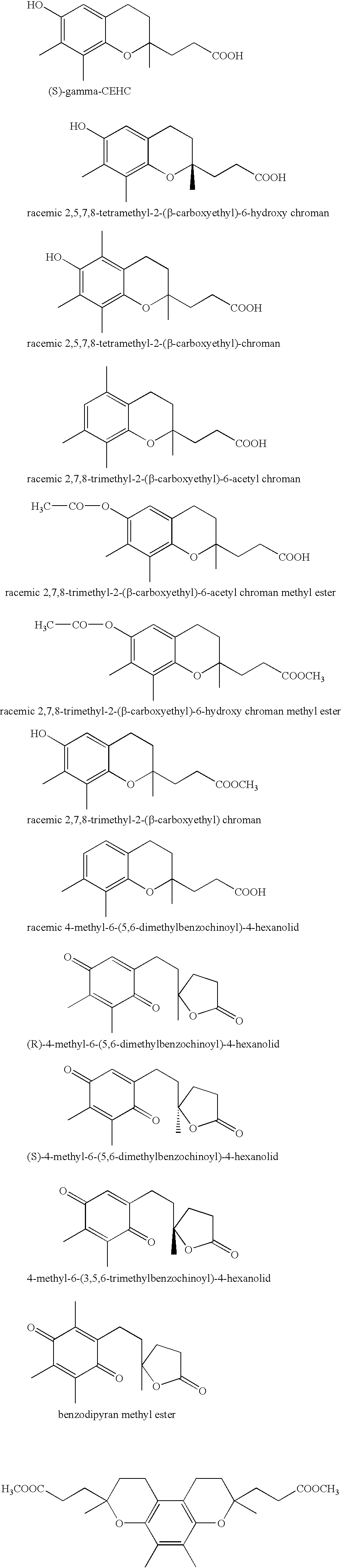

Examples of metabolites and derivatives include those of Formula II, below:

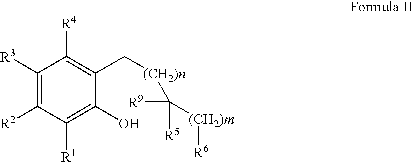

wherein

R¹ and R² independently are H, OH, alkyl, aryl, alkenyl, alkynyl, aromatic, ether, ester, unsubstituted or substituted amine, amide, halogen or unsubstituted or substituted sulfonyl, or jointly complete a 5- or 6-membered aliphatic or aromatic ring;

R³ and R⁴ independently are H, OH, alkyl, aryl, alkenyl, alkynyl, aromatic, ether, ester, unsubstituted or substituted amine, amide, halogen or unsubstituted or substituted sulfonyl, or jointly complete a 5- or 6-membered aliphatic, aromatic or heterocyclic ring;

R⁵ is H, OH, alkyl, aryl, alkenyl, alkynyl, aromatic, ester or unsubstituted or substituted amine;

R⁶ is COOH, COOR⁷, CONH₂, CONHR⁷, CONR⁷R⁸, NH₂, NHR⁷, NR⁷R⁸, or a carboxylate salt;

R⁷ and R⁸ independently are unsubstituted or substituted alkyl, aryl, alkaryl, aralkyl, alkenyl or alkynyl;

R⁹ is hydroxyl or unsubstituted or substituted alkoxyl;

n is 0to 3; and

m is 0 to 5.

In preferred embodiments, R¹, R² and R⁵ are CH₃ and preferably, R³ is OH and R⁴ preferably is H. Additionally, it is preferred that n=1 and referred that m=2.

In a further preferred embodiment, R⁶ is COOCH₂CH₃ and R⁹ is OH. In another preferred embodiment, R⁶ is COOH and R⁹ is CH₃CH₂O. Specific exemplary examples include

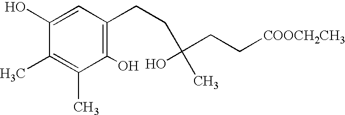

Examples of metabolites and derivatives include those of Formula III, below

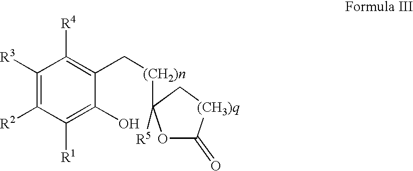

wherein

R¹ and R² independently are H, OH, alkyl, aryl, alkenyl, alkynyl, aromatic, ether, ester, unsubstituted or substituted amine, amide, halogen or unsubstituted or substituted sulfonyl, or jointly complete a 5- or 6-membered aliphatic or aromatic ring;

R³ and R⁴ independently are H, OH, alkyl, aryl, alkenyl, alkynyl, aromatic, ether, ester, unsubstituted or substituted amine, amide, halogen or unsubstituted or substituted sulfonyl, or jointly complete a 5- or 6-membered aliphatic, aromatic or heterocyclic ring;

R⁵ is H, OH, alkyl, aryl, alkenyl, alkynyl, aromatic, ester or unsubstituted or substituted amine;

n is 0 to 3; and

q is 0 to 4.

In preferred embodiments, n=1. Also in preferred embodiments, q=2.

Exemplary compounds of Formula III include the following:

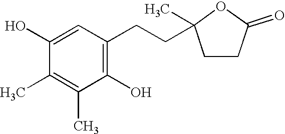

Examples of metabolites and derivatives include those of Formula IV, below

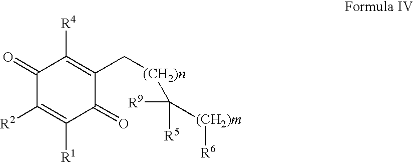

wherein

R¹ and R² independently are H, OH, alkyl, aryl, alkenyl, alkynyl, aromatic, ether, ester, unsubstituted or substituted amine, amide, halogen or unsubstituted or substituted sulfonyl, or jointly complete a 5- or 6-membered aliphatic or aromatic ring;

R⁴ is H, OH, alkyl, aryl, alkenyl, alkynyl, aromatic, ether, ester, unsubstituted or substituted amine, amide, halogen or unsubstituted or substituted sulfonyl;

R⁵ is H, OH, alkyl, aryl, alkenyl, alkynyl, aromatic, ester or unsubstituted or substituted amine;

R⁶ is COOH, COOR⁷, CONH₂, CONHR⁷, CONR⁷R⁸, NH₂, NHR⁷, NR⁷R⁸, or a carboxylate salt;

R⁷ and R⁸ independently are unsubstituted or substituted alkyl, aryl, alkaryl, aralkyl, alkenyl or alkynyl;

R⁹ is hydroxyl or unsubstituted or substituted alkoxyl;

n is 0 to 3; and

m is 0 to 5.

In preferred embodiments, n=1 and m=2.

Exemplary compounds according to Formula IV include:

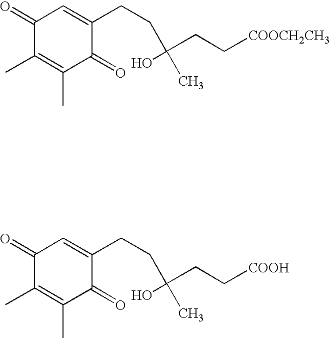

Examples of metabolites and derivatives include those of Formula V, below

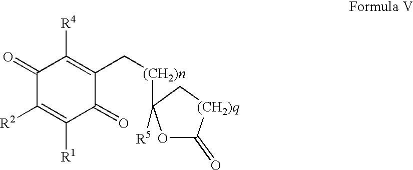

R¹ and R² independently are H, OH, alkyl, aryl, alkenyl, alkynyl, aromatic, ether, ester, unsubstituted or substituted amine, amide, halogen or unsubstituted or substituted sulfonyl, or jointly complete a 5- or 6-membered aliphatic or aromatic ring;

R⁴ is H, OH, alkyl, aryl, alkenyl, alkynyl, aromatic, ether, ester, unsubstituted or substituted amine, amide, halogen or unsubstituted or substituted sulfonyl;

R⁵ is H, OH, alkyl, aryl, alkenyl, alkynyl, aromatic, ester or unsubstituted or substituted amine;

n is 0 to 3; and

q is 0 to 4. In preferred embodiments, n=1 and q=2.

Exemplary compounds of Formula V include:

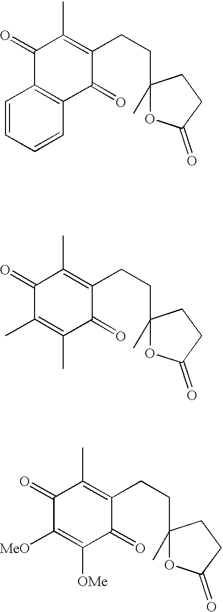

When compounds of Formula II-V are derived from the corresponding compounds and then oxidized, a preferred embodiment is R⁴=H, R⁵ is not CH₃.

Flavonoids include, but are not limited to, members of the following subclasses: chalcone, dihydrochalcone, flavanone, flavonol, dihydroflavonol, flavone, flavanol, isoflavone, neoflavone, aurone, anthocyanidin, proanthocyanidin (flavan-3,4-diol) and isoflavane. Flavanones contain an asymmetric carbon atom at the 2-position and flavanones include, but are not limited to, narigenin, naringin, eriodictyol, hesperetin and hesperidin. Dihydroflavonols include, but are not limited to, taxifolin (dihydroquercetin). Flavones include, but are not limited to, chrysin, diosmin, luetolin, apigenin, tangeritin and nobiletin. Flavonols include, but are not limited to, kampferol, quercetin and rutin. Flavanes include, but are not limited to, catechin and epi-gallocatechin-gallate. Isoflavones include, but are not limited to, biochanin, daidzein, glycitein and genistein. Also suitable in the present invention are derivatives of flavonoids. For example, derivatives of a flavonoid differ from the flavonoid in structure. These differences can be, as non-limiting examples, by addition, substitution or re-arrangement of hydroxyl, alkyl or other group. As a non-limiting example, a flavonoid derivative can have additional alkyl groups attached. In addition, flavonoid derivatives include compounds which have been conjugated to another chemical moiety, such as a sugar or other carbohydrate. Other suitable moieties contain oxygen, nitrogen, sulfur, and/or phosphorus. Derivatives of flavonoids can be produced, for example, to improve flavonoid solubility, reduce its odor or taste, or to ensure that the compound is free of toxicity. A flavonoid can also be conjugated to another moiety to form a prodrug. In a prodrug, a flavonoid is conjugated to a chemical moiety which, for example, aids in delivery of the flavonoid to the site of activity (e.g., a particular tissue within the body). This chemical moiety can be optionally cleaved off (e.g., enzymatically) at that site.

In certain embodiments, the flavonoid can be used in combination with a carbohydrate or carbohydrate derivative, including various carbohydrates and their phosphorylated derivatives including, but not limited to, ribulose, ribulose 1,5-bisphosphate, galactose, ribose, ADP-ribose, fructose, fructose-1,6-bisphosphate, pyruvate and β-hydroxybutyrate.

Additional compounds for use with the flavonoids in the compositions and methods of the invention include, but are not limited to, phosphoglycerates, where the phospho group at the 3 position is esterified with alkyl groups of from 1-2 carbon atoms and/or the 2-hydroxyl group is esterified with pyruvate. Flavonoids may also be mixed with tocopherols to form beneficial formulations, in accordance with the teachings of the present invention.

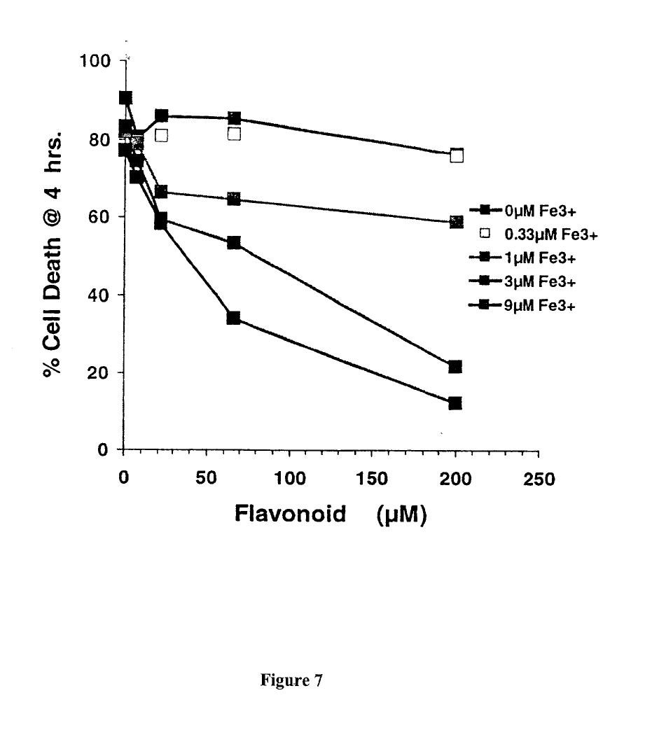

According to a preferred embodiment, flavonoids may be formulated to form metal chelates in solution. Such formulations have been shown, in accordance with the present invention, to provide particularly beneficial redox activity in cellular systems. FIG. 7 shows how a metal ion, Fe3+, can act synergistically with a flavonoid to effect cellular protection in an oxidative cell death assay, such as is described in Example 7 herein.

Hesperetin is exemplified herein as one of a class of compounds known as flavonoids, which are polyphenolic molecules based on a flavan nucleus, comprising 15 carbon atoms, arranged in three rings as C₆-C₃-C₆. Flavonoids are generally classified into subclasses by the state of oxidation and the substitution pattern at the C2-C3 unit. As used herein, the term “flavonoid” encompasses, but are not limited to, flavanones, flavonols, flavones, anthocyanidins, chalcones, dihydrochalcones, aurones, flavanols, dihydroflavanols, proanthocyanidins (flavan-3,4-diols), isoflavones and neoflavones. Flavanoids exemplified herein includes the molecules hesperetin, diosmin, daidzein and biochanin.

In experiments performed in support of the present invention, hesperetin was shown to act synergistically with gamma-tocopherol and delta-tocopherol.

Also suitable as for use in the present invention are derivatives of hesperetin that exhibit cytoprotective activity. The derivatives differ from hesperetin in structure. These differences can be, as non-limiting examples, by addition, substitution or re-arrangement of hydroxyl, alkyl or other group. As a non-limiting example, a hesperetin derivative can have additional alkyl groups attached. In addition, hesperetin derivatives include compounds which have been conjugated to another chemical moiety, such as a sugar or other carbohydrate. Other suitable moieties contain oxygen, nitrogen, sulfur, and/or phosphorus. Derivatives of hesperetin can be produced, for example, to improve its solubility, reduce its odor or taste, or to ensure that the compound is free of toxicity. Hesperetin can also be conjugated to another moiety to form a prodrug. In a prodrug, hesperetin is conjugated to a chemical moiety which, for example, aids in delivery of hesperetin to the site of activity (e.g., a particular tissue within the body). This chemical moiety can be optionally cleaved off (e.g., enzymatically) at that site. Hesperetin derivatives are described in, for example, Esaki et al. (1994) Biosci. Biotechnol. Biochem. 58:1479-85; Scambia et al. (1990) Anticancer Drugs 1:45-8; Bjeldanes et al. (1977) Science 197:577-8; Honohan et al. (1976) J. Agric. Food Chem. 24:906-11; and Brown et al. (1978) J. Agric. Food Chem. 26:1418-22.

While differing from hesperetin in structure, derivatives of hesperetin will retain at least one activity of hesperetin. These activities include anti-oxidant and anti-free radical activity. Saija et al. (1995) Free Radic. Biol Med. 19:481-6. Hesperetin is also an antilipolytic in rat adipocytes. Kuppusamy et al. (1993) Planta Med. 59:508-512. Hesperetin also has activity in controlling sebum production and treatment of side disorders. U.S. Pat. No. 5,587,176. Hesperetin may also act in inhibiting mammary tumorigenesis and proliferation of breast cancer cells. Guthrie et al. (1998) Adv. Exp. Med. Biol. 439:227-36; So et al. (1997) Cancer Lett. 112:127-33. Hesperetin also inhibits 7-ethoxycoumarin)-deethylase activity in rat liver microsomes. Moon et al. (1998) Xenobiotica 28:117-26. Hesperetin also reduces the susceptibility of membrane Ca²⁺-ATPase to thyroid hormone stimulation. Hesperetin also increases ocular blood flow. Liu et al. (1996) J. Ocul. Pharm. Ther. 12:95-101. Hesperetin also inhibits myeloperoxidase. 'T Hart et al. (1990) Chem. Biol. Interact. 73:323-35. Hesperetin also inhibits 3-hydroxy-3-methylglutaryl CoA reducatase. U.S. Pat. No. 5,763,414. Hesperetin derivatives retain at least one of these activities.

An alternative flavonoid suitable for the present invention is diosmin, or a derivative thereof. Diosmin is a flavonoid extract, 7-[[6-O-6-Deoxy-α-L-mannopyranosyl)-β-D-glucopyranosyl]oxy]-5-hydroxy-2-(3-hydroxy-4-methoxyphenyl)-4H-1-benzopyran-4-one. See The Merck Index (1996), Twelfth Edition, Merck & Co., Whitehouse Station, N.J., p. 558, and references cited therein.

Also suitable as for use in the present invention are derivatives of diosmin that exhibit cytoprotective activity. Derivatives of diosmin differ from diosmin in structure. These differences can be, as non-limiting examples, by addition, substitution or re-arrangement of hydroxyl, alkyl or other group. As a non-limiting example, a diosmin derivative can have at least one additional alkyl group added. In addition, diosmin derivatives include compounds which have been conjugated to another chemical moiety, such as a sugar or other carbohydrate. Other suitable moieties contain oxygen, nitrogen, sulfur and/or phosphorus. Diosmin can also be conjugated to another moiety to form a prodrug. Derivatives of diosmin include diosmin heptakis (hydrogensulfate) aluminum complex, and diosmin octakis (hydrogen sulfate) aluminum complex, as described in U.S. Pat. Nos. 5,296,469; and 4,894,449. Another derivative of diosmin is its aglycone form, diosmetin, 5,7-dihydroxy-2-(3-hydroxy-4-methoxypenyl)-4H-1-benzopyran-4-one. See The Merck Index (1996), Twelfth Edition, Merck & Co., Whitehouse Station, N.J., p. 558, and references cited therein. Derivatives of diosmin also include salts thereof. A synthetic diosmin derivative, LEW-10, is described in Azize et al. (1992) Chem. Phys. Lipids 63:169-77.

While differing from diosmin in structure, diosmin derivatives will retain at least one activity of diosmin. Diosmin is commonly administered to protect blood vessels and prevent and/or treat herpesvirus attacks. Diosmin also has free radical scavenger activity [Dumon et al. (1994) Ann. Biol. Clin. 52: 265-270]; is an antilipoperoxidant [Feneix-Clerc et al. (1994) Ann. Biol. Clin. 52:171-7]; inhibits 5′-nucleotidase [Kavutcu et al. (1999) Pharmazie 54:457-9]; attentuates lipopolysaccharide cytotoxicity in cell culture [Melzig et al. (1999) Pharmazie 54:29809]; probably affects cytochrome P450 activity [Teel et al. (1998) Cancer Lett. 133:135-141; and Ciolino et al. (1998) Cancer Res. 58:2754-60]. The combination of diosmin and hesperidin, known as Daflon 500 mg, has anti-inflammatory, anti-free radical, venotonic and vasculoprotective activities, in addition to attenuating reperfussion injury. Guillot et al. (1998) Pancreas 17:301-8; Amiel et al. (1998) Ann. Cardiol. Angeiol. 47:185-8; Nolte et al. (1997) Int. J. Microcirc. Clin. Exp. 17 (suppl. 1): 6-10; Delbarre et al. (1995) Int. J. Microcirc. Clin. Exp. 15 (suppl. 1): 27-33; Bouskela et al. (1995) Int. J. Microcirc. Clin. Exp. 15 (suppl. 1):22-6; and Friesenecker et al. (1995) Int. J. Microcirc. Clin. Exp. 15 (suppl. 1):17-21. The combination of diosmin and hesperidin is also useful for treating hemorrhoids. U.S. Pat. No. 5,858,371. A diosmin derivative retains at least one of these activities.

In preferred embodiments of the present invention, tocopherol enriched tocopherol compositions include for example:

gamma-tocopherol enriched composition comprising greater than 90% gamma-tocopherol and more preferably greater than 95% gamma-tocopherol;

gamma-CEHC enriched composition comprising greater than 90% gamma-CEHC and preferably greater than 95% gamma-CEHC;

gamma-tocopherol enriched composition comprising gamma-CEHC;

gamma-tocopherol enriched composition for intravenous injection comprising at least 50% gamma-tocopherol and delta-tocopherol;

gamma-tocopherol enriched composition for gavage administration comprising at least 50% gamma-tocopherol and beta-tocopherol;

beta-tocopherol compositions comprising greater than 90% beta-tocopherol and more preferably, greater than 95% beta-tocopherol; and

delta-tocopherol compositions comprising greater than 90% delta-tocopherol and more preferably greater than 95% delta-tocopherol.

Activity of a gamma-, beta-, or delta-tocopherol enriched tocopherol composition or a gamma-, beta, or delta-tocopherol metabolite enriched composition can be experimentally tested, for example, in an assay which measures ability to ameliorate injury(ies) or damage associate with a tissue ischemic condition. Such assays (which are detailed in Examples) include without limitation the use of hippocampal cell assay, animal cerebral infarct assay and animal assay for behavioral recovery after cerebral ischemia, and congestive heart failure model (CHF). Gamma-, beta-, or delta-tocopherol enriched tocopherol compositions and gamma-, beta-, or delta-tocopherol metabolite enriched compositions suitable for the present invention include those which are capable of ameliorating injury(ies) associated with a tissue ischemic condition, as indicated, for example, by a reduction in cell death, reduction in tissue edema associated with an ischemic condition, reduction in infarct size, reduction in cognitive disorder, such as in cerebral ischemia, as measured by the methods disclosed in the Examples. Reduction in cellular or tissue damage associated with a ischemic condition is quantified at least about 30%, preferably at least about 50%, more preferably at least about 70%, even more preferably at least about 80%, and even more preferably at least about 90% reduction. It is well known that gamma-tocopherol is metabolized in vivo to form, for example, gamma-CEHC and gamma-CEBC, among other metabolites. In humans, this metabolite is thought to be the result of sequential oxidation of its phytyl sidechain by enzymes that catalyze omega and beta oxidation. Therefore a further aspect of the present invention includes methods of treatment in which the patient is treated with agents effective to induce such oxidation, such as, for example, inducers of the enzyme cytochrome P450. Such inducers and methods are known in the art.

Additional compositions for use in the present invention include for example:

compositions comprising gamma-tocopherol, hesperetin and quercetin;

compositions comprising bromo-quercetin;

compositions comprising quercetin and hesperetin;

compositions comprising delta-tocopherol and hesperetin;

compositions comprising delta-tocopherol and quercetin;

compositions comprising delta-tocopherol, hesperetin and quercetin;

compositions comprising beta-tocopherol and hesperetin;

compositions comprising beta-tocopherol and quercetin; and

compositions comprising beta-tocopherol, hesperetin and quercetin;

In the present invention, the ranges of the components of a composition disclosed above, such as for example, gamma-tocopherol, hesperetin and quercetin or bromo-quercetin can be selected from any of the ranges given for the individual components. In one illustrative example, a composition comprising a tocopherol and a flavonoid has the ratio of tocopherol to flavonoid is 3:1. In other embodiments, the ratio of tocopherol to flavonoid wt/wt is 10:1; 5:1; 3:1; 2:1; 3:2; 4:3; 5:3; 5:4; 1:1; 4:5; 3:5; 2:3; 1:2; 1:3; 1:5; and 1:10.

In the present invention, a nutritional composition will comprise a tocopherol to be administered in a range of about 1 to about 50 mg per kg body weight of said mammalian subject and/or a flavonoid in a range of about 1 to about 25 mg per kg body weight of said mammalian subject. In additional embodiments, a nutritional composition will comprise a tocopherol to be administered at a lower limit of at least about 1.00, 1.50, 2.00, 2.50, 5.00, 7.50, 10.00, 12.50, 15.00, 17.50, 20.00, 22.25, and 25.00 mg per kg body weight of said mammalian subject and at an upper limit of not greater than about 27.25, 30.00, 32.50, 35.00, 37.25, 40.00, 42.25, 45.00, 47.25, and 50.00 mg per kg body weight of said mammalian subject, with the lower limit and upper limit to be selected independently. In additional embodiments, a nutritional composition will comprise a flavonoid to be administered at a lower limit of at least about 1.00, 1.50, 2.00, 2.50, 5.00, 7.50, and 10.00 mg per kg body weight of said mammalian subject and at an upper limit of not greater than about 12.50, 15.00, 17.50, 20.00, 22.50, and 25.00 mg per kg body weight of said mammalian subject, with the lower limit and upper limit to be selected independently. A pharmaceutical composition will comprise a tocopherol to be administered in a range of about 1 to about 1000 mg per kg body weight of said mammalian subject and/or a flavonoid in a range of about 1 to about 1000 mg per kg body weight of said mammalian subject. In additional embodiments, a pharmaceutical composition will comprise a tocopherol and/or a flavonoid to be administered at a lower limit of at least about 1, 10, 20, 30, 40, 50, 60, 70, 80, 90, 100, 200, 300, 400, and 500 mg per kg body weight of said mammalian subject and at an upper limit of not greater than about 60, 70, 100, 200, 300, 400, 500, 600, 700, 800, 900, and 1000 mg per kg body weight of said mammalian subject, with the lower limit and upper limit to be selected independently. In additional embodiments, a pharmaceutical composition will comprise a tocopherol to be administered in a range of about 10 to about 100 mg per kg body weight of said mammalian subject and/or a flavonoid in a range of about 10 to about 100 mg per kg body weight of said mammalian subject.

Methods of Using Compounds of the Invention

The compositions of the present invention are administered to a subject in amounts to reduce tissue cell damage. The subject may be experiencing a tissue ischemic condition, experiencing symptoms associated with or due to a tissue ischemic condition or be at risk for a tissue ischemic condition.

In one aspect, methods of the present invention relate to preventing neuronal damage in a mammalian subject at risk of developing injury due to a cerebral ischemic condition, e.g. for example, by an infarct in the brain. The methods of reducing neuronal damage relate to minimizing the extent and/or severity of injury in the brain associated with or due to a cerebral ischemic condition by ameliorating or reducing the injury that would otherwise occur. The methods encompass administering a non-alpha tocopherol, such as, a gamma-tocopherol enriched tocopherol composition and/or a beta-tocopherol enriched tocopherol composition and/or a delta-tocopherol enriched tocopherol composition and/or a gamma-, beta-, or delta-tocopherol metabolite enriched composition, and/or a flavonoid enriched and/or a flavonoid derivative enriched composition to a subject. The amount administered and the duration of the treatment are effective to minimize the size and/or severity of the neuronal damage in the mammalian subject as measured by for example, reduction in neuronal cell death and/or reduction in cerebral edema associated with a cerebral ischemic condition and/or reduction in cognitive disorder and/or reduction in infarct size. Thus, it is anticipated that as a result of such treatment the size and/or severity of any neuronal damage that develops is minimized.

The present invention provides prophylactic treatments for neuronal damage including cell death and/or presence of tissue edema and/or cognitive dysfunction and/or cerebral infarcts which may be due to ischemic, hypoxic/anoxic, or hemorrhagic events. Gamma-, beta-, or delta-tocopherol enriched tocopherol compositions and/or a gamma-, beta-, or delta-tocopherol metabolite enriched compositions and/or flavonoid enriched and/or flavonoid derivative enriched compositions of the present invention are administered to a subject at risk of experiencing neuronal damage associated or due to a cerebral ischemic condition, and ameliorates the severity of the damage, should it occur. The method is intended for a subject at risk of neuronal damage that is associated with, or results from, an acute or chronic medical condition. Such conditions might arise as a result of medical or surgical treatment planned for the subject (e.g., angioplasty) or as a result of an emergent medical condition such as a stroke or severe blood loss. Other conditions which place a subject at risk for neuronal damage associated with a cerebral ischemic condition include a genetic predisposition to stroke or a condition that is understood to increase the probability of incurring a cerebral infarct such as atherosclerosis, previous stroke or transient ischemic attacks, diabetes mellitus, hypertension, hypercholesterolemia, a history of smoking and may also include schizophrenia, epilepsy, neurodegenerative disorders, Alzheimer's disease and Huntington's disease. Diagnostic and/or pathological characterization of stroke victims has identified numerous additional medical conditions producing stroke that are widely known to practitioners of internal and neurological medicine.

Additional medical conditions that place a subject at risk for neuronal damage associated with or due to a cerebral ischemic condition include, but are not limited to, thrombosis; vasculitis (including collagen vascular disease (e.g., temporal (giant cell) arteritis, polyarteritis nodosa, Wegener's granulatosis, Takayasu's arteritis, syphilis), meningitis (e.g., tuberculosis, fungi, syphilis, bacteria, herpes zoster), arterial dissection (e.g., carotid, vertebral, intracranial arteries at the base of the brain), hematologic disorders (e.g., polycythemia, thrombocytosis, thrombotic thrombocytopenic purpura, disseminated intravascular coagulation, dysproteinemias, hemogiobinopathies (sickle cell disease)) and that caused by cocaine, amphetamines, moyamoya disease, fibromuscular dysplasia and Binswanger's disease); embolism (including cardiac sources (e.g., dysrhythmia, coronary heart disease, rheumatic heart disease, etc.), coronary artery bypass graft (CABG), atherothrombotic arterial sources (e.g., bifurcation of common carotid artery, carotid siphon, distal vertebral artery, aortic arch) and unknown sources (e.g., may be associated with a hypercoagulable state secondary to systemic disease, carcinoma (especially pancreatic), eclampsia, oral contraceptives, lupus, factor C or S deficiency, Factor V mutation such as Factor V Leiden, etc.)); vasoconstriction (including vasospasm (e.g., cerebral vasospasm following subarachnoid hemorrhage) and reversible cerebral vasoconstriction (e.g., idiopathic, migraine, eclampsia, trauma); and venous conditions (including dehydration, pericranial infection, postpartum and postoperative states and systemic cancer).

Medical conditions that place a subject at risk of neuronal damage associated with or due to a cerebral ischemic condition due to intracranial hemorrhage include, but are not limited to, spontaneous intracerebral hemorrhage (e.g., hypertensive, amyloid angiopathy); ruptured aneurysm (e.g., saccular, mycotic); ruptured arteriovenous malformation; drug use (e.g., cocaine, amphetamines); trauma; bleeding with brain tumors; systemic bleeding disorders (including anticoagulation therapy) and hemorrhagic infarction.

In another aspect, methods of the present invention relate to preventing myocardial damage in a mammalian subject at risk of developing injury due to a cardiovascular ischemic condition, e.g. for example, by a myocardial infarction or CHF. The methods of reducing myocardial damage relate to minimizing the extent and/or severity of injury in the heart associated with or due to a myocardial ischemic condition by ameliorating or reducing the injury that would otherwise occur. The methods encompass administering a beta-tocopherol enriched tocopherol composition and/or a delta-tocopherol enriched tocopherol composition and/or a beta-, or delta-tocopherol metabolite enriched composition and/or a flavonoid enriched and/or a flavonoid derivative enriched composition to a subject. The amount administered and the duration of the treatment are effective to minimize the size and/or severity of the myocardial damage in the mammalian subject as measured by for example, reduction in myocardial cell death and/or reduction in myocardial edema associated with a myocardial ischemic condition and/or reduction in myocardial infarct size. Thus, it is anticipated that as a result of such treatment the size and/or severity of any myocardial damage that develops is minimized.

The present invention provides prophylactic treatments for myocardial damage including cell death and/or presence of myocardial edema and/or myocardial infarcts which may be due to ischemic, hypoxic/anoxic, or hemorrhagic events. Beta- or delta-tocopherol enriched tocopherol compositions and/or a beta- or delta-tocopherol metabolite enriched compositions and/or flavonoid enriched and/or a flavonoid derivative enriched compositions of the present invention are administered to a subject at risk of experiencing myocardial damage associated or due to a myocardial ischemic condition, and ameliorates the severity of the damage, should it occur. The method is intended for a subject at risk of myocardial damage that is associated with, or results from, an acute or chronic medical condition. Such conditions might arise as a result of medical or surgical treatment planned for the subject (e.g., angioplasty) or as a result of an emergent medical condition such as a myocardial infarction or severe blood loss. Other conditions which place a subject at risk for myocardial damage associated with a myocardial ischemic condition include a genetic predisposition to myocardial infarction or a condition that is understood to increase the probability of incurring a myocardial infarct such as atherosclerosis, CHF, previous myocardial infarction or transient ischemic attacks, diabetes mellitus, hypertension, hypercholesterolemia, and a history of smoking.

In another aspect, methods of the present invention relate to preventing intestinal damage in a mammalian subject at risk of developing injury due to a intestinal ischemic condition. The methods of reducing intestinal damage relate to minimizing the extent and/or severity of injury in the intestinal tissue associated with or due to an intestinal ischemic condition by ameliorating or reducing the injury that would otherwise occur. The methods encompass administering a gamma-tocopherol enriched tocopherol composition and/or a beta-tocopherol enriched tocopherol composition and/or a delta-tocopherol enriched tocopherol composition and/or a gamma-, beta-, or delta-tocopherol metabolite or derivative enriched composition and/or a flavonoid enriched and/or a flavonoid derivative enriched composition to a subject. The amount administered and the duration of the treatment are effective to minimize the size and/or severity of the intestinal damage in the mammalian subject as measured by for example, reduction in intestinal cell death and/or reduction in intestinal edema associated with an intestinal ischemic condition and/or reduction in intestinal infarct size. Thus, it is anticipated that as a result of such treatment the size and/or severity of any intestinal damage that develops is minimized.

The present invention provides prophylactic treatments for intestinal damage including cell death and/or presence of intestinal edema and/or intestinal infarcts which may be due to ischemic, hypoxic/anoxic, or hemorrhagic events. Gamma-, beta-, or delta-tocopherol enriched tocopherol compositions and/or a gamma-, beta-, or delta-tocopherol metabolite or derivative enriched compositions and/or a flavonoid enriched and/or a flavonoid derivative enriched compositions of the present invention are administered to a subject at risk of experiencing intestinal damage associated or due to an intestinal ischemic condition, and ameliorates the severity of the damage, should it occur. The method is intended for a subject at risk of intestinal damage that is associated with, or results from, an acute or chronic medical condition. Such conditions might arise as a result of medical or surgical treatment planned for the subject or as a result of an emergent medical condition such as severe blood loss. Other conditions which place a subject at risk for intestinal damage associated with an intestinal ischemic condition include conditions which directly or indirectly cause intestinal ischemia (e.g., premature birth; birth asphyxia; congenital heart disease; cardiac disease; polycythemia; hypoxia; exchange transfusions; low-flow states; atherosclerosis, embolisms or arterial spasms; ischemia resulting from vessel occlusions in other segments of the bowel; ischemic colitis; and intestinal torsion such as occurs in infants and particularly in animals) and conditions which are directly or indirectly caused by intestinal ischemia (e.g., necrotizing enterocolitis, shock, sepsis, and intestinal angina).

In another aspect, methods of the present invention relate to preventing spinal cord damage in a mammalian subject at risk of developing injury due to a spinal cord ischemic condition. The methods of reducing spinal cord damage relate to minimizing the extent and/or severity of injury in the spinal cord tissue associated with or due to an spinal cord ischemic condition by ameliorating or reducing the injury that would otherwise occur. The methods encompass administering a gamma-tocopherol enriched tocopherol composition and/or a beta-tocopherol enriched tocopherol composition and/or a delta-tocopherol enriched tocopherol composition and/or a gamma-, beta-, or delta-tocopherol metabolite or derivative enriched composition and/or a flavonoid enriched and/or a flavonoid derivative enriched composition to a subject. The amount administered and the duration of the treatment are effective to minimize the size and/or severity of the spinal cord damage in the mammalian subject as measured by for example, reduction in spinal cord cell death and/or reduction in spinal cord edema associated with a spinal cord ischemic condition. Thus, it is anticipated that as a result of such treatment the size and/or severity of any spinal cord damage that develops is minimized.

The present invention provides prophylactic treatments for spinal cord damage including cell death and/or presence of spinal cord edema which may be due to ischemic, hypoxic/anoxic, or hemorrhagic events. Gamma-, beta-, or delta-tocopherol enriched tocopherol compositions and/or a gamma-, beta-, or delta-tocopherol metabolite enriched compositions and/or a flavonoid enriched and/or a flavonoid derivative enriched composition of the present invention are administered to a subject at risk of experiencing spinal cord damage associated or due to a spinal cord ischemic condition, and ameliorates the severity of the damage, should it occur. The method is intended for a subject at risk of spinal cord damage that is associated with, or results from, an acute or chronic medical condition. Such conditions might arise as a result of medical or surgical treatment planned for the subject or as a result of an emergent medical condition such as severe blood loss. Other conditions which place a subject at risk for spinal cord damage associated with a spinal cord ischemic condition include trauma and surgery.

The present invention provides prophylactic treatments for ocular damage including age-related macular degeneration (AMD) which may involve retinal cell death and/or presence of ocular edema which may be due to ischemic, hypoxic/anoxic, or hemorrhagic events. Gamma-, beta-, or delta-tocopherol enriched tocopherol compositions and/or a gamma-, beta-, or delta-tocopherol metabolite enriched compositions and/or a flavonoid enriched and/or a flavonoid derivative enriched composition of the present invention are administered to a subject at risk of experiencing AMD associated or due to an ischemic condition, and ameliorates the severity of the damage, should it occur.

A nonhuman mammal is identified as a subject if the animal is to be subjected tissue infarction, or is to undergo surgical or invasive procedures comparable to those identified in the preceding paragraph for human subjects.

In the present invention, gamma-, beta-, or delta-tocopherol enriched tocopherol composition(s) and/or gamma-, beta-, or delta-tocopherol metabolite enriched composition(s) and/or flavonoid enriched and/or a flavonoid derivative enriched compositions are administered to a mammalian subject. In the case where an individual has been diagnosed as having suffered an ischemic event, such as a stroke or a result of suffocation, the administration of a composition of the present invention should begin as soon as possible after the ischemic event occurred, preferably within a few days (e.g., within 1, 2 or 3 days), or more preferably within a few hours (e.g., less than about 12, 8, 6, 4, or 2 hours), of the event. In the case where an individual is at risk of developing an infarct as a result of impending surgical or similar intervention or is otherwise at high risk of infarction, administration of the compositions of the present invention preferably begins as soon as the decision planning the intervention is made or the risk identified. In either case, the duration of the treatment is a few days, several days, or a few weeks, circumscribed by the time frame in which ischemic injury is understood, or expected, to occur.

The size of the dose of the gamma-, beta-, or delta-tocopherol enriched tocopherol composition and/or gamma-, beta-, or delta tocopherol metabolite enriched composition and/or gamma-, beta-, or delta-tocopherol derivative composition and/or a flavonoid enriched and/or a flavonoid derivative enriched composition to be administered depends on the route of administration and the medical condition of the subject, as well as, other individual parameters of the subject such as age, size and weight. Administration of the composition to an individual having suffered an ischemic event represents the most acute situation of those considered in this invention, since the ischemic event has already occurred and the need for immediate minimization of injury from infarction is extreme. This may necessitate a particular size of dose appropriate for the circumstances. A human subject or nonhuman animal scheduled for imminent surgery or medical intervention is in a somewhat less acute medical state. As a result the size of each dose may be different, and may be open to somewhat greater variation and still be within the practice of the invention. In general, the size of each dose for administration are described herein. Dose sizes for nonhuman mammals generally fall in the same range on a mg/kg basis.

For the reasons just summarized, the frequency of dosing may also vary. A more acute medical status may suggest a different dosing regime than one that is somewhat less acute. In general, the invention may be practiced by administering doses at a frequency ranging from about once or twice per week to about once daily.

Likewise for the reasons given above, the duration of dosing is subject to variability. For example, a stroke victim, being in an extremely acute medical condition, requires the effect of the method of the invention to be realized as early as possible. This clearly dictates a course of dosing that emphasizes a short duration. A subject facing scheduled surgery or medical intervention, or being otherwise at high risk for a stroke, having a less acute medical status, may be subjected to dosing for a different, generally longer, duration. In general, a victim of stroke may be administered the oral antigen for a duration ranging from about 3 days to about 10 days. On the other hand, a subject who is about to undergo medical or surgical treatment which enhances the risk of a cerebral ischemic condition in the brain may undergo dosing for a duration ranging from about 5 days to about 14 days. A subject who is at risk for developing a cerebral infarct associated with a chronic medical condition such as a genetic predisposition to stroke, diabetes mellitus, hypertension, hypercholesterolemia, and a history of smoking may be treated for a duration of at least 5 days.

The methods of the invention require the administration of gamma-, beta-, or delta-tocopherol enriched tocopherol compositions and/or gamma-, beta-, or delta-tocopherol metabolite enriched compositions and/or a derivative(s), thereof, or mixtures thereof, and/or a flavonoid enriched and/or a flavonoid derivative enriched composition in an effective amount. With regard to a cerebral ischemic condition, an effective amount is one sufficient to reduce neuronal damage resulting from the cerebral ischemic condition. A reduction of neuronal damage is any prevention of injury to the brain which otherwise would have occurred in a subject experiencing a cerebral ischemic event absent the treatment of the invention. Several physiological parameters may be used to assess reduction of brain injury, including, but not limited to, a smaller infarct size, improved cerebral regional blood flow and decreased intracranial pressure, for example, as compared to pretreatment patient parameters, untreated cerebral ischemic patients or cerebral ischemic patients receiving a control alone. The size of an infarct in a human patient having suffered a stroke may be determined, for example, by various noninvasive radiological procedures known to those of skill in the field of medicine, especially to those of skill in radiology and neurology. Examples of methods available in the field include, but are not limited to, computerized tomography (CT) scanning, magnetic resonance imaging (MRI), ultrasonic imaging, and targeted radiotracer imaging. The size of an infarct in a nonhuman mammal having been subjected to procedures which have produced a cerebral infarct, may be determined by similar noninvasive procedures as those available for use with human patients. In addition, in cases where a nonhuman mammal dies in the course of an experiment, the size of the infarct may be established by direct observation, by post mortem anatomical and histological examination. Such procedures are well known to those of skill in the fields of veterinary medicine, pathology, physiology, anatomy, and related fields and an example of such a procedure is exemplified herein.

The severity of an infarct in a human patient having suffered a stroke may be determined, for example, by various symptomatic and diagnostic procedures known to those of skill in the fields of medicine, especially to those of skill in neurology, hematology, and physical medicine, in addition to assessing the results of radiological and anatomical diagnosis that were discussed in the preceding paragraph. Kinetic, sensory, and cognitive behavior is affected in stroke patients. Medical diagnosis routinely includes such assessments in analysis of the status of stroke victims. For example, the effectiveness of the various doses of the claimed compositions may be assessed using standard measurements known in the art including, but not limited to, the Barthel Index, Modified Rankin Score, NIH Stroke Scale total, NIH Stroke Scale motor item, the number of days to discharge from the hospital, mortality and other neuropsychological battery scores.

In addition, stroke patients may be diagnosed by the methods of hematology. These may be used to assess the populations and cellular characteristics of immune cells in the circulation, as well as various enzymatic activities or cellular components from brain tissue. These activities or components are generally found in the blood of stroke victims but are typically absent or present at only low levels in subjects that have not suffered a stroke. Similar procedures may be applied to nonhuman mammals who have suffered ischemic injury as a result of medical or surgical procedures. The amounts or values of the various results obtained in these diagnostic tests may be evaluated with respect to values known in the various fields to represent normal or pathological states. As a result of evaluating the group of diagnostic results obtained as outlined above, the severity of the infarction may be assessed by workers of skill in the medical fields. The dose size, frequency, and the duration of treatment by the method of the present invention may be adjusted accordingly based on the severity of the infarction and the general medical condition of the patient.

The compositions, as described above, can be prepared as a medicinal preparation (such as an aqueous solution for injection) or in various other media, such as foods for humans or animals, including medical foods and dietary supplements. A “medical food” is a product that is intended for the specific dietary management of a disease or condition for which distinctive nutritional requirements exist. By way of example, but not limitation, medical foods may include vitamin and mineral formulations fed through a feeding tube or cancer or bum victims (referred to as enteral administration or gavage administration). A “dietary supplement” shall mean a product that is intended to supplement the human diet and is typically provided in the form of a pill, capsule, tablet or like formulation. By way of example, but not limitation, a dietary supplement may include one or more of the following ingredients: vitamins, minerals, herbs, botanicals, amino acids, dietary substances intended to supplement the diet by increasing total dietary intake, and concentrates, metabolites, constituents, extracts or combinations of any of the foregoing. Dietary supplements may also be incorporated into food stuffs, such as, functional foods designed to promote cerebral health or to prevent cerebral ischemia. If administered as a medicinal preparation, the composition can be administered, either as a prophylaxis or treatment, to a patient in any of a number of methods. The cytoprotective compositions may be administered alone or in combination with other pharmaceutical agents and can be combined with a physiologically acceptable carrier thereof. The effective amount and method of administration of the particular cytoprotective formulation can vary based on the individual subject, the stage of disease, and other factors evident to one skilled in the art. During the course of the treatment, the concentration of the subject compositions may be monitored to insure that the desired level is maintained.

Generally, the route(s) of administration useful in a particular application are apparent to one of skill in the art. Routes of administration include, but are not limited to, oral, topical, dermal, transdermal, transmucosal, epidermal, parenteral, gastrointestinal.

For in vitro or ex vivo administration, the compounds may be provided in the medium of the cells and/or organ, as a single bolus, by repetitive addition, by continual infusion, or the like.

For administration, the invention includes subject compositions suitable for oral administration including, but not limited to, pharmaceutically acceptable tablets, capsules, powders, solutions, dispersions, or liquids. For rectal administration, the subject compositions may be provided as suppositories, as solutions for enemas, or other convenient application. Otherwise, the subject compositions may be administered intravascularly, arterially or venous, subcutaneously, intraperitoneally, intraorganally, intramuscularly, or the like.

For administration, the formulations may conveniently be presented in unit dosage form and may be prepared by any methods well known in the art of pharmacy. Such methods include the step of bringing into association the active ingredients with the carrier which constitutes one or more accessory ingredients. In general, the formulations are prepared by uniformly and intimately bringing into association the active ingredients with liquid carriers or finely divided solid carriers or both, and then if necessary shaping the product.

For oral administration, suitable subject compositions include, but not limited to, pharmaceutically acceptable tablets, capsules, powders, solutions, dispersions, or liquids. Also, the subject compositions may be compounded with other physiologically acceptable materials which can be ingested including, but not limited to, foods, including, but not limited to, food bars, beverages, powders, cereals, cooked foods, food additives and candies.

When the composition is incorporated into various media such as foods, it may simply be orally ingested. The food can be a dietary supplement (such as a snack or wellness dietary supplement) or, especially for animals, comprise the nutritional bulk (e.g., when incorporated into the primary animal feed).

The amount of the composition ingested, consumed or otherwise administered will depend on the desired final concentration. Typically, the amount of a single administration of a composition of the invention can be about 0.1 to about 1000 mg per kg body weight, or about 0.5 to about 10,000 mg per day. Any of these doses can be further subdivided into separate administrations, and multiple dosages can be given to any individual patient. A typical dosage for vitamin E administration is 100-600 mg/kg/day for an adult human. However, various different dosages are described in scientific publications; see, for example, Ng et al. (1999) Food Chem. Toxicol. 37: 503-8; Ko et al. (1999) Arch. Phys. Med. Rehabil. 80: 964-7; Chen et al. (1999) Prostaglandins Other Lipid Mediat. 57: 99-111; and Thabrew et al. (1999) Ann. Clin. Biochem. 36: 216-20.

To determine the optimum concentration for any application, conventional techniques may be employed. Thus, for in vitro and ex vivo use, a variety of concentrations may be used and various assays employed to determine the degree of neuronal damage, such as, for example, measurements of cell death, infarct size, and cognitive dysfunction.

Formulations of the present invention adapted for oral administration may be presented as discrete units such as capsules, cachets or tablets each containing a predetermined amount of the active ingredients; as a powder or granules; as a solution or a suspension in an aqueous or non-aqueous liquid; or as an oil-in-water liquid emulsion or a water-in-oil liquid emulsion. The active ingredients may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethylcellulose), lubricant, inert diluent, preservative, disintegrant (e.g. sodium starch glycollate, cross-linked povidone, cross-linked sodium carboxymethylcellulose) surface-active or dispersing agent. Molded tablets may be made by moulding in a suitable machine a mixture of the powdered compound moistened with an inert liquid diluent. The tablets may optionally be coated or scored and may be formulated so as to provide controlled release of the active ingredients therein using, for example, hydroxypropylmethylcellulose in varying proportions to provide the desired release profile.

The subject compositions may be administered parenterally including intravascularly, arterially or venous, subcutaneously, intradermally, intraperitoneally, intraorganally, intramuscularly, or the like.

Formulations for parenteral administration include aqueous and non-aqueous isotonic sterile injection solutions which may contain buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

For topical administration, the subject compositions may be provided as a wide variety of product types including, but are not limited to, lotions, creams, gels, sticks, sprays, ointments and pastes. These product types may comprise several types of formulations including, but not limited to solutions, emulsions, gels, solids, and liposomes.

Compositions useful for topical administration of the compositions of the present invention formulated as solutions typically include a pharmaceutically-acceptable aqueous or organic solvent. The terms “pharmaceutically-acceptable organic solvent” refer to a solvent which is capable of having a gamma-, beta-, or delta-tocopherol composition and/or metabolite and/or derivative thereof, or mixtures thereof, dispersed or dissolved therein, and of possessing acceptable safety properties (e.g., irritation and sensitization characteristics). Examples of suitable organic solvents include: propylene glycol, polyethylene glycol (200-600), polypropylene glycol (425-2025), glycerol, 1,2,4-butanetriol, sorbitol esters, 1,2,6-hexanetriol, ethanol, isopropanol, butanetriol, sorbitol esters, 1,2,6-hexanetriol, ethanol, isopropanol, butanediol, and mixtures thereof.

If the topical compositions useful in the subject invention are formulated as an aerosol and applied to the skin as a spray-on, a propellant is added to a solution composition. Examples of propellants useful herein include, but are not limited to, the chlorinated, fluorinated an chloro-fluorinated lower molecular weight hydrocarbons.

Topical compositions useful in the subject invention may be formulated as a solution comprising an emollient. As used herein, “emollients” refer to materials used for the prevention or relief of dryness, as well as for the protection of the skin. A wide variety of suitable emollients are known and may be used herein.

Another type of product that may be formulated from a gamma-, beta-, delta; tocopherol enriched tocopherol composition and/or a gamma-, beta-, or delta-tocopherol metabolite enriched composition is a cream. Another type of product that may be formulated from a composition of the present invention is a lotion.

Yet another type of product that may be formulated from a composition of the present invention is an ointment. An ointment may comprise a simple base of animal or vegetable oils or semi-solid hydrocarbons (oleaginous). Ointments may also comprise absorption ointment bases which absorb water to form emulsions. Ointment carriers may also be water soluble.

Another type of formulation is an emulsion. Emulsifiers may be nonionic, anionic or cationic and examples of emulsifiers are described in, for example, U.S. Pat. Nos. 3,755,560, and 4,421,769.

Lotions and creams can be formulated as emulsions as well as solutions.

Single emulsions for topical preparations, such as lotions and creams, of the oil-in-water type and water-in-oil type are well-known in the art. Multiphase emulsion compositions, such as the water-in-oil-in-water type, are also known, as disclosed, for example, in U.S. Pat. No. 4,254,105. Triple emulsions are also useful for topical administration of the present invention and comprise an oil-in-water-in-silicone fluid emulsion as disclosed, for example in U.S. Pat. No. 4,960,764.

Another emulsion useful in the topical compositions is a micro-emulsion system. For example, such a system comprises from about 9% to about 15% squalane, from about 25% to about 40% silicone oil; from about 8% to about 20% of a fatty alcohol; from about 15% to about 30% of polyoxyethylene sorbitan mono-fatty acid (commercially available under the trade name TWEENS) or other nonionics; and from about 7% to about 20% water.

Liposomal formulations are also useful for the compositions of the present invention. Such compositions can be prepared by combining gamma-, beta, or delta-tocopherol, and/or metabolite thereof, and/or derivative thereof, and/or mixtures thereof, with a phospholipid, such as dipalmitoylphosphatidyl choline, cholesterol and water according to known methods, for example, as described in Mezei et al. (1982) J. Pharm. Pharmacol. 34:473-474, or a modification thereof. Epidermal lipids of suitable composition for forming liposomes may be substituted for the phospholipid. The liposome preparation is then incorporated into one of the above topical formulations (for example, a gel or an oil-in-water emulsion) in order to produce the liposomal formulation. Other compositions and pharmaceutical uses of topically applied liposomes are described for, example, in Mezei (1985) Topics in Pharmaceutical Sciences, Breimer et al. eds., Elsevier Science, New York, N.Y., pp. 345-358.

For rectal administration, the subject compositions may be provided as solutions for enemas, as suppositories with a suitable base comprising, for example, cocoa butter or a salicylate, or as other convenient applications.

Formulation for vaginal administration may be presented as pessaries, tampons, creams, gels, pastes, foams or spray formulations containing in addition to the active ingredient such carriers as are known in the art to be appropriate.

To determine the optimum concentration for any application, conventional techniques may be employed. Thus, for in vitro and ex vivo use, a variety of concentrations may be used and various assays employed to determine the degree of dysfunction of the cells when exposed to stress. Examples of such assays are described herein and have been described, for example, in U.S. Pat. No. 5,801,159.

The above-mentioned compositions and methods of administration are meant to describe but not limit the methods and compositions of the present invention. The methods of producing various compositions and devices are within the ability of one skilled in the art and are not described in detail here.

The gamma-, beta-, or delta-tocopherol enriched tocopherol compositions, and/or gamma-, beta-, or delta-tocopherol metabolite enriched compositions, and methods using the compositions are capable of reducing neuronal damage associated with cerebral ischemia. These conditions can be induced experimentally by chemical interference or by changing the environmental conditions in the laboratory (e.g., by inducing anoxia, hypothermia, hyperthermia, etc.).

Various assays, compositions and methods useful for identifying compositions and methods for reducing neuronal damage are provided in the Examples.

The following examples are provided to illustrate, but not limit, the invention.

### EXAMPLES

### Example 1

Example 1 describes cell-based assays for determining the ability of a tocopherol composition to counteract ischemic-induced neuronal cell injury and cell death.

Insults to the brain that disrupt its blood supply, as in ischemia, or its oxygen supply, as in hypoxia (low oxygen) or anoxia (zero oxygen), rapidly cause neuronal imbalance leading to cell death (Flynn et al. (1989) Ischemia and Hypoxia, pp. 783-810, In: Basic Neurochemistry, Siegel et al. (Eds.), Raven Press, N.Y.). Cerebral ischemic insults are modeled in animals by occluding vessels to, or within, the cranium (Molinari (1986) Experimental models of ischemic stroke, pp. 57-73, In: Stroke: Pathophysiology, Diagnosis and Management, Vol. 1, Barnett et al. (Eds.), Churchill Livingstone, N.Y.). In vitro models of ischemia use different means of oxygen and glucose deprivation. For example, by placing neuronal cultures into large anaerobic or hypoxic chambers and exchanging culture medium with oxygen-free and defined ionic composition media (Goldberg et al. (1990) Stroke 21:75-77). The toxic overstimulation of neuronal glutamate receptors, especially N-methyl-D-aspartate (NMDA) receptors, contribute to hypoxic-ischemic neuronal injury (Choi (1988) Neuron 1:623-634), ischemic induction of reactive oxygen species (ROS) (Watson et al. (1988) Ann. NY Acad. Sci. 59:269-281), excessive calcium influx (Grotta et al. (1988) Stroke 19:447-454), arachidonic acid increase (Siesjo (1981) J. Cereb. Blood Flow Metab. 1:155-185), and DNA damage (MacManus et al. (1993) Neurosci. Lett. 164:89-92) causing a cascade of neurodegeneration.

Among various types of primary neuronal cultures, primary embryonic hippocampal neuronal culture is widely used for several reasons. The hippocampus is a source of a relatively homogenous population of neurons with well-characterized properties typical of central nervous system (CNS) neurons in general. Pyramidal neurons, the principal cell type in the hippocampus, have been estimated to account for 85% to 90% of the total neuronal population (Banker et al. (1998) Culturing Nerve Cells, 2^{nd} edition, The MIT Press, Cambridge, Mass.). Also, the hippocampus exhibits a remarkable capacity for activity-dependent changes in synaptic function, such as long-term potentiation (Hawkins et al. (1993). Annu. Rev. Neurosci. 16:625-665).

Hippocampal cultures typically are prepared from 18- to 19-day fetal rats. At this age, the generation of pyramidal neurons, which begins in the rat at about E15, is generally complete. The tissue is easy to dissociate, the meninges are removed readily, and the number of glial cells still is relatively modest (Park et al. (2000) J. Neurochem 74:114-124).

1A. Primary Cell Culture

The following protocol describes the procedure used to isolate and culture primary hippocampal neuronal cells from embryonic rat brain for use in the cell-based assays described herein.

Prior to cell isolation, long tip Pasteur pipettes with an opening of 1 mm, 0.4-0.5 mm, and 0.25 mm were fire-polished, cleaned with 70% ethanol, siliconized (Sigmacote, Sigma Chemical Cat. No. SL-2) and autoclaved. All other instruments for dissection were soaked in 70% ethanol at least 2 hr before the dissection. Also prior to cell isolation, culture flasks (T75 cm²) and plates were coated with poly-D-lysine (Sigma Chemical, Cat. No. β-6407). For the coating, 50 μg/ml poly-D-lysine was added to the flask or plate (5 ml per T75 cm² flask and 50 μl/well in a 96 well plate) for one hour. The flask or plate was then washed twice with sterile, distilled water and allowed to air dry in a culture hood for one hour before use. HBSS (Ca-Mg free) was prepared as follows: 10.0 ml 10x HBSS (Hank's CMF—Gibco #310-4180), 3.3 ml 0.3 M HEPES, pH 7.3, 10 ml of 0.35% sodium bicarbonate, 1.0 ml Penicillin/Streptomycin (100X) and 1.0 ml 100 mM pyruvate were mixed with 74.7 ml H₂O to make 100 ml of solution.

A pregnant rat (El 8-El 9) was euthanized with CO₂, and the uterus was removed. The embryos were removed from the sac, decapitated and their brains were removed. The brains were immersed in cold (4° C.) BSS (Ca/Mg free) in a small petri dish. A dish (100-mm) was covered with paraffin to make a better surface for the dissection. The hippocampi were removed from the brains under a dissecting microscope and placed on the paraffin-covered dish. The meninges were stripped away and the dissected hippocampi were collected in a small petri dish in HBSS (Ca/Mg free).

The hippocampi from one litter were placed in a 15-ml centrifuge tube (generally 10-12 brains/litter), and the tube was filled with HBSS (Ca/Mg free). After centrifugation at 1000 rpm for 2 min using the desktop centrifuge, the supernatant was removed. 2 ml of HBSS (Ca/Mg free) was added to each tube and the tissue was triturated 2 times with a long tipped siliconized pipette with the three different opening sizes (total of 6-7 times). The trituration started with a pipette with a normal opening size, and then smaller (half of size), then one with the smallest hole. After centrifugation at 1000 rpm for 2 minutes, the supernatant was discarded and 2 ml of Neurobasal/B27i (with antibiotics) was added to each tube. Neurobasal/B27i media contains Neurobasal medium (Life Technologies Cat No. 21103-049) with 1×B27 supplement (Life Technologies Cat No. 17504-044), 0.5 μM L-glutamine, 25 μM L-glutamic acid, and 1×Penicillin/Streptomycin. The cells were triturated 1 time with a long tip siliconized pipette with three different opening sizes. The trituration started with a pipette with a normal opening size, then the smaller one (half of size) and finally the one with the smallest hole. Cell density was determined in a hemocytometer using the trypan blue exclusion method. A stock solution of 0.4% trypan blue in 0.9% NaCl was mixed one to one with a few drops of the cell suspension, and allowed to stand 4 minutes before counting the fraction of dye-excluding cells. A typical yield is 3×10⁵-6×10⁵ cells/brain.

The desired number of viable cells were added to poly-D-lysine-coated 12-well plates, flasks or MetTek dishes in Neurobasal/B27i, and incubated in air atmosphere with 5% CO₂ at 37° C. The cells were generally seeded at a density of 1.5×10⁶ cells per T75 cm² flask and at a density of˜100,000 cells per well of a 12-well plate. Each T75 cm² flask received 15 ml of medium and each well of a 12-well plate received 1 ml of medium.

After three to four days in culture, half the media was removed from each well or flask, and an equal amount of fresh Neurobasal/B27m medium (Neurobasal medium with 1×B27 supplement, 0.5 μM L-glutamine), which contains 5 μM cytosine arabinoside (AraC), was added. Seven to eight days from the initial culture, half the media was removed from each well or flask, and an equal amount of fresh Neurobasal/B27m medium (no Ara-C) was added.

1B. Cell Injury Assay

In the following assay, ischemia is induced by anoxia-reoxygenation in cultured hippocampal neuronal cells and test agents are accessed for their potency and/or efficacy against ischemic-induced neuronal cell injury and cell death. The assay protocol is diagramed as follows:

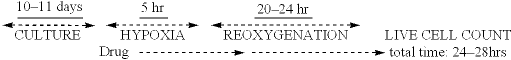

Primary hippocampal neuronal cells were prepared and plated on poly-D-lysine coated 12-well plates as described in Example 1A above. The cells were cultured for 10-11 days as described in Example 1A.

100 ml of LoG-Neurobasal medium in a T150 cm² flask was pre-equilibrated in the hypoxic chamber overnight and 20 ml of LoG-Neurobasal medium in a T75 cm² flask was pre-equilibrated in a standard incubator (5% CO₂) overnight. LoG-Neurobasal medium contains NoG-Neurobasal medium (no glucose) (Life Technologies, custom order) plus 0.5 mM glucose, 0.5 mM L-glutamine and 0.25×Penicillin/Streptomycin. 100 ml of Neurobasal/B27AO medium in a T150 cm² was pre-equilibrated in a standard incubator (5% CO₂) overnight. Neurobasal/B27AO medium contains Neurobasal medium (Life Technologies, Cat. No. 21103-049) with 2×B27 minus AO supplement (Life Technologies, Cat. No. 10889-038), 0.5 mM L-glutamine, and 0.25×Penicillin/Streptomycin.

The equilibrated 100 ml of LoG-Neurobasal in T150 cm² flask was removed from the hypoxic chamber, and the medium was lightly bubbled with 100% N₂ for 30 min. to deoxygenate completely. Existing culture medium (Neurobasal/B27m) was aspirated from the cultured cells in each 12-well plate using the vacuum pump with an attached sterile glass pastuer pipette. The cells were washed once with 2 ml of glucose free-BSS₀ (pH 7.4). Glucose free-BSS₀ (pH 7.4) contains 143.6 mM NaCl, 5.4 mM KCl, 1.8 mM CaCl₂, 0.8 mM MgSO₄, 1 mM NaH₂PO₄, 26.2 mM NaHCO₃, 10 mg/l phenol red and 0.25×Penicillin/Streptomycin.

The cultured neurons (10-11 days from initial culture) were replenished with deoxygenated LoG-Neurobasal (1 ml per well for each well of a 12-well plate). The test agents were added directly to each well (usually 3 concentrations of the compound plus positive control, each in triplicate). Generally, the test agents were dissolved in 100% DMSO. To reduce the DMSO effect on the cells, highly concentrated compounds were added in a small quantity (typically 200×concentration). The concentration of DMSO in the culture did not exceed 0.5%.

The plates were placed, with their lids left ajar, in the anaerobic chamber for 5 hours. For normoxia controls, pre-equilibrated normoxic LoG-Neurobasal medium was added to each well and the plate replaced in the standard incubator (5% CO₂) for 5 hours. After 5 hours of hypoxia, the culture media was carefully aspirated and 2 mL of new oxygenated (pre-equilibrated) Neurobasal/B27AO was added to each well. Reoxygenated medium was achieved by placing medium overnight in the culture incubator. The same test agents with same the concentrations were added back into the corresponding wells. The plates were placed in the cell culture incubator and reoxygenated for 20-24 hours. After reoxygenation for 20-24 hours, the number of live neurons were counted using the cell tracker green fluorescence method as follows. The culture medium was aspirated from each well of the 12-well plates and the neurons were washed once with 2 ml of HBSS (prewarmed to 30-37° C.; 25 mM HEPES, 100 mM NaCl, 5 mM KCl, 1.2 mM MgCl₂, 1.3 mM CaCl₂, 1.0 mM KH₂PO₄, pH 7.4, filter sterilized). 1 ml of 5 μM Cell Tracker Green fluorescent dye (Molecular Probes, Cat. No. 2925) dissolved in HBSS was added to the cells and the plates were placed in the dark at room temperature for 15 minutes. After washing the neurons once with 2 ml of HBSS, 1 ml of HBSS was added to each well, and fluorescent cells were counted using the fluorescent microscope.

In experiments carried out in support of the invention, gamma-tocopherol enriched tocopherol compositions and gamma-CEHC enriched compositions each provided at least 40% protection against hippocampal cell injury in the primary hippocampal cell model.

### Example 2

### Animal Cerebral Infarct Assay

This assay is used to assess the efficacy of the test agents in protecting the brain against necrosis following cerebral ischemia induced in rats. Middle Cerebral Artery Occlusion (MCAO) is a widely used technique to induce transient focal cerebral ischemia in animal models. It has been demonstrated that the rat model of MCAO is an appropriate approximation of ischemic damage in humans. Furthermore, this model accurately represents the involvement of middle cerebral artery (MCA), the most affected vessel in human stroke, and also allows reperfusion as it happens in humans. MCAO by a two-hour occlusion is used to produce the maximum size of cortical infarction without increased mortality at twenty-four hours.

2A. Middle Cerebral Artery Occlusion (MCAO)

Male Wistar rats (Hadan, Ind.) weighing 300-350 g were allowed free access to water and commercial rodent diet under standard laboratory conditions. The room temperature was maintained at 20-23° C. and room illumination was on a 12/12-hour light/dark cycle. The rats were acclimatized to the laboratory environment 5 to 7 days prior to the study.

Before surgery, the animals were fasted overnight, but had free access to water. The rats were anesthetized with 3.0% isoflurane (Aerrane, Front Dodge, Iowa) in 0.8% oxygen and the animal's neck was shaved and sterilized before the operation. The animals were subjected to two hours MCAO using a modified intraluminal filament technique. A midline incision on the ventral part of the neck was made to expose external and internal carotid arteries. The right external and common carotid arteries were ligated by a suture (silk 5/0, Carlisle Laboratories, Farmers Branch, Tex.) and the right internal artery was temporarily ligated by a microvascular clip (Fine Science Tool Inc., Foster City, Calif.). A small incision was made in the common carotid artery and a nylon filament, its tip rounded by heating, prepared from a fishing line (Stren Fishing Lines, Wilmington, Del.) was inserted from the right common carotid artery. The filament was advanced into the internal carotid artery 18-20 mm from the point of bifurcation of internal and external arteries and a suture was tightly ligated around the filament. Two hours post occlusion, the animals were re-anesthetized to allow reperfusion for the remaining of the experiment by removal of the filament.

2B. Drug Administration

The following is a description of various ways by which gamma-tocopherol enriched tocopherol compositions and gamma-tocopherol metabolite enriched compositions are administered in this assay.

2B1. I.C.V. Infusion

The anesthetized animal were placed on a stereotaxic apparatus (Harvard Apparatus, S. Natick, Mass.). Anesthesia was maintained by inhalation of 3.0% isoflurane in 0.8% oxygen throughout the entire procedure. The scalp was shaved and sterilized prior to surgery. A midline sagittal incision about 3 cm long was made slightly behind the eyes to expose the skull. The skull was scraped with a rounded end spatula to remove periosteal connective tissue. A bur hole was placed 1.5 mm lateral, 1 mm posterior to the left of the bregma to mark the left lateral ventricle. A brain infusion cannula (ALZET CO. Palo Alto, Calif.) was inserted 4 mm deep into the hole. The desired depth was adjusted by attaching spacers to the cannula. The cannula was attached to a 4-cm silastic catheter (Helix Medical Inc.) fixed in place with dental cement (Ketac-cement, Norristown, Pa.). The catheter was either attached to a primed osmotic pump placed subcutaneously between the shoulder blades for permanent infusion or to a syringe for a short infusion.

2B2. I.V. Osmotic Pump Implantation into the Jugular Vein

Anesthesia is maintained by inhalation of 3.0% isoflurane in 0.8% oxygen throughout the entire procedure. The animal's neck is shaved and sterilized before operation. A midline incision is made on the ventral part of the neck to exposes the jugular vein. The vein is isolated and ligated with a suture (silk 5/0, Carlisle Laboratories, Farmers Branch, Tex.) rostral to the point of the incision and a microvascular clip (Fine Science Tool Inc., Foster City, Calif.) close to the heart. A small incision is made between two ligations. A 2 cm silastic catheter (Helix Medical Inc) attached to a PE-60 tube (Becton. Dickinson and Co., Sparks, Md.) connected to an ALZET (ALZET CO. Palo Alto, Calif.) pump is introduced and advanced 2 mm into the jugular vein toward the heart, the microvascular clip is removed and the catheter is secured in place with a suture (silk 5/0, Carlisle Laboratories, Farmers Branch, Tex.). The pump is placed into a pocket made subcutaneously between the shoulder blades, allowing the catheter to reach over neck to the jugular vein with sufficient slack to permit free movement of neck and head.

2B3. I.V. Infusion via Femoral Vein

Anesthesia was maintained by inhalation of 3.0% isoflurane in 0.8% oxygen throughout the entire procedure. The exterior site of the right femoral vein was shaved and sterilized prior to surgery. A 3 cm incision was made in the right groin region and the femoral vein was isolated. A small incision was made on the femoral vein temporarily ligated with a microvascular clip to introduce and advance a polyethylene (PE-50) catheter (Becton. Dickinson and Co., Sparks, Md.). The catheter was secured in place with suture (silk 5/0, Carlisle Laboratories, Farmers Branch, Tex.). The other end of the catheter was attached to a syringe filled with the heparinized saline for the bolus injection. Using a hemostat a pocket was made subcutaneously on the back of the animal so the PE catheter can be brought up to the exteriorization point at the nape of the neck for either a bolus injection or a continuous injection by an osmotic pump.

2B4. I.P. Injection

An awake rat is held in a standard hand hold position, a 23 ¾ G needle is injected into the lower right quarter of the abdomen pass the peritoneum, slightly off the midline. To avoid organ injection, the plunger of the syringe is slightly pulled back. If no fluid is withdrawn, the content of the syringe is delivered into the abdominal cavity.

2B5. Gavage Feeding

A standard rat gavage tube (Popper & Sons Inc, New York) was attached to a 3-cc hypodermic syringe. The animal was held by the shoulder in a vertical position. The feeding tube was placed into the mouth then advanced until it reached the stomach (the approximate insertion length of the tube was measured prior to the feeding). The content of the syringe was slowly delivered, and then the tube is withdrawn.

2C. Behavioral Assessment

One hour after MCAO, the animal is gently held by its tail and observed for forelimb flexion. Then the animal is put on the floor and observed for walking pattern. Only an animal(s) scoring 3 on Bederson grading system (Table 1) is used for this study.

TABLE 1
Bederson grading system for neurological evaluation

| Neurological deficit | Grading | Behavioral observation |
|---|---|---|
| Normal | grade 0 | No observable deficit |
| Moderate | grade 1 | forelimb flexion |
| Severe | grade 2 | forelimb flexion, decreased resistance to |
| | | lateral push |
| | grade 3 | forelimb flexion, decreased resistance to |
| | | lateral push, circle to paretic side |

2D. Temperature Control

Body temperatures are controlled and maintained at 37.5° C.+/−1 degree via external heating and cooling devices. To lower the body temperature, animals are located in a cooling chamber which uses ice to cool circulating air. Throughout the study, the body temperature is recorded using a temperature transponder (BMDS Inc., Seaford, DL) implanted subcutaneously at the time of MCAO between the rat shoulder blades and the body temperature is read via a pocket scanner (BMDS Inc., Seaford, DL). Alternatively, the body temperature is taken by inserting the temperature probe into the animal's rectum. The body temperature is recorded every hour for 6 hours post occlusion; however, body temperatures are taken more frequently so that the animal is maintained at the normothermic temperature.

2E. Evaluation of Ischemic Damage

The animals are sacrificed by CO₂ asphyxiation (dry ice) 24 hours post-MCAO. The skull is removed to expose the brain using a small bone cutter and starting from the base of the skull. The brain is removed quickly, rinsed in a chilled saline and placed on a rat brain tissue slicer (ASI instrument, Michigan). Seven 2-mm thick coronal slices are cut from each brain using razor blades. The slices are immersed in 0.9% saline containing 1.0% 2,3,5-triphenyltetrazolume chloride (TTC) (Sigma Chemical Co., St. Louis, Mo.) and incubated in a 37° C. water bath for 30 minutes.

After staining, each 2-mm slice is photographed with a TMC-7 camera (JH Technologies, Calif.) and the image is captured and stored on a computer. This image is used for the measurements of the regions of interest using a computer-based image processing system (Metamorph).

To measure each area, the region of interest is selected using a freehand selection tool and the area is computed by selecting the measure command. The measurements for primary regions of interest are right hemisphere, left hemisphere, total infarct, subcortical infarct, total penumbra and subcortical penumbra. After all regions of interest are measured for all seven slices of the brain, they are sorted by slice number and the corresponding regions of interest using an Excell macro, Statistic Final. This macro also calculaes the cortical penumbra, cortical infarct and total ischemic damage for each slice then the corresponding areas of each rat brain are added together to produce a single measurement for each area. Since the ipsilateral hemisphere is swollen following MCAO, edema volume is calculated and reported as the volumetric differences between the right and left hemispheres of each brain slice. Using the percentage of hemispheric swelling, all the volumes are corrected for the edema.

The volume of the damage is determined using the calculations for each rat's brain as follows.

Cortical Penumbra: Total Penumbra−Subcortical Penumbra

Cortical Infarct: Total Infarct−Subcortical Infarct

Total Ischemic Damage: Total Penumbra+Total Infarct

Summary (mm²): measurements of each column are added together.

Total Volume (mm³): The sum of each column is multiplied by 2 (the thickness of the tissue).

Edema Volume: The volumetric differences between the sum of right and left hemispheres determine the edema volume.

% Hemispheric swelling (H.S.): Edema×100/left hemisphere

Correction for edema for each measurement:

T.P. (Total Penumbra) corrected=T.P.−(T.P.×% H.S./100)

S.P. (Total Penumbra) corrected=S.P.−(S.P.×% H.S./100)

C.P. (Total Penumbra) corrected=C.P.−(C.P.×% H.S./100)

T.I. (Total infarct) corrected−T.I.=(T.I.×% H.S./100)

S.I. (Total infarct) corrected=S.I.=(S.I.×% H.S./100)

C.I. (Total infarct) corrected=CL=(C.I.×% H.S./100)

T.I.D. (Total Ischemic Damage) corrected=T.I.D.−(T.I.D.×% H.S./100)

The sample size is chosen to achieve a 90% probability of significant results. The measurements, which represent the same region of interest in seven slices of each rat's brain are added together to yield a single measurement for total infarct, subcortical infarct, cortical infarct, total penumbra, subcortical penumbra, cortical penumbra, total ischemic damage and edema in each animal. Group data are presented as means +/−SEM and p<0.05 are considered significant. A comparison of each region of interest between groups is carried out by unpaired student t test (between two groups) or one way ANOVA followed by post hoc Bonferroni's multiple comparisons or by the nonparametric Dunnett's test (between control and the drug treated groups).

2F. Results

When gamma-tocopherol enriched tocopherol composition (greater than 90% gamma-tocopherol) was administered I.V. at the time of MCAO, total infarct volume, total ischemic damage and edema were significantly reduced relative to that of control animals. Administration of the gamma-tocopherol metabolite, gamma-CEHC, I.V. at the time of MCAO also resulted in significantly reduced total infarct volume and total ischemic damage relative to that of controls. Administration of gamma-CEHC at the time of MCAO also resulted in reduced tissue edema relative to that of control animals. Thus, administration of a gamma-tocopherol enriched composition or a gamma-tocopherol metabolite enriched composition provided protection to the brain against damage and effects associated with cerebral ischemia.

Administration of a gamma-tocopherol enriched composition or a gamma-tocopherol metabolite enriched composition at the time of reperfusion resulted in reduction of total infarct volume, total ischemic damage and tissue edema relative to that of controls.

The protective effects of alpha-tocopherol and delta-tocopherol were also assessed in the MCAO assay. At the same concentration, gamma-tocopherol was more effective in the reduction of total infarct volume and in the reduction of tissue edema than alpha- or delta-tocopherol. Delta-tocopherol was more effective in the reduction of total infarct volume than alpha-tocopherol.

### Example 3

### Animal Assay for Behavioral Recovery after Cerebral Ischemia

This assay is used to assess the efficacy of the test agents in behavioral recovery after cerebral ischemia induced in rats. Clinical behavior evaluation includes neurological examination, sensomotor activity and learning and memory behavior testing.

Three groups of animals, sham treated, MCAO animals treated with test agents and MCAO animals treated with control vehicle, are used. The time points of testing is dependent on the individual test.

Male Wistar rats are treated and MCAO is performed as described in Example 2A. Polyethylene catheters are inserted into a jaguar vein or femoral vein for blood sampling and drug administration.

After the animals regain consciousness, they are placed in the cooling system or refrigerator for cooling down the body temperature. The temperature in the cooling system or refrigerator is kept between 6 to 14 C°. The animal body temperature is maintained at 37.5±1 C° during the operation and 24 hours post-ischemia reperfusion. Animal body temperature is recorded before the operation, during the operation and at 15, 30, 60, 90, 120, 180, 240 minutes and 24 hours post-operation.

The neurological status of each rat after MCAO is evaluated within 1, 4, 6, and 24 hour after induction of ischemia. Rats that extended both forelimbs toward the floor when held by the tail and that had no other neurological deficit are assigned a grade of zero. Rats with consistent forelimb flexion and which bent to the contralateral side when held by the tail, but no other abnormalities are graded 1. Rats that have consistently reduced resistance to lateral push and gait toward the paretic side are graded 2. Rats that circle toward the paretic side consistently within 1 hour after MCAO are graded 3. Animals which do not get grades of 2 or 3 are excluded from the study.

The test agents and controls are given by i.p. or i.v. bolus and/or continuous infusion as described in Example 2.

3A. Sensorimotor Behavior

Fore and Hindlimb Grip Strength Test in Rats

The animals (sham treated, MCAO animals treated with test agents and MCAO animals treated with control vehicle) are moved into the testing room 30 minutes before testing. A Computerized Grip Strength Meter for Rats (Dual stand Model, Columbus Instruments, USA) is used to measure grip strength. Prior to testing, each gauge is calibrated with a set of known weights and the apparatus adjusted for the size of animal (about ½ inch clearance on both side of the animal). The forelimb measurements are done with the meter in the tension peak mode to freeze the reading as the subject is pulled away from the grip bar. The hindlimb measurements are done with the meter in the compression peak mode to freeze the reading as the subject's hindlimbs are pulled over the bar toward the meter. The animal is hand-held by the investigator as it is pulled past the grip bars. The animal is held around the midsection, with the fingers and thumb curling under the body and the index finger on top, over the back and shoulders, with the tip of the finger by the animal's neck. Alternatively, the animals are grasped by the scruff of the neck with one hand and at the base of the tail with the other. Both methods of holding the animals leave the fore and hind limbs free to grasp the grip bars as the animal is moved past them. The animals are handled consistently. Typically, three successive readings are taken for each animal with an inter-trial interval long enough to record the data and zero both meters for the next trial.

Beginning on post-operative day 2, animals are given a test that assesses sensorimotor integration and testing is continued twice weekly until day 16 after MCAO operation. The studies are carried out in blinded-randomized fashion.

Treadmill Test in Rats

The animals (sham treated, MCAO animals treated with test agents and MCAO animals treated with control vehicle) are moved into the testing room 30 minutes before testing. A Rota-Rod Treadmill for Rats (7750 Accelerating Model from UGO BASILE, COMERIO-ITALY) is used to test ability to perform on a treadmill. Each rat has 2-3 training runs of 1-2 minutes at intervals of 2-3 hours before testing. The cylinder is set in motion before placing the rats in position. The rotor is started at a constant selected speed and the rats are placed, one by one, in their sections, at the same time, the trip counter is reset to zero. The rats are placed on the treadmill in a manner that facilitates walking while minimizes struggling. Data acquisition from the treadmill occurs through the use of connections to BASILINK Cat. 2000 or to MINILINK Cat. 2500. The BASILINK is a modular self-sufficient Data Acquisition System that enables the gathering of a list in appropriate files and printing of experimental data generated by one or more instruments. The MINILINK is a compact, non-printing version of the Basilink, suitable for the processing of data generated by a limited number of instruments via a computer.

Beginning on post-operative day 2, animals are given this test and testing is continued twice weekly until day 16 after MCAO operation. The studies are carried out in blinded-randomized fashion.

3B. Evaluation of Ischemic Damage

Examination for brain tissue damage (infarct size) is performed as described in Example 2E.

### Example 4

### Myocyte Viability Assay

4A. Isolation and Culture of Primary Neonate Myocytes

Materials

10×Heart Dissection Solution (HDS) contains the following components (g/l) in tissue grade water: NaCl, 68; HEPES, 47.6; NaH₂PO₄, 2; Glucose, 10; KCl, 4; MgSO₄, 1, pH adjusted to 7.4. Prior to filter sterilization of diluted (1XHDS) solution, 10 mg phenol red is added to each 500 milliliters of medium.

Transferrin and Bovine Insulin are obtained from Life Technologies, and resuspended at a concentration of 4 mg/ml in tissue culture grade water.

DMEM-F12-DMEM/F12, powder, 1:1 containing glutamine and pyridoxine hydrochloride is purchased from Life Technologies. To one liter equivalent of the powder is added 2.43 g of sodium bicarbonate and 10 ml of 100×Penicillin/Streptomycin in 950 ml of tissue culture grade water with stirring. The pH is adjusted to 7.2 with 1M HCl and volume is adjusted to 1 liter. The solution is filter sterilized then 2.5 ml of 4mg/ml Transferrin, 250 μl 4 mg/ml Insulin and 30.7 mg of bromodeoxyuridine are added.

DMEM-F12 is also prepared 4% FBS for pre-coating the tissue culture plates and initial suspension of the cardiomyocyte pellet.

Collagenase solution-49 mg of collagenase is resuspended in 120 ml 1×HDS.

Preparation of Primary Neonatal Myocyte Cultures

Tissue culture ware is pre-coated with DMEM-F12-4% FBS by incubating 50 μl per well of a 96-well plate and 0.25 ml per 12-well plate at 37° C.

Two-day old rat pups are removed from their mothers and placed in a sterile container. Pups are dipped quickly into 70% alcohol, then decapitated and the body is placed in an empty sterile tissue culture dish. An incision is made starting at the neck and progressing towards the belly, cutting through the sternum. The heart is removed and placed in a tissue culture dishes containing 1×HDS. The atria are trimmed, and the remaining ventricles are placed into a separate tissue culture dish containing 1×HDS, where they are sectioned into 3-4 pieces each. Ventricles are then transferred to a sterile 250 ml glass flask and the 1×HDS is removed. Twenty milliliters of pre-warmed collagenase solution are added to the ventricles, followed by incubation at 37° C. with shaking. After 30 minutes, the collagenase solution is removed and replaced with 20 ml fresh pre-warmed collagenase. Incubation is continued for an additional 30 minutes. At the end of the incubation, any tissue chunks are allowed to settle prior to removing the collagenase (containing the isolated cardiomyocytes) from the disrupted tissue pieces. The isolated myocytes are added to a 50 ml Falcon tube containing 2 ml Fetal Bovine Serum (FBS). The remaining tissue pieces are subjected to a second digestion by adding 20 ml fresh pre-warmed collagenase and incubating as above for 30 minutes. This second digest is then centrifuged at 1000 rpm for 10 minutes (tabletop centrifuge). The resulting supernatant is discarded, and the cell pellet is suspended with 4 ml FBS. The resulting cell suspension is placed in the incubator at 37° C. This step is repeated several additional times to harvest additional material.

Percoll gradients are prepared by adding 2.5 ml of 10×HDS to 22.5 ml of Percoll (Life Technologies) with mixing (Percoll Stock). Top Gradient solution (11 ml Percoll Stock and 14 ml 1×HDS) and Bottom Gradient solution (13 ml Percoll Stock and 7 ml 1×HDS) are prepared. Four milliliters of the Top Gradient solution are transferred into 6×15 ml sterile Falcon tubes. Three milliliters of the Bottom Gradient solution are placed in each tube by inserting a serological pipette to the bottom of the tube and slowly adding the liquid.

All the digests (5) are pooled in one 50 ml Falcon tube and centrifuged on a tabletop centrifuge at 1000 rpm for 10 minutes. The supernatant is discarded, and the cell pellet is resuspended in 12 ml of 1×HDS. Two milliliters of the cell suspension is added to the top of each gradient. The gradient tubes are then centrifuged at 3000 rpm for 30 minutes without braking in a Beckman Allegra 6 centrifuge (GH 3.8A rotor). Following centrifugation, the cells segregate into two sharp bands at the two interfaces. The lower band of the two bands is enriched for cardiomyocytes; there is also a cardiomyocyte pellet at the bottom of the tube. The upper band is enriched for fibroblasts and other non-cardiomyocytes. The upper portion of the gradient is aspirated down to just above the cardiomyocyte layer. The cardiomyocyte layer is then carefully removed along with the pellet, and the two fractions are pooled in a sterile 50 ml Falcon tube, along with corresponding fractions from additional gradient tube; then 1×HDS is added to a total volume of about 50 ml. The tube is centrifuged at 1000 rpm for 7 minutes. The supernatant is discarded and resuspended in 25 ml 1×HDS. A further 25 ml of 1×HDS is added and the centrifugation step is repeated. The cell pellet is resuspended carefully but thoroughly in 40-50 of DMEM/F12-4% FBS.

A small aliquot of the cell suspension is counted in a hemocytometer. The DMEM/F12-FBS coating medium is aspirated from the tissue culture dishes. The cardiomyocytes are added to the dishes at a plating density of 7.5×10⁴/well per 96-well in 200 μL and 1.5×10⁵/well per 12-well in 3 ml. The cultures are incubated at 37° C. with 5% CO₂ overnight. The original medium is removed, and add fresh DMEM/F12-5% FBS is added to each culture, prior to incubation at 37° C. with 5% CO₂ for a further 48 hours, before use.

Physiological ischemia is simulated by placing the cardiomyocytes in an anaerobic chamber (0% O₂, 85% N₂, 5% CO₂ & 10% H₂) in DMEM containing 1 mM glucose. Positive control cells are treated with DMEM-F12 containing 25 mM Glucose, which protects against the anoxia.

The test compounds are made up in DMEM-1 mM glucose in 96 deep-well mother plates and appropriately diluted for use in the assay. The media is removed from the cells and replaced with 200 μl of either DMEM-F12 or 1 mM DMEM with or without test compounds. The plates are then placed inside the 37° C. incubator in the anaerobic chamber and incubated for 16 hours. The plates are then removed and reoxygenated by the addition of DMEM-F12. The DMEM with or without test compounds is carefully removed from the cells and replaced with pre-warmed DMEM-F12 containing 5% FBS. Since the anoxic treatment may damage and/or kill the cells, causing them to dislodge from the bottom of the wells gentle aspiration of media is required at this step. The cells are then placed in a normal incubator at 37° C. and incubated for two hours to allow the cells to reoxygenate before assessing cell viability using, for example, the SYTOX Measurement assay or functional activity, using, for example the Contractility assay, both of which are described below.

4B. SYTOX Measurement

SYTOX Green nucleic acid stain is a high-affinity nucleic acid stain that easily penetrates cells with a compromised plasma membrane but will not cross the membrane of live cells. The nucleic acids of dead cells fluoresce bright green at 524 nm when excited with the 488 nm spectral source or with any other 450-500 nm source. Hence during a time course with cells incubated with SYTOX, the fluorescent emission at 524 nm is proportional to cell death. In the cell viability assay chemical ischemia is created in cells that are incubated with the SYTOX dye. The compound antimycin is used to simulate ischemia at the same time as stimulating the cells with forskolin. Antimycin inhibits electron transport from FADH₂ between complexes II and III and hence lowers cellular ATP synthesis. The assay is performed on cells grown in 96-well format allowing high throughput screening of compounds.

Materials: (i) SYTOX Green dye, supplied as a 5 mM solution in DMSO (Molecular Probes #S-7020) is stored at −20° C. until use. ii.) Antimycin-A: (Sigma #A-8674) dissolved 5.28 g in 10 ml of DMSO to make a 1 mM stock solution. Aliquot at 100 μl. This 10 mM stock is then diluted to 1 mM with DMSO and aliquoted at 25 μL. Both the 10 mM and the 1 mM are stored at −20° C. until use. (iii) Forskolin (Sigma #F-6886), dissolve 50 mg aliquot in 1.62 ml of DMSO to make a 100 mM stock solution. Aliquot at 100 μl and store at −20° C. until use. iv.) HBSS-SYTOX-FORSKOLIN-ANTIMYCIN (H-S-F-A) solution: H-S-F-A solution is made up fresh prior to each assay in sterile 175 ml or 225 ml Falcon bottles. The working concentrations for the assay are 3μM SYTOX, 50 μM Forskolin and 30 μM Antimycin.

In each experiment, a 1:500 dilution with HBSS from the 500 μM stock solution is made to generate a 1 μM final working solution of SYTOX Green. The final concentration of DMSO is 0.2%.

Cells are grown in 96-well black plates (with or without clear bottoms) at a density of 1000 cells per well for 24 hours. The necessary number of 96-well plates containing the specific cell type are obtained and placed in the laminar flow safety cabinet. A sterile microtiter basin is filled with the appropriate volume of pre-warmed 1×HBSS. Using aseptic technique and an 8-channel aspirator, the media is carefully removed from the cells and replaced with 200 μl of 1×HBSS. This is done as quickly as possible to prevent the cells drying out. The plates are then placed in the humidified 37° C. incubators of the Biomek 2000 Side Loader. Four plates are washed at a time so as to minimize the time that the cells are sitting in 1×HBSS prior to addition of the H-S-A-compound test solution.

Compound(s) are mixed with the 1 μM SYTOX working solution and added to the washed cell culture wells. Fluorescence is measured at designated time points (488 nm excitation and 530 nm emission). All of the sample readings subtract the background readings, which is cell-free wells only containing HBSS buffer with SYTOX green dye. The data is compared to non-treated cells (control) and is expressed as mean±S.D. from 4 culture wells. SYTOX Green fluorescence units obtained from each group are corrected for basal fluorescence (cell-free wells with only HBSS and fluorescence reagent). Data are represented as mean±SD. A paired t-test is performed comparing treated groups (Toxin+Agent) with non-treated groups (Toxin only) after subtracting the control groups (HBSS only) for each data set. Comparisons among groups are statistically evaluated by two-tailed t-test (Graphpad Prism Software, version 2.0). Differences are considered significant at P<0.05.

4C. Contractility Assay

Materials

Complete DMEM-F12: DMEM/F12, powder, 1:1 containing glutamine and pyridoxine hydrochloride is purchased from Life Technologies (Invitrogen Life Technologies, Carlsbad, Calif.). Powder sufficient to prepare one liter of buffer and 2.43 g of sodium bicarbonate is mixed into 950 ml of tissue culture grade water. The pH is adjusted to 7.2 with 1M HCl and the remaining water is added to make 1 liter. Following filter sterilization, 10 ml of 100×Penicillin/Streptomycin, 2.5 ml of 4 mg/ml Transferrin, 250 μl 4 mg/ml Insulin and 30.7 mg of bromodeoxyuridine are added, and the mixture is incubated at 37° C. prior to use.

1 mM glucose in DMEM is made from DMEM without L-glutamine, without glucose, without sodium pyruvate, purchased from Life Technologies.

20 μM Fluo-4: Cell permanent AM ester of Fluo-4 is obtained from Molecular Probes (Eugene, Oreg.) as a dry powder to be stored at −20° C. This fluorescent dye is light sensitive and should be made up fresh at 1 mM in DMSO prior to use to prevent light degradation.

10 mM CaCl₂ solution is made fresh each day in 1×HBSS and incubated at 37° C. prior to use.

Neonatal cardiomyocytes are isolated as described above. The cardiomyocytes are plated in 96-well format (black clear-bottomed plates) at a density of 7.5×10⁴ per well and grown for 2 days in the presence of 5% FBS prior to use in the assay.

Physiological ischemia is simulated by placing the cardiomyocytes in an anaerobic chamber (0% O₂, 85% N₂, 5% CO₂ & 10% H₂) in DMEM containing 1 mM glucose. Positive control cells are treated with DMEM-F12 containing 25 mM Glucose, which protects against the anoxia.

The test compounds are made up in DMEM-1 mM glucose in 96 deep-well mother plates and appropriately diluted for use in the assay. The media is removed from the cells and replaced with 200 μl of either DMEM-F12 or 1 mM DMEM with or without test compounds. The plates are then placed inside the 37° C. incubator in the anaerobic chamber and incubated for 16 hours. The plates are then removed and reoxygenated by the addition of DMEM-F 12. The DMEM with or without test compounds is carefully removed from the cells and replaced with pre-warmed DMEM-F12 containing 5% FBS. Since the anoxic treatment may damage and/or kill the cells, causing them to dislodge from the bottom of the wells gentle aspiration of media is required at this step. The cells are then placed in a normal incubator at 37° C. and incubated for two hours to allow the cells to reoxygenate.

A working solution of 20 μM Fluo-4 is added to pre-warmed 1×HBSS. The cells are loaded with Fluo-4 by first removing media from the cells and replacing with 100 μl of 20 μM Fluo-4. Unloaded control cells are treated in parallel with 1×HBSS alone. All cells are then incubated at 37° C. for 30 minutes. Before fluorescence measurements are made, the cells are washed in indicator-free medium (HBSS) to remove any dye that is non-specifically associated with the cell surface. Cells are then incubated for an additional 20 minutes at room temperature. Basal Fluo-4 fluorescence is measured using the 485 nm excitation and 538 mn emission filter pair on a microplate flourometer (Fluorskan™, Thermo Labsystems Oy, Helsinki, Finland). Each well is read for 160 ms to obtain a baseline reading, then stimulated to contract by addition of 10 nM CaCl₂. Following incubation at 37° C. for 30 minutes, a stimulated fluorescence reading is taken after 90 minutes.

### Example 5

### Model of Myocardial Infarction: Left Coronary Ligation (Rat)

Male Sprague-Dawley weighing 250-320 g are allowed free access to water and commercial rodent diet under standard laboratory conditions. Room temperature is maintained at 20-23° C. and room illumination is on a 12/12-hour light/dark cycle. Animals are acclimatized to the laboratory environment 5 to 7 days prior to the study and are fasted overnight prior to surgery.

Surgical Procedure for Acute Studies

Rats are anaesthetized with Urethane (1.2-1.5 gm/kg). Core body temperature is maintained at 37° C. by using a heating blanket. The surgical area is shaved, and a ventral midline incision is made to expose the trachea and jugular area. A catheter (PE50) is placed in the jugular for administration of compound and maintenance anesthesia. The trachea is incised and a 14-16-gauge modified intravenous catheter is inserted and tied in place as an endotracheal tube. The animal is placed in right lateral recumbency and initially placed on a Harvard ventilator with a tidal volume of 5-10 ml/kg. 100% O₂ is delivered to the animals by the ventilator. ECG electrodes are placed to record a standard Lead II ECG. The surgical site is cleaned with alcohol swab, and a skin incision is made over rib cage over the 4^{th}-5^{th} intercostal space. The underlying muscles are dissected with care to avoid the lateral thoracic vein, to expose the intercostal muscles. The chest cavity is entered through 4^{th}-5^{th} intercostal space, and the incision expanded to allow visualization of the heart. The pericardium is opened to expose the heart. A 6-0 silk suture with a taper needle is passed around the left coronary artery near its origin, which lies in contact with the left margin of the pulmonary cone, at about 1 mm from the insertion of the left auricular appendage. A piece of tubing is placed over the suture to form an occluder. The coronary artery is occluded for 30 minutes by sliding the tube towards the heart until resistance is felt and holding it in place with a vascular clamp. The ECG is monitored for S-T changes indicative of ischemia. After 30 minutes, the occluder is removed, leaving the suture in place. The ECG is monitored for the first 10 minutes of reperfusion. The rat is transferred to the pressure control ventilator for the remainder of the protocol. The rats are ventilated by a small animal ventilator with a peak inspiratory pressure of 10-15 cm H₂O and respiratory rate 60-110 breaths/min. The heart is allowed to reperfuse for 90 minutes.

Surgical procedure for 24 hour study

Rats are anaesthetized with Ketamine/Xylazine IP (95 and 5 mg/kg) and intubated with a 14-16-gauge modified intravenous catheter. Anesthesia level is checked every 15 minutes by toe pinch. Core body temperature is maintained at 37° C. by using a heating blanket. The surgical area is shaved and scrubbed. A ventral midline incision is made to expose the jugular vein. A catheter (PE50) is placed in the jugular for administration of compound and maintenance anesthesia. The animal is placed in right lateral recumbency and initially placed on a ventilator with a tidal volume of 5-10 ml/kg H₂O or a pressure controlled ventilator with a peak inspiratory pressure of 8-15 cm H₂O and respiratory rate 60-110 breaths/min. 100% O₂ is delivered to the animals by the ventilator. ECG electrodes are placed to record a standard Lead II ECG. The surgical site is cleaned with surgical scrub and alcohol. A skin incision is made over rib cage over the 4^{th}-5th intercostal space. The underlying muscles are dissected with care to avoid the lateral thoracic vein, to expose the intercostal muscles. The chest cavity is entered through 4^{th}-5th intercostal space, and the incision expanded to allow visualization of the heart. The pericardium is opened to expose the heart. A 6-0 silk suture with a taper needle is passed around the left coronary artery near its origin, which lies in contact with the left margin of the pulmonary cone, at about 1 mm from the insertion of the left auricular appendage. A piece of tubing is placed over the suture to form an occluder. The coronary artery is occluded for 30 minutes by sliding the tube towards the heart until resistance is felt and holding it in place with a vascular clamp. The ECG is monitored for S-T changes indicative of ischemia. After 30 minutes, the occluder is removed, leaving the suture in place. The ECG is monitored for the first 10 minutes of reperfusion. The incision is closed in three layers. The IV catheter is removed or tunneled under the skin and exteriorized between the shoulder blades to allow for blood withdrawal or further drug therapy. The rat is ventilated until they are able to ventilate on their own. The rats are extubated and recovered on a heating pad. Once awake, they are returned to their cage(s). Animals may receive Buprenorphine (0.01-0.05 mg/kg SQ) for post-operative analgesia. After the designated reperfusion time (24 hours) the animals are anesthetized and the hearts removed under deep anesthesia.

Treatment Protocols

Diet

Animals are fed a custom diet prior to or after coronary ligation. The length of treatment varies with the study. Doses are calculated based on the average consumption of 15 gms of feed per day for a 300 gm rat. Rat weights are monitored during the study. Feed not consumed is weighed to estimate consumption rates.

Gavage

Animals are dosed orally by gavage. Length and frequency of treatment vary withs the study. A standard rat gavage tube (Popper & Sons Inc, New York) is attached to a 3-cc hypodermic syringe. The animal is held by the shoulder in a vertical position. The feeding tube is placed into the mouth then advanced until it reaches the stomach (the approximate insertion length of the tube is measured prior to the feeding). The content of the syringe is slowly delivered, and then the tube is withdrawn.

IV Treatment

A ventral incision is made to expose the jugular area. A catheter (PE50) is placed in the jugular vein for administration of compound. Animals are dosed by bolus injection and/or continuous infusion. The time and duration of treatment varies with the protocol.

Tissue Processing

After reperfusion, each animal receives 200 units of heparin IV under general anesthesia and the heart is removed and placed in cold saline. After removal the coronary artery is ligated with the suture that is already in place. The heart is placed on a perfusion apparatus and Evans Blue dyed is infused delineate the area at risk. The heart is then cut into five 2-mm thick transverse slices from apex to base. The slices are incubated in 1% triphenyltetrazolium chloride (TTC) in 0.9% saline for 20 minutes at 37° C. Tetrazolium reacts with NADH in the presence of dehydrogenase enzymes causing viable tissue to stain a deep red color and that is easily distinguished from the infarcted pale-unstained necrotic tissue. The slices are placed apex side down in the lid of a small petri dish for the staining procedure. The bottom of the dish is placed over the slices to keep them flat. The slices are photographed in order from apex to base, with the base side up. The areas of infarcted tissue, area at risk and the whole left ventricle are determined using a computerized image analysis system. The total area for each region is added together to give a total for the entire heart. Infarct size is expressed both as a percentage of the total ventricle and the area at risk.

Statistical Analysis

Group data is represented as means +/−SEM. Comparisons between treatment groups are made using ANOVA with p<0.05 considered significant. Post hoc comparisons may be made using either Dunnett's test or Tukey's test.

The compounds of the present invention show activity when tested by this method.

### Example 6

### Model of Congestive Heart Failure

Experimental Preparation

225-275 g male sprague-dawley rats were anaesthetized with ketamine/xylazine (95 mg/kg and 5 mg/kg) and intubated with a 14-16-gauge modified intravenous catheter. Core body temperature was maintained at 37° c. by using a heating blanket. The surgical area was clipped and scrubbed, and the animal was placed in right lateral recumbency and initially placed on a ventilator with a peak inspiratory pressure of 10-15 cm H₂O and respiratory rate 60-110 breaths/min. 100% O₂ was delivered to the animals by the ventilator. ECG electrodes were positioned to record a standard lead II ECG. An incision was made over rib cage over the 4th-5th intercostal space. The underlying muscles were dissected with care to avoid the lateral thoracic vein, to expose the intercostal muscles. The chest cavity was entered through 4th-5th intercostal space, and the incision expanded to allow visualization of the heart. The pericardium was opened to expose the heart.

A 6-0 silk suture with a taper needle was passed around the left coronary artery near its origin, about 1 mm from the insertion of the left auricular appendage. The coronary artery was occluded by tying the suture around the artery. The ECG was monitored for S-T changes indicative of ischemia. If the animal developed ventricular fibrillation, gentle cardiac massage was used to convert the animal to a normal rhythm. Sham operated controls were subjected to the same procedure, but the suture was not tied off. The incision was closed in three layers. Infected or moribund animals were eliminated from the study.

Four weeks after surgery, the animals were anesthetized, and a catheter was placed in the right carotid artery and advanced into the left ventricle for hemodynamic measurements. Pressure traces were recorded and analyzed for heart rate, left ventricular systolic and diastolic pressure, left ventricular developed pressure, and dp/dt max and min. After measurements were taken, 2 ml blood was removed and placed in serum and plasma tubes. The heart was removed and placed on a Langendorff apparatus as follows:

Langendorff Procedure

Buffer preparation: Krebs-Henseleit (kh) buffer solution containing NaCl 118 mmol/l, KcL 4.7 mmol/l, MgSO₄ 1.2 mmol/l, KhPO₄ 1.2 mmol/l, glucose 11 mmol/l, NaHCO₃ 25 mmol/l and CaCl₂ 2.5 mmol/l (sigma) was made up fresh each day using nanopure pyrogen-free water.

The animal received 200 units of heparin, the thorax was opened and the heart was rapidly excised and placed in ice-cold K-H buffer solution. After the contractile activity of the heart completely ceased, the heart was trimmed and the ascending aorta freed from the connective tissue. The heart was quickly weighed, then the aorta was cannulated, and the heart mounted on a non-recirculation langendorff perfusion apparatus (radnoti glass technology, inc., Monrovia, Calif.). The heart was perfused in a retrograde fashion via the aorta with kh buffer solution oxygenated with 95% O₂ and 5% CO₂ to maintain ph 7.4 at 37 C. To assess contractile function, a latex balloon was inserted into the left ventricle through the mitral orifice and connected to a pressure transducer by rigid polyethylene tubing. The balloon was inflated with water to a left ventricular end-diastolic pressure (LVEDP) of 1 to 10 mm hg. Flow was initiated at 12 ml/min and adjusted during the first 15 minutes of baseline to obtain a perfusion pressure between 65 and 75 MMHG. Target parameters for baseline were as follows:

Perfusion Pressure 65-75 MMHG

LVEDP 10 MMHG

The heart was allowed to stabilize for 15 minutes. After this time functional measurements were taken, after which a pressure volume curve was generated by adjusting the volume in the balloon in 0.05 ml increments and recording ventricular pressures. The left ventricular systolic pressure (lvsp), left ventricular end diastolic pressure (lvedp), left ventricular developed pressure (lvdp), first derivative of the rise and fall in the left ventricular pressure (dp/dt max, dp/dt min), perfusion pressure and heart rate was automatically recorded using a computerized data acquisition system.

Other Measurements

After removal of the heart, lungs and liver were weighed. The lungs and liver were weighed and dried overnight for determination of wet to dry ratios.

After completing the Langendorff procedure, the heart was placed in cold saline to stop the beating, then cut into five 2-mm thick transverse slices from apex to base. Slice #3 was incubated in 1% triphenyltetrazolium chloride (ttc) in 0.9% saline for 20 minutes at 37° C. Tetrazolium reacts with NADH in the presence of dehydrogenase enzymes causing viable tissue to stain a deep red color and that is easily distinguished from the infarcted pale-unstained necrotic tissue. The slice was placed apex side down in the lid of a small petri dish for the staining procedure. The bottom of the dish was placed over the slice to keep it flat. The slice was then photographed and the areas of infarcted tissue, left and right ventricle weredetermined using a computerized image analysis system. Infarct size was expressed as a percentage of the total ventricle. Total area of the left and right ventricle was measured. The remaining sections were divided into right and left ventricle and frozen for tbars and glutathione assays.

Treatment Protocol

No treatment wasgiven to the sham operated and control groups.

Measurements for CHF Study

In vivo measurements were made of heart rate (hr), left ventricular systolic pressure (lvsp), left ventricular end diastolic pressure (lvedp), dp/dt min and max, right ventricular systolic pressure (rvsp), right ventricular diastolic pressure (rvdp), and right ventricular end diastolic pressure (rvedp), as well as total body weight. Ex vivo measurements were made of hr, lvsp, lvedp, dp/dt min and max, and pressure volume curve. Also measured ex vivo were heart weight, infarct size, gpx, catalase, tbars, glutathione ratio (gsh/gssg), lung and liver wet to dry weight ratios, serum isoprostane and interleukin-6 (IL-6).

In this model, increased heart weight subsequent to permanent ligation of the left coronary artery is used as a measure of cardiac failure; reduction of heart weight after intervention (drug treatment) indicates improvement. In experiments carried out in support of the present invention, intervention with a gamma-tocopherol enriched composition (>65% gamma-tocopherol) resulted in reduction in heart weight with no decrease in left ventricular systolic pressure (LVSP). By way of comparison, captopril (1-[(2S)-3-mercapto-2-methylpropionyl]-L-proline), an ACE-I inhibitor used to treat CHF also reduced heart weight, but this treatment also resulted in reduced LVSP. Clinically, maintenance of LVSP levels is considered a desired outcome in CHF, since reduction in LVSP indicates reduced perfusion pressure.

### Example 7

Example 7 describes a an exemplary energetically competent cells assay

GCL1 Cell Culture

The GCL1 liver cell line was used to screen compounds for prevention of ischemic damage, as described below. This cell line is a hybrid originating from the fusion of rat hepatoma cells (fao) and human fibroblasts (W138). Cassio et al. (1991) J. Cell Biol. 115:1397-1408; Shanks et al. (1994) J. Cell Science 107:813-825. When GCL1 cells are cultured under optimal conditions they attain maximal density in a monolayer and exhibit structural and functional characteristics of a polarized hepatocyte. Ihrke et al. 1993, J. Cell Biol. 123:1761-1775. At greater than 80% confluence, more than 80% of the cells participate in forming one or more phaselucent, spherical structures that are located between cells, termed bile canicular-like spaces. In contrast to the majority of cell lines maintained in culture which are fermentative, GCL1 cells, when at their maximal density and polarity, are highly oxidative (i.e., they can be readily killed by inhibitors of oxidative phosphorylation or electron transport uncouplers). Such cells grown under such conditions are useful for screening compounds and chemical libraries to identify cytoprotective tocopherols that may be useful to treat ischemic injury. These cells are amenable to use in high throughput screening (HTS) technology.

GCL1 growth media is a modified F-12 Casio Nutrient Mixture (Gibco BRL Cat. #94-5147EL). Other reagents used in the culture of GCL1 cells include 0.05% trypsin solution, Phosphate Buffered saline (PBS; Irvine Scientific Cat. #9240) and Fetal Bovine Serum (FBS; Hyclone #SH30070.03).

The GCL1 cell line, obtained from Johns Hopkins University, and passage numbers 3 to 9 were stored frozen. Frozen GCL1 cells were thawed rapidly (within 60-80 seconds) in a 37° C. water bath. As soon as the ice melted, the vial was removed from the water bath and the cell suspension was transferred to 25 ml of growth media in a 75 cm² culture flask (T75; Corning 430641). The cells were evenly distributed in the growth media and were incubated at 37° C. with 7% CO₂ in air atmosphere. The GCL1 cell line grows in monolayers and takes approximately 7 days to reach confluence when seeded at 10⁶ cells per T75 flask. During this time the cells were microscopically examined and the growth media changed every 2 days. The culture media was pre-warmed before use.

Once the cells reached confluence, they were subcultured and used to seed other T75 flasks or opaque 96-well cell culture plates (Costar 3603 or 3916). The growth media was removed from the tissue culture flask(s) and the cells were carefully washed with PBS (approximately 20 ml for a T75 flask). The PBS was removed and replaced with 2 ml of 0.05% Trypsin solution. After 4-5 minutes at room temperature, or until the cells begin to loosen from the vessel surface, the flask was tapped gently to complete the cell detachment. The cell suspension was diluted with 8 ml of fresh growth media. The cells were counted using a hemocytometer and, from a confluent T75 flask, approximately 10⁷ cells were harvested.

To seed T75 flasks, 10⁶ cells are added to the flask and diluted with 25 mL of media. For 96-well plates, 10⁶ cells per plate at 200 μL/well (approximately 10⁴ cells/well) are seeded. To set up 96-well plates, a multichannel Pipettor (12-channel or automatic 8-channel) may be used to distribute the cell suspension. Before seeding into each plate, the cell suspension is mixed to ensure the cells are uniformly distributed. The growth media is replaced with 200 μL/well of fresh media plates every day with the exception of the day after the plates are seeded. The cells can be used for assays 6-8 days after they are seeded

Chemical Ischemia: Antimycin A (AA) Treatment

AA is an inhibitor of the mitochondrial cytochrome bc1 complex and inhibits cellular respiration in a dose-dependent manner. Suzuki et al. (1998) Biochem. Biophys. Res. Commun. 249:542-545.

GCL1 cells were seeded onto 96-well plates at 1×10³/well or 2.5×10³/well as passage 15. The cells were grown in DMEM with 10% FBS and 1.5 g/L sodium bicarbonate for 24 or 48 hours to reach 85% confluence. The cells were washed once with HBSS and then 100 μl of HBSS containing different concentration of AA (Sigma Chemical Co., Cat #A 8674) is added to the cells. AA was dissolved in DMSO to make a stock solution of 50 mM, then diluted with HBSS to 100, 50, 25, 12.5, 6.25, 3.125, and 1.56 μM for dose-response curves. The cells, with or without AA, were incubated at 37° C. in a 5% CO₂ humidified incubator for 1, 3, and 5 hours. The concentration of AA is chosen to induce 75% or 100% cell death as a screening dose to test the effect of tocopherols to protect against AA-induced cell toxicity. The concentration of agents used for screening will be dependent upon the agents. Certain control groups contained 100 μl of HBSS only.

The cell toxicity was evaluated at the end of treatment. The cytotoxicity can measured, for example, by SYTOX (Molecular Probe), Live/Dead Viability /Cytotoxicity Kit (Calcein AM/Ethidium Homodimer-1, Molecular Probe), Alamar blue (Accumed), or LDH assay (Sigma Chemical Co.), according to methods well known in the art or as described herein.

Assessment of Cell Viability

1. LDH Measurement

The hypoxia/reoxygenation or chemical-induced cell toxicity was evaluated by measuring lactate dehydrogenase (LDH) activity released into the bathing medium. 50 μl of HBSS collected from each well is added to a 96 well plate and then mixed with 100 μl reagent from LD-L 20 Kit (Sigma, Cat #228-20). The plate is immediately placed into the SpectraMax 340 and read at 340 nm wavelength at 25° C. for 3 minutes at 30 seconds intervals (kinetic measurement). All of the sample readings subtract the background readings, which is 50 μl HBSS and 100 μl of assay reagent. LDH activity (units/liter) is calculated by kinetic Softmax PRO software, one unit of LDH activity is defined as that amount of enzyme that catalyzed the formation of one micromole of NADH per minute under the condition of the assay procedure. All data is expressed as mean±S.D. from duplicates for 4 culture wells. A paired t-test is performed comparing treated groups (Toxin+Tocopherol) with non-treated groups (Toxin only) after subtracting the control groups (HBSS only) for each data set. Comparisons among groups are statistically evaluated by two-tailed t-test (Graphpad prism Software, version 2.0). Differences are considered significant at P<0.05.

SYTOX Measurement

SYTOX Green nucleic acid stain is a high-affinity nucleic acid stain that easily penetrates cells with a compromised plasma membrane but will not cross the membrane of live cells. The nucleic acids of dead cells fluoresce bright green at 524 nm when excited with the 488 nm spectral source or with any other 450-500 nm source. Hence during a time course with cells incubated with SYTOX, the fluorescent emission at 524 nm is proportional to cell death. In the cell viability assay chemical ischemia is created in cells that are incubated with the SYTOX dye. The compound antimycin is used to simulate ischemia at the same time as stimulating the cells with forskolin. Antimycin inhibits electron transport from FADH₂ between complexes II and III and hence lowers cellular ATP synthesis. The assay is performed on cells grown in 96-well format allowing high throughput screening of compounds.

Materials: (i) SYTOX Green dye, supplied as a 5 mM solution in DMSO (Molecular Probes #S-7020) is stored at −20° C. until use. ii.) Antimycin-A: (Sigma #A-8674) dissolve 5.28 g in 10 ml of DMSO to make a 10 mM stock solution. Aliquot at 100 μl. This 10 mM stock is then diluted to 1 mM with DMSO and aliquoted at 25 μL. Both the 10 mM and the 1 mM are stored at −20° C. until use. (iii) Forskolin (Sigma #F-6886), dissolve 50 mg aliquot in 1.62 ml of DMSO to make a 100 mM stock solution. Aliquot at 100 μl and store at −20° C. until use. iv.) HBSS-SYTOX-FORSKOLIN-ANTIMYCIN (H-S-F-A) solution: H-S-F-A solution is made up fresh prior to each assay in sterile 175 ml or 225 ml Falcon bottles. The working concentrations for the assay are 3 μM SYTOX, 50 μM Forskolin and 30 μM Antimycin.

In each experiment, a 1:500 dilution with HBSS from the 500 μM stock solution was made to generate a 1 μM final working solution of SYTOX Green. The final concentration of DMSO is 0.2%.

Cells were grown in in 96-well black plates (with or without clear bottoms) at a density of 1000 cells per well for 24 hours. The necessary number of 96-well plates containing the specific cell type were obtained and placed in the laminar flow safety cabinet. A sterile microtiter basin was filled with the appropriate volume of pre-warmed 1×HBSS. Using aseptic technique and an 8-channel aspirator, the media was carefully removed from the cells and replaced with 200 μl of 1×HBSS. This is done as quickly as possible to prevent the cells drying out. The plates were then placed in the humidified 37° C. incubators of the Biomek 2000 Side Loader. Four plates were washed at a time so as to minimize the time that the cells are sitting in 1×HBSS prior to addition of the H-S-A-compound test solution.

Candidate compound(s) were mixed with the 1 μM SYTOX working solution and added to the washed cell culture wells. Fluorescence is measured at designated time points (488 nm excitation and 530 nm emission). All of the sample readings subtract the background readings, which is cell-free wells only containing UBSS buffer with SYTOX green dye. The data were compared to non-treated cells (control) and are expressed as mean±S.D. from 4 culture wells. SYTOX Green fluorescence units obtained from each group are corrected for basal fluorescence (cell-free wells with only HBSS and fluorescence reagent). Data are represented as mean±SD. A paired t-test was performed comparing treated groups (Toxin+Agent) with non-treated groups (Toxin only) after subtracting the control groups (HBSS only) for each data set. Comparisons among groups are statistically evaluated by two-tailed t-test (Graphpad Prism Software, version 2.0). Differences are considered significant at P<0.05.

FIG. 7 shows the results of energetically competent cells grown as described above and subjected to oxidative injury. In the experiment shown, a sub-optimal concentration of a flavonoid was used alone or in the presence of varying concentration of Fe3+. It can be seen that the resulting metal chelate provided synergistic protective activity, as compared to either component alone.

We claim:

1. A method for treating and/or ameliorating the symptoms of a tissue ischemic condition in a mammalian subject, comprising administering to the subject an effective amount of a beta-tocopherol enriched tocopherol composition, and by said administering, reducing tissue damage related to said tissue ischemic condition.

2. A method for treating and/or ameliorating the symptoms of a tissue ischemic condition in a mammalian subject, comprising administering to the subject an effective amount of a beta-tocopherol metabolite enriched composition, and by said administering, reducing tissue damage related to said tissue ischemic condition.

3. The method of claim 1 wherein said tissue ischemic condition is selected from the group consisting of cerebral ischemia; intestinal ischemia; spinal cord ischemia; cardiovascular ischemia; myocardial ischemia associated with myocardial infarction; myocardial ischemia associated with CHF, ischemia associated with age-related macular degeneration (AMD); liver ischemia; kidney ischemia; dermal ischemia; vasoconstriction-induced tissue ischemia; penile ischemia as a consequence of priapism; ischemia associated with thromboembolytic disease; ischemia associated with microvascular disease; and ischemia associated with diabetic ulcers, gangrenous conditions, post-trauma syndrome, cardiac arrest resuscitation, peripheral nerve damage or neuropathies.

4. The method of claim 1 wherein said tissue ischemic condition is cerebral ischemia.

5. The method of claim 1 wherein said tissue ischemia is myocardial ischemia associated with myocardial infarction.

6. The method of claim 1 wherein said tissue ischemia is myocardial ischemia associated with CHF.

7. The method of claim 1 wherein said tissue ischemia is ischemia associated with microvascular disease.

8. The method of claim 1 wherein said beta-tocopherol enriched tocopherol composition comprises at least 50% beta-tocopherol.

9. The method of claim 1 wherein said beta-tocopherol enriched tocopherol composition comprises at least 75% beta-tocopherol.

10. The method of claim 1 wherein said beta-tocopherol enriched tocopherol composition comprises at least 90% beta-tocopherol.

11. The method of claim 2 wherein said beta-tocopherol metabolite enriched composition comprises at least 50% beta-tocopherol metabolite.

12. The method of claim 2 wherein said beta-tocopherol metabolite enriched composition comprises at least 75% beta-tocopherol metabolite.

13. The method of claim 2 wherein said beta-tocopherol metabolite enriched composition comprises at least 90% beta-tocopherol metabolite.

14. The method of claim 1 wherein said composition is a nutritional composition.

15. The method of claim 1 wherein said composition is a pharmaceutical composition.

16. The method of claim 1 wherein said composition is administered orally.

17. The method of claim 1 wherein said composition is administered parenterally.

18. The method of claim 1 wherein said composition comprises a beta-tocopherol in a range of about 1 to about 1000 mg per kg body weight of said mammalian subject.

19. The method of claim 1 wherein said composition comprises a beta-tocopherol in a range of about 1 to about 50 mg per kg body weight of said mammalian subject.

20. The method of claim 1 wherein said composition comprises a beta-tocopherol in a range of about 10 to about 100 mg per kg body weight of said mammalian subject.

21. A method for treating and/or ameliorating the symptoms of a tissue ischemic condition in a mammalian subject, comprising administering to the subject an effective amount of a delta-tocopherol enriched tocopherol composition, and by said administering, reducing tissue damage related to said tissue ischemic condition.

22. A method for treating and/or ameliorating the symptoms of a tissue ischemic condition in a mammalian subject, comprising administering to the subject an effective amount of a delta-tocopherol metabolite enriched composition, and by said administering, reducing tissue damage related to said tissue ischemic condition.

23. The method of claim 21 wherein said tissue ischemic condition is selected from the group consisting of cerebral ischemia; intestinal ischemia; spinal cord ischemia; cardiovascular ischemia; myocardial ischemia associated with myocardial infarction; myocardial ischemia associated with CHF, ischemia associated with age-related macular degeneration (AMD); liver ischemia; kidney ischemia; dermal ischemia; vasoconstriction-induced tissue ischemia; penile ischemia as a consequence of priapism; ischemia associated with thromboembolytic disease; ischemia associated with microvascular disease; and ischemia associated with diabetic ulcers, gangrenous conditions, post-trauma syndrome, cardiac arrest resuscitation, peripheral nerve damage or neuropathies.

24. The method of claim 21 wherein said tissue ischemia is cerebral ischemia.

25. The method of claim 21 wherein said tissue ischemia is myocardial ischemia associated with myocardial infarction.

26. The method of claim 21 wherein said tissue ischemia is myocardial ischemia associated with CHF.

27. The method of claim 21 wherein said tissue ischemia is ischemia associated with microvascular disease.

28. The method of claim 21 wherein said delta-tocopherol enriched tocopherol composition comprises at least 50% delta-tocopherol.

29. The method of claim 21 wherein said delta-tocopherol enriched tocopherol composition comprises at least 75% delta-tocopherol.

30. The method of claim 21 wherein said delta-tocopherol enriched tocopherol composition comprises at least 90% delta-tocopherol.

31. The method of claim 22 wherein said delta-tocopherol metabolite enriched composition comprises at least 50% delta-tocopherol metabolite.

32. The method of claim 22 wherein said delta-tocopherol metabolite enriched composition comprises at least 75% delta-tocopherol metabolite.

33. The method of claim 22 wherein said delta-tocopherol metabolite enriched composition comprises at least 90% delta-tocopherol metabolite.

34. The method of claim 21 wherein said composition is a nutritional composition.

35. The method of claim 21 wherein said composition is a pharmaceutical composition.

36. The method of claim 21 wherein said composition is administered orally.

37. The method of claim 21 wherein said composition is administered parenterally.

38. The method of claim 21 wherein said composition comprises a delta-tocopherol in a range of about 1 to about 1000 mg per kg body weight of said mammalian subject.

39. The method of claim 21 wherein said composition comprises a delta-tocopherol in a range of about 1 to about 50 mg per kg body weight of said mammalian subject.

40. The method of claim 21 wherein said composition comprises a delta-tocopherol in a range of about 10 to about 100 mg per kg body weight of said mammalian subject.

41. A method for treating and/or ameliorating the symptoms of a non-cardiovascular tissue ischemic condition in a mammalian subject, comprising administering to the subject an effective amount of a gamma-tocopherol enriched tocopherol composition, and by said administering, reducing tissue damage related to said non-cardiovascular tissue ischemic condition.

42. A method for treating and/or ameliorating the symptoms of a non-cardiovascular tissue ischemic condition in a mammalian subject, comprising administering to the subject an effective amount of a gamma-tocopherol metabolite enriched composition, and by said administering, reducing tissue damage related to said non-cardiovascular tissue ischemic condition.

43. The method of claim 41 wherein said non-cardiovascular tissue ischemic condition is selected from the group consisting of intestinal ischemia; spinal cord ischemia; ischemia associated with age-related macular degeneration (AMD); liver ischemia; kidney ischemia; dermal ischemia; vasoconstriction-induced tissue ischemia; penile ischemia as a consequence of priapism; ischemia associated with thromboembolytic disease; ischemia associated with microvascular disease; and ischemia associated with diabetic ulcers, gangrenous conditions, post-trauma syndrome, peripheral nerve damage or neuropathies.

44. The method of claim 41 wherein said gamma-tocopherol enriched tocopherol composition comprises at least 60% gamma-tocopherol.

45. The method of claim 41 wherein said gamma-tocopherol enriched tocopherol composition comprises at least 65% gamma-tocopherol.

46. The method of claim 41 wherein said gamma-tocopherol enriched tocopherol composition comprises at least 70% gamma-tocopherol.

47. The method of claim 41 wherein said gamma-tocopherol enriched tocopherol composition comprises at least 75% gamma-tocopherol.

48. The method of claim 41 wherein said gamma-tocopherol enriched tocopherol composition comprises at least 80% gamma-tocopherol.

49. The method of claim 41 wherein said gamma-tocopherol enriched tocopherol composition comprises at least 85% gamma-tocopherol.

50. The method of claim 41 wherein said gamma-tocopherol enriched tocopherol composition comprises at least 90% gamma-tocopherol.

51. The method of claim 41 wherein said gamma-tocopherol enriched tocopherol composition comprises at least 95% gamma-tocopherol.

52. The method of claim 41 wherein said gamma-tocopherol enriched tocopherol composition comprises at least 98% gamma-tocopherol.

53. The method of claim 42 wherein said gamma-tocopherol metabolite enriched composition comprises at least 80% gamma-tocopherol metabolite.

54. The method of claim 42 wherein said gamma-tocopherol metabolite enriched composition comprises at least 85% gamma-tocopherol metabolite.

55. The method of claim 42 wherein said gamma-tocopherol metabolite enriched composition comprises at least 90% gamma-tocopherol metabolite.

56. The method of claim 42 wherein said gamma-tocopherol metabolite enriched composition comprises at least 95% gamma-tocopherol metabolite.

57. The method of claim 42 wherein said gamma-tocopherol metabolite enriched composition comprises at least 98% gamma-tocopherol metabolite.

58. The method of claim 41 wherein said composition is a nutritional composition.

59. The method of claim 41 wherein said composition is a pharmaceutical composition.

60. The method of claim 41 wherein said composition is administered orally.

61. The method of claim 41 wherein said composition is administered parenterally.

62. The method of claim 41 wherein said composition comprises gamma-tocopherol in a range of about 1 to about 1000 mg per kg body weight of said mammalian subject.

63. The method of claim 41 wherein said composition comprises gamma-tocopherol in a range of about 1 to about 50 mg per kg body weight of said mammalian subject.

64. The method of claim 41 wherein said composition comprises gamma-tocopherol in a range of about 10 to about 100 mg per kg body weight of said mammalian subject.

65. A method for treating and/or ameliorating the symptoms of a tissue ischemic condition in a mammalian subject, comprising administering to the subject an effective amount of a composition comprising a flavonoid and/or a flavonoid derivative, and by said administering, reducing tissue damage related to said tissue ischemic condition, wherein said flavonoid specifically excludes diosmin and hesperidin.

66. The method of claim 65 wherein said tissue ischemic condition is selected from the group consisting of intestinal ischemia; spinal cord ischemia; ischemia associated with age-related macular degeneration (AMD); liver ischemia; kidney ischemia; dermal ischemia; vasoconstriction-induced tissue ischemia; penile ischemia as a consequence of priapism; ischemia associated with thromboembolytic disease; ischemia associated with microvascular disease; and ischemia associated with diabetic ulcers, gangrenous conditions, post-trauma syndrome, peripheral nerve damage or neuropathies.

67. The method of claim 65 wherein said tissue ischemic conditions is cerebral ischemia.

68. The method of claim 65 wherein said flavonoid is chelated to a metal.

69. The method of claim 68 wherein said metal is Fe.

70. The method of claim 65 wherein said flavonoid is selected from the group consisting of chrysin, daidzein, hesperetin, luteolin, quercetin, bromoquercetin, rutin, and biochanin.

71. The method of claim 65 wherein said composition comprises two flavonoids.

72. The method of claim 65 wherein said flavonoid is quercetin.

73. The method of claim 65 wherein said flavonoid is hesperetin.

74. The method of claim 65 wherein said flavonoid is bromoquercetin.

75. The method of claim 65 wherein said composition comprises quercetin and hesperetin.

76. The method of claim 65 wherein said composition is a nutritional composition.

77. The method of claim 65 wherein said composition is a pharmaceutical composition.

78. The method of claim 65 wherein said composition is administered orally.

79. The method of claim 65 wherein said composition is administered parenterally.

80. The method of claim 65 wherein said composition comprises a flavonoid in a range of about 1 to about 1000 mg per kg body weight of said mammalian subject.

81. The method of claim 65 wherein said composition comprises a flavonoid in a range of about 1 to about 25 mg per kg body weight of said mammalian subject.

82. A method for treating and/or ameliorating the symptoms of a tissue ischemic condition in a mammalian subject, comprising administering to the subject an effective amount of a composition comprising a non-alpha tocopherol and a flavonoid, wherein said flavonoid is selected from the group consisting of chrysin, daidzein, diosmin, hesperetin, hesperidin, luteolin, quercetin, bromoquercetin, rutin and biochanin, and by said administering, reducing tissue damage related to said tissue ischemic condition.

83. The method of claim 82 wherein said tissue ischemic condition is cerebral ischemia.

84. The method of claim 82 wherein said tissue ischemic condition is cardiovascular ischemia.

85. The method of claim 82 wherein said non-alpha tocopherol is selected from the group consisting of gamma-tocopherol, beta-tocopherol, delta-tocopherol, a gamma-tocopherol metabolite, a beta-tocopherol metabolite, and a delta-tocopherol metabolite.

86. The method of claim 82 wherein said non-alpha tocopherol is gamma-tocopherol.

87. The method of claim 82 wherein said non-alpha-tocopherol is a gamma-tocopherol metabolite.

88. The method of claim 82 wherein said composition comprises gamma-tocopherol and two flavonoids.

89. The method of claim 82 wherein said composition comprises gamma-tocopherol, quercetin and hesperetin.

90. The method of claim 82 wherein said flavonoid is metal chelated.

91. The method of claim 82 wherein said composition comprises gamma-tocopherol in the range of about 1 to about 1000 mg/kg body weight of mammalian subject, hesperetin in the range of about 1 to about 1000 mg/kg body weight of mammalian subject and quercetin in a range of about 1 to about 1000 mg/kg body weight of mammalian subject.

92. The method of claim 82 wherein said composition comprises gamma-tocopherol in the range of about 1 to about 50 mg/kg body weight of mammalian subject, hesperetin in the range of about 1 to about 25 mg/kg body weight of mammalian subject and quercetin in a range of about 1 to about 25 mg/kg body weight of mammalian subject.

93. The method of claim 82 wherein said composition comprises gamma-tocopherol in the range of about 10 to about 100 mg/kg body weight of mammalian subject, hesperetin in the range of about 10 to about 100 mg/kg body weight of mammalian subject and quercetin in a range of about 10 to about 100 mg/kg body weight of mammalian subject.

94. The method of claim 82 wherein said composition is a nutritional composition.

95. The method of claim 82 wherein said composition is a pharmaceutical composition.

96. The method of claim 82 wherein said composition is administered orally.

97. The method of claim 82 wherein said composition is administered parenterally.
